(19) United States
(12) Patent Application Publication
(10) Pub. No.: US 2024/0197839 A1
(43) Pub. Date: Jun. 20, 2024

(54) Recombinant Human Acid Alpha-Glucosidase and Uses Thereof

(71) Applicant: Amicus Therapeutics, Inc., Philadelphia, PA (US)

(72) Inventors: Hung V. Do, New Hope, PA (US); Russell Gotschall, Doylestown, PA (US); Hing Char, East Brunswick, NJ (US); Jay Barth, Teaneck, NJ (US)

(73) Assignee: Amicus Therapeutics, Inc., Philadelphia, PA (US)

(21) Appl. No.: 18/276,954

(22) PCT Filed: Feb. 11, 2022
(86) PCT No.: PCT/US2022/016124

§ 371 Date: Aug. 11, 2023

Related U.S. Application Data
(60) Provisional application No. 63/162,683, filed on Mar. 18, 2021.
(60) Provisional application No. 63/148,596, filed on Feb. 11, 2021.

(51) Int. Cl. A61K 38/47; A61P 3/00; A61P 11/00; A61P 21/00

(52) U.S. Cl. CPC A61K 38/47; A61P 3/00; A61P 11/00; A61P 21/00; C12Y 302/0102

(57) ABSTRACT

Provided herein are methods of treating Pompe disease comprising administering a population of recombinant human acid a-glucosidase molecules or a pharmaceutical composition or formulation thereof, and a pharmacological chaperone.

### CROSS-REFERENCE TO RELATED APPLICATIONS

This application claims the benefit of U.S. Provisional Patent Application No. 63/162,683, filed on Mar. 18, 2021, and U.S. Provisional Patent Application No. 63/148,596, filed on Feb. 11, 2021, the disclosure of each of which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AMCS_013_02WO_SeqList_ST25.txt, date recorded: Feb. 11, 2022, file size ˜45,560 bytes).

### TECHNICAL FIELD

The disclosure relates to a recombinant human α-glucosidase (rhGAA) and treatments for Pompe disease.

### BACKGROUND

Pompe disease is an inherited lysosomal storage disease that results from a deficiency in acid α-glucosidase (GAA) activity. A person having Pompe disease lacks or has reduced levels of acid α-glucosidase (GAA), the enzyme which breaks down glycogen to glucose, a main energy source for muscles. This enzyme deficiency causes excess glycogen accumulation in the lysosomes, which are intra-cellular organelles containing enzymes that ordinarily break down glycogen and other cellular debris or waste products. Glycogen accumulation in certain tissues of a subject having Pompe disease, especially muscles, impairs the ability of cells to function normally. In Pompe disease, glycogen is not properly metabolized and progressively accumulates in the lysosomes, especially in skeletal muscle cells and, in the infant onset form of the disease, in cardiac muscle cells. The accumulation of glycogen damages the muscle and nerve cells as well as those in other affected tissues.

Traditionally, depending on the age of onset, Pompe disease is clinically recognized as either an early infantile form or as a late onset form. The age of onset tends to parallel the severity of the genetic mutation causing Pompe disease. The most severe genetic mutations cause complete loss of GAA activity and manifest as early onset disease during infancy. Genetic mutations that diminish GAA activity but do not eliminate it are associated with forms of Pompe disease having delayed onset and progression. Infantile onset Pompe disease manifests shortly after birth and is characterized by muscular weakness, respiratory insufficiency and cardiac failure. Untreated, it is usually fatal within two years. Juvenile and adult onset Pompe disease manifest later in life and usually progress more slowly than infantile onset disease. This form of the disease, while it generally does not affect the heart, may also result in death, due to weakening of skeletal muscles and those involved in respiration.

Current non-palliative treatment of Pompe disease involves enzyme replacement therapy (ERT) using recombinant alglucosidase alfa products sold under the trademarks LUMIZYME® and MYOZYME®. This conventional enzyme replacement therapy seeks to treat Pompe disease by replacing the missing GAA in lysosomes by administering rhGAA thus restoring the ability of cells to break down lysosomal glycogen. LUMIZYME® and MYOZYME® are conventional forms of rhGAA produced or marketed as biologics by Genzyme and approved by the U.S. Food and Drug Administration, and are described by reference to the Physician's Desk Reference (2014) (which is hereby incorporated by reference). Alglucosidase alfa is identified as chemical name [199-arginine, 223-histidine]prepro-α-glucosidase (human); molecular formula, C₄₇₅sH₇₂₆₂N₁₂₇₄O₁₃₆₉S₃₅; CAS number 420794-05-0. These products are administered to subjects with Pompe disease, also known as glycogen storage disease type II (GSD-II) or acid maltase deficiency disease.

However, the current ERT, at best, offers limited improvement in measures of muscle function, strength and respiratory function for a finite duration followed by slow decline in these parameters (Toscano and Schoser 2013; Wyatt et al 2012).

In 2012, a systematic review of all studies performed in subjects with late-onset Pompe disease (LOPD) was performed by Toscano and Schoser 2013. The review included data on 368 subjects with LOPD from published studies, including 27 juvenile subjects (age range: 2 to 17 years old) and 251 adult subjects who received alglucosidase alfa for at least 2 preceding years. Results indicated that >30% of subjects did not show an initial improvement during treatment with alglucosidase alfa and continued to experience deterioration of muscular and respiratory functions despite treatment. In the group of subjects who initially responded to alglucosidase alfa treatment, several additional longer-term studies showed that improvements usually lasted for only approximately 2 years. Thereafter, subjects generally plateaued before beginning to progressively decline.

In 2012, the United Kingdom Health Technology Assessment program, as part of the National Institute for Health Research (Wyatt et al 2012), issued recommendations drawn from review of longitudinal data for 81 patients with Pompe disease (including infantile- and late-onset forms (children and adults)) who received the current approved ERT standard of care, alglucosidase alfa. Key markers of Pompe disease progression (forced vital capacity, ventilation dependency, mobility, 6-Minute Walk Test, muscle strength, and body mass index) were assessed and modeled with time of treatment on alglucosidase alfa therapy. Results of this assessment indicated that improvements in FVC, 6-minute walk distance, and muscle strength by patients with LOPD occurred for the first 2 years after commencing ERT with alglucosidase alfa, and decline occurred with continuing treatment beyond this timeframe. Additionally, a 3-year study in 38 subjects with LOPD receiving alglucosidase alfa showed that the subjects demonstrated an improvement in motor function in the first year of treatment, which remained generally stable in the second year and began to decline in the third year (Regnery et al 2012).

Furthermore, a report providing a 10-year follow-up on a Phase 3 LUMIZYME® (Genzyme Corporation) study showed that after experiencing some improvement in motor and pulmonary function during the first couple of years of treatment, subjects began to slowly decline with ongoing treatment (van der Ploeg et al 2017). In the study, from years 3 to 6 on therapy, there was an average decline of approximately 10% in percent predicted baseline 6 minute walk distance, with approximately 80% of subjects experiencing a decline.

The most serious tolerability issue with alglucosidase alfa is the occurrence of infusion-associated reactions (IARs), which, in some instances can include life-threatening anaphylaxis or other severe allergic responses (MYOZYME® Summary of Product Characteristics, December 2018). Management of these events include dose reduction, reduced infusion rates and prolonged infusion times, and dose interruption or discontinuation. Premedication with antihistamines and steroids (prior to infusion) is also regularly used to prevent and reduce the occurrence and severity of IARs and hypersensitivities related to alglucosidase alfa infusion. Despite these measures, patients with Pompe disease may still experience IARs, and some cannot tolerate regular infusions of the currently approved ERT.

In 2017, a systematic review of the literature was undertaken by the European Pompe Consortium, a network of experts from 11 European countries in the field of Pompe disease (van der Ploeg et al 2017). Based on the data obtained from one clinical study and 43 observational studies, covering a total of 586 individual adult subjects, evidence of an effect of ERT at group level was assessed by the consortium. The current European Pompe Consortium consensus is to discontinue ERT therapy upon the occurrence of severe IARs or the progressive clinical worsening of disease symptoms, as well as occurrence of high-neutralizing antibody (Ab) titers, which effectively inactivate the existing ERT treatment. The European Pompe Consortium consensus recommendation also included consideration for re-initiation of ERT treatment if disease progression and clinical worsening recur after ERT has been stopped.

Thus, there remains a need to identify improved rhGAA therapies that are effective to treat Pompe disease with reduced adverse events.

The cellular uptake of a rhGAA molecule is facilitated by the specialized carbohydrate, mannose-6-phosphate (M6P), which binds to the cation-independent mannose-6-phosphate receptor (CIMPR) present on target cells such as muscle cells. Upon binding, rhGAA molecule is taken up by target cells and subsequently transported into the lysosomes within the cells. Most of the conventional rhGAA products, however, lack a high total content of mono-M6P- and bis-M6P-bearing N-glycans (i.e., N-glycans bearing one M6P residue or N-glycans bearing two M6P residues, respectively), which limits their cellular uptake via CIMPR and lysosomal delivery, thus making conventional enzyme replacement therapy insufficiently effective. For example, while conventional rhGAA products at 20 mg/kg or higher doses do ameliorate some aspects of Pompe disease, they are not able to adequately, among other things, (i) treat the underlying cellular dysfunction, (ii) restore muscle structure, or (iii) reduce accumulated glycogen in many target tissues, such as skeletal muscles, to reverse disease progression. Further, higher doses may impose additional burdens on the subject as well as medical professionals treating the subject, such as lengthening the infusion time needed to administer rhGAA intravenously.

The glycosylation of GAA or rhGAA can be enzymatically modified in vitro by the phosphotransferase and uncovering enzymes described by Canfield, et al., U.S. Pat. No. 6,534,300, to generate M6P groups. However, enzymatic glycosylation cannot be adequately controlled and can produce rhGAA having undesirable immunological and pharmacological properties. Enzymatically modified rhGAA may contain only high-mannose oligosaccharide which all could be potentially enzymatically phosphorylated in vitro with a phosphotransferase or uncovering enzyme. The glycosylation patterns produced by in vitro enzymatic treatment of GAA are problematic because the additional terminal mannose residues, particularly non-phosphorylated terminal mannose residues, negatively affect the pharmacokinetics of the modified rhGAA. When such an enzymatically modified product is administered in vivo, these mannose groups increase non-productive clearance of the GAA, increase the uptake of the enzymatically-modified GAA by immune cells, and reduce rhGAA therapeutic efficacy due to less of the GAA reaching targeted tissues, such as skeletal muscle myocytes. For example, terminal non-phosphorylated mannose residues are known ligands for mannose receptors in the liver and spleen which leads to rapid clearance of the enzymatically-modified rhGAA and reduced targeting of rhGAA to target tissue. Moreover, the glycosylation pattern of enzymatically-modified GAA having high mannose N-glycans with terminal non-phosphorylated mannose residues resembles that on glycoproteins produced in yeasts and molds, and increases the risk of triggering immune or allergic responses, such as life-threatening severe allergic (anaphylactic) or hypersensitivity reactions, to the enzymatically modified rhGAA.

Compared with conventional recombinant rhGAA products and in vitro-phosphorylated rhGAA, the rhGAA used in the two-component therapy according to this disclosure has an optimized N-glycan profile for enhanced biodistribution and lysosomal uptake, thereby minimizing non-productive clearance of rhGAA once administered. The present disclosure provides stable or declining Pompe patients an effective therapy that reverses disease progression at the cellular level—including clearing lysosomal glycogen more efficiently than the current standard of care. Patients treated with the two-component therapy of the present disclosure comprising rhGAA and a pharmaceutical chaperone (e.g., miglustat) exhibit significant health improvements, including improvements in muscle strength, motor function, and/or pulmonary function, and/or including a reversal in disease progression, as demonstrated in various efficacy results (e.g., Examples 8 and 9) from the clinical studies.

### SUMMARY

Provided herein is a method of treating a disease or disorder such as Pompe disease in a subject, comprising administering a population of recombinant human acid α-glucosidase (rhGAA) molecules and a pharmacological chaperone (e.g., miglustat).

The rhGAA molecules described herein may be expressed in Chinese hamster ovary (CHO) cells and comprise seven potential N-glycosylation sites. In some embodiments, the N-glycosylation profile of a population of rhGAA molecules as described herein is determined using liquid chromatography-tandem mass spectrometry (LC-MS/MS). In some embodiments, the rhGAA molecules on average comprise 3-4 mol mannose-6-phosphate (M6P) residues per mol of rhGAA. In some embodiments, the rhGAA molecules on average comprise about at least 0.5 mol bis-phosphorylated N-glycan groups (bis-M6P) per mol of rhGAA at the first potential N-glycosylation site. In some embodiments, the rhGAA comprises an amino acid sequence at least 95% identical to SEQ ID NO: 4 or SEQ ID NO: 6. In some embodiments, the rhGAA comprises the amino acid sequence identical of SEQ ID NO: 4 or SEQ ID NO: 6. In some embodiments, at least 30% of molecules of the rhGAA molecules comprise one or more N-glycan units bearing one or two M6P residues. In some embodiments, the rhGAA molecules comprise on average from about 0.5 mol to about 7.0 mol of N-glycan units bearing one or two M6P residues per mol of rhGAA. In some embodiments, the rhGAA molecules comprise on average from 2.0 to 8.0 mol of sialic acid per mol of rhGAA. In some embodiments, the rhGAA molecules comprise on average at least 2.5 moles of M6P residues per mol of rhGAA and at least 4 mol of sialic acid residues per mol of rhGAA. In some embodiments, the rhGAA molecules comprising an average of 3-4 mol M6P residues per mol of rhGAA and an average of about at least 0.5 mol bis-M6P per mol rhGAA at the first potential N-glycosylation site further comprise an average of about 0.4 to about 0.6 mol mono-phosphorylated N-glycans (mono-M6P) per mol rhGAA at the second potential N-glycosylation site, about 0.4 to about 0.6 mol bis-M6P per mol rhGAA at the fourth potential N-glycosylation site, and about 0.3 to about 0.4 mol mono-M6P per mol rhGAA at the fourth potential N-glycosylation site. In some embodiments, the rhGAA molecules further comprise on average about 4 mol to about 7.3 mol of sialic acid residues per mol of rhGAA, including about 0.9 to about 1.2 mol sialic acid per mol rhGAA at the third potential N-glycosylation site, about 0.8 to about 0.9 mol sialic acid per mol rhGAA at the fifth potential N-glycosylation site, and about 1.5 to about 4.2 mol sialic acid per mol rhGAA at the sixth potential N-glycosylation site. In some embodiments, the population of rhGAA molecules is formulated in a pharmaceutical composition. In some embodiments, the pharmaceutical composition comprising a population of rhGAA molecules further comprises at least one buffer selected from the group consisting of a citrate, a phosphate, and a combination thereof, and at least one excipient selected from the group consisting of mannitol, polysorbate 80, and a combination thereof. In some embodiments, the pH of the pharmaceutical composition is about 5.0 to about 7.0, about 5.0 to about 6.0, or about 6.0. In some embodiments, the pharmaceutical composition further comprises water, an acidifying agent, an alkalizing agent, or a combination thereof. In some embodiments, the pharmaceutical composition has a pH of 6.0 and comprises about 5-50 mg/mL of the population of rhGAA molecules, about 10-100 mM of a sodium citrate buffer, about 10-50 mg/mL mannitol, about 0.1-1 mg/mL polysorbate 80, and water, and optionally comprises an acidifying agent and/or alkalizing agent. In some embodiments, the pharmaceutical composition has a pH of 6.0 and comprises about 15 mg/mL of the population of rhGAA molecules, about 25 mM of a sodium citrate buffer, about 20 mg/mL mannitol, about 0.5 mg/mL polysorbate 80, and water, and optionally comprises an acidifying agent and/or alkalizing agent.

In some embodiments, the population of rhGAA molecules is administered at a dose of about 1 mg/kg to about 100 mg/kg or about 5 mg/kg to about 20 mg/kg. In some embodiments, the population of rhGAA molecules is administered at a dose of about 20 mg/kg. In some embodiments, the population of rhGAA molecules is administered bimonthly, monthly, bi-weekly, weekly, twice weekly, or daily, for example, bi-weekly. In some embodiments, the population of rhGAA molecules is administered intravenously.

In some embodiments, the population of rhGAA molecules is administered concurrently or sequentially with a pharmacological chaperone such as miglustat (also referred to as AT2221) or a pharmaceutically acceptable salt thereof. In some embodiments, the miglustat or pharmaceutically acceptable salt thereof is administered orally, for example at a dose of about 50 mg to about 200 mg or from about 200 mg to about 600 mg, and optionally about 130 mg, about 195 mg, or about 260 mg. In some embodiments, the population of rhGAA molecules is administered intravenously at a dose of about 5 mg/kg to about 20 mg/kg and the miglustat or pharmaceutically acceptable salt thereof is administered orally at a dose of about 233 mg to about 500 mg. In some embodiments, the population of rhGAA molecules is administered intravenously at a dose of about 5 mg/kg to about 20 mg/kg and the miglustat or pharmaceutically acceptable salt thereof is administered orally at a dose of about 50 mg to about 200 mg. In one embodiment, the population of rhGAA molecules is administered intravenously at a dose of about 20 mg/kg and the miglustat or pharmaceutically acceptable salt thereof is administered orally at a dose of about 260 mg. In one embodiment, the population of rhGAA molecules is administered intravenously at a dose of about 20 mg/kg and the miglustat or pharmaceutically acceptable salt thereof is administered orally at a dose of about 195 mg. In some embodiments, the miglustat or pharmaceutically acceptable salt thereof is administered prior to (for example, about one hour prior to) administration of the population of rhGAA molecules. In at least one embodiment, the subject fasts for at least two hours before and at least two hours after the administration of miglustat or a pharmaceutically acceptable salt thereof.

Embodiments of the disclosure demonstrate the efficacy of the two-component therapy described herein to treat and reverse disease progression in a subject with Pompe disease. In some embodiments, the subject is an ERT-experienced patient. In some embodiments, the subject is an ERT-naive patient.

In some embodiments, the two-component therapy according to this disclosure improves one or more disease symptoms in a subject with Pompe disease compared to (1) baseline, or (2) a control treatment comprising administering alglucosidase alfa and a placebo for the pharmacological chaperone. In such control treatment, a placebo was administered in place of the pharmacological chaperone.

In some embodiments, the two-component therapy according to this disclosure improves the subject's motor function, as measured by a 6-minute walk test (6MWT). In some embodiments, compared to baseline, the subject's 6-minute walk distance (6MWD) is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks of treatment. In some embodiments, the subject's 6MWD is increased by at least 20 meters or at least 5% after 52 weeks of treatment. In some embodiments, compared to the control treatment, the subject's 6MWD is improved by at least 5, 6, 7, 8, 9, 10, 12, 14, 16, 18, 20, 30, 40, or 50 meters after 12, 26, 38, or 52 weeks of treatment. In some embodiments, compared to the control treatment, the subject's 6MWD is improved by at least 13 meters after 52 weeks of treatment. In some embodiments, the subject has a baseline 6MWD less than 300 meters. In some embodiments, the subject has a baseline 6MWD greater than or equal to 300 meters.

In some embodiments, the two-component therapy according to this disclosure stabilizes the subject's pulmonary function, as measured by a forced vital capacity (FVC) test. In some embodiments, after 12, 26, 38, or 52 weeks of treatment, the subject's percent-predicted FVC is either increased compared to baseline, or decreased by less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% compared to baseline. In some embodiments, after 52 weeks of treatment, the subject's percent-predicted FVC is decreased by less than 1% compared to baseline. In some embodiments, compared to the control treatment, the subject's percent-predicted FVC is significantly improved after treatment. In some embodiments, compared to the control treatment, the subject's percent-predicted FVC is significantly improved by at least 0.5%, 1%, 2%, 3%, 4%, 5%, or 6% after 12, 26, 38, or 52 weeks of treatment. In some embodiments, compared to the control treatment, the subject's percent-predicted FVC is significantly improved by at least 3% after 52 weeks of treatment. In some embodiments, the subject has a baseline FVC less than 55%. In some embodiments, the subject has a baseline FVC greater than or equal to 55%.

In some embodiments, the two-component therapy according to this disclosure improves the subject's motor function, as measured by a gait, stair, gower, chair (GSGC) test. In some embodiments, compared to baseline, the subject's GSGC score is improved as indicated by a decrease of at least 0.1, 0.3, 0.5, 0.7, 1.0, 1.5, or 2.5 points after 12, 26, 38 or 52 weeks of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks of treatment. In some embodiments, compared to the control treatment, the subject's GSGC score is significantly improved as indicated by a decrease of at least 1.0 point after 52 weeks of treatment.

In some embodiments, the two-component therapy according to this disclosure reduces the level of at least one marker of muscle damage after treatment. In some embodiments, the at least one marker of muscle damage comprises creatine kinase (CK). In some embodiments, compared to baseline, the subject's CK level is reduced by at least 10%, 15%, 20%, 25%, 30%, 40%, or 50% after 12, 26, 38, or 52 weeks of treatment. In some embodiments, compared to baseline, the subject's CK level is reduced by at least 20% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks of treatment. In some embodiments, compared to the control treatment, the subject's CK level is significantly reduced by at least 30% after 52 weeks of treatment.

In some embodiments, the two-component therapy according to this disclosure reduces the level of at least one marker of glycogen accumulation after treatment. In some embodiments, the at least one marker of glycogen accumulation comprises urine hexose tetrasaccharide (Hex4). In some embodiments, compared to baseline, the subject's urinary Hex4 level is reduced by at least 10%, 15%, 20%, 25%, 30%, 40%, 50%, or 60% after 12, 26, 38, or 52 weeks of treatment. In some embodiments, compared to baseline, the subject's urinary Hex4 level is reduced by at least 30% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks of treatment. In some embodiments, compared to the control treatment, the subject's urinary Hex4 level is significantly reduced by at least 40% after 52 weeks of treatment.

In some embodiments, the two-component therapy according to this disclosure improves one or more disease symptoms in an ERT-experienced patient subject with Pompe disease compared to (1) baseline, or (2) a control treatment comprising administering alglucosidase alfa and a placebo for the pharmacological chaperone.

In some embodiments, the two-component therapy for an ERT-experienced subject with Pompe disease improves the subject's motor function, as measured by a 6MWT. In some embodiments, compared to baseline, the subject's 6MWD is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks of treatment. In some embodiments, the subject's 6MWD is increased by at least 15 meters or at least 5% after 52 weeks of treatment. In some embodiments, compared to the control treatment, the subject's 6MWD is significantly improved after treatment. In some embodiments, compared to the control treatment, the subject's 6MWD is significantly improved by at least 10, 12, 14, 15, 16, 18, 20, 30, 40, or 50 meters after 12, 26, 38, or 52 weeks of treatment. In some embodiments, compared to the control treatment, the subject's 6MWD is significantly improved by at least 15 meters after 52 weeks of treatment. In some embodiments, the subject has a baseline 6MWD less than 300 meters. In some embodiments, the subject has a baseline 6MWD greater than or equal to 300 meters.

In some embodiments, the two-component therapy for an ERT-experienced subject with Pompe disease improves the subject's pulmonary function, as measured by an FVC test. In some embodiments, after 12, 26, 38, or 52 weeks of treatment, the subject's percent-predicted FVC is increased by at least 0.1%, 0.2%, 0.3%, 0.4%, 0.5%, 1%, 2%, 3%, 4%, or 5% compared to baseline. In some embodiments, after 52 weeks of treatment, the subject's percent-predicted FVC is increased by at least 0.1% compared to baseline. In some embodiments, compared to the control treatment, the subject's percent-predicted FVC is significantly improved after treatment. In some embodiments, compared to the control treatment, the subject's percent-predicted FVC is significantly improved by at least 1%, 2%, 3%, 4%, 5%, 6%, 8%, or 10% after 12, 26, 38, or 52 weeks of treatment. In some embodiments, compared to the control treatment, the subject's percent-predicted FVC is significantly improved by at least 4% after 52 weeks of treatment. In some embodiments, the subject has a baseline FVC less than 55%. In some embodiments, the subject has a baseline FVC greater than or equal to 55%.

In some embodiments, the two-component therapy for an ERT-experienced subject with Pompe disease improves the subject's motor function, as measured by a GSGC test. In some embodiments, compared to baseline, the subject's GSGC score is improved as indicated by a decrease of at least 0.1, 0.3, 0.5, 0.7, 1.0, 1.5, or 2.5 points after 12, 26, 38, or 52 weeks of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks of treatment. In some embodiments, compared to the control treatment, the subject's GSGC score is significantly improved as indicated by a decrease of at least 1.0 point after 52 weeks of treatment.

In some embodiments, the two-component therapy for an ERT-experienced subject with Pompe disease reduces the level of at least one marker of muscle damage after treatment. In some embodiments, the at least one marker of muscle damage comprises CK. In some embodiments, compared to baseline, the subject's CK level is reduced by at least 10%, 15%, 20%, 25%, 30%, 40%, or 50% after 12, 26, 38, or 52 weeks of treatment. In some embodiments, compared to baseline, the subject's CK level is reduced by at least 15% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks of treatment. In some embodiments, compared to the control treatment, the subject's CK level is significantly reduced by at least 30% after 52 weeks of treatment.

In some embodiments, the two-component therapy for an ERT-experienced subject with Pompe disease reduces the level of at least one marker of glycogen accumulation after treatment. In some embodiments, the at least one marker of glycogen accumulation comprises urinary Hex4. In some embodiments, compared to baseline, the subject's urinary Hex4 level is reduced by at least 10%, 15%, 20%, 25%, 30%, 40%, 50%, or 60% after 12, 26, 38, or 52 weeks of treatment. In some embodiments, compared to baseline, the subject's urinary Hex4 level is reduced by at least 25% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks of treatment. In some embodiments, compared to the control treatment, the subject's urinary Hex4 level is significantly reduced by at least 40% after 52 weeks of treatment.

### BRIEF DESCRIPTION OF THE DRAWINGS

FIG. 1A shows non-phosphorylated high mannose N-glycan, a mono-M6P N-glycan, and a bis-M6P N-glycan. FIG. 1B shows the chemical structure of the M6P group. Each square represents N-acetylglucosamine (GlcNAc), each circle represents mannose, and each P represents phosphate.

FIG. 2A describes productive targeting of rhGAA via N-glycans bearing M6P to target tissues (e.g., muscle tissues of subject with Pompe Disease). FIG. 2B describes non-productive drug clearance to non-target tissues (e.g., liver and spleen) or by binding of non-M6P N-glycans to non-target tissues.

FIG. 3 is a schematic diagram of an exemplary process for the manufacturing, capturing and purification of a recombinant lysosomal protein.

FIG. 4 shows a DNA construct for transforming CHO cells with DNA encoding rhGAA.

FIG. 5 is a graph showing the results of CIMPR affinity chromatography of ATB200 rhGAA with (Embodiment 2) and without (Embodiment 1) capture on an anion exchange (AEX) column.

FIG. 6A-FIG. 6H show the results of a site-specific N-glycosylation analysis of ATB200 rhGAA, using two different LC-MS/MS analytical techniques. FIG. 6A shows the site occupancy of the seven potential N-glycosylation sites for ATB200. FIG. 6B shows two analyses of the N-glycosylation profile of the first potential N-glycosylation site for ATB200. FIG. 6C shows two analyses of the N-glycosylation profile of the second potential N-glycosylation site for ATB200. FIG. 6D shows two analyses of the N-glycosylation profile of the third potential N-glycosylation site for ATB200. FIG. 6E shows two analyses of the N-glycosylation profile of the fourth potential N-glycosylation site for ATB200. FIG. 6F shows two analyses of the N-glycosylation profile of the fifth potential N-glycosylation site for ATB200. FIG. 6G shows two analyses of the N-glycosylation profile of the sixth potential N-glycosylation site for ATB200. FIG. 6H summarizes the relative percent mono-phosphorylated and bis-phosphorylated species for the first, second, third, fourth, fifth, and sixth potential N-glycosylation sites.

FIG. 7 is a graph showing Polywax elution profiles of LUMIZYME® (thinner line, eluting to the left) and ATB200 (thicker line, eluting to the right).

FIG. 8 is a table showing a summary of N-glycan structures of LUMIZYME® compared to three different preparations of ATB200 rhGAA, identified as BP-rhGAA, ATB200-1 and ATB200-2.

FIG. 9A and FIG. 9B are graphs showing the results of CIMPR affinity chromatography of LUMIZYME® and MYOZYME®, respectively.

FIG. 10A is a graph comparing the CIMPR binding affinity of ATB200 rhGAA (left trace) with that of LUMIZYME® (right trace). FIG. 10B is a table comparing the bis-M6P content of LUMIZYME® and ATB200 rhGAA.

FIG. 11A is a graph comparing ATB200 rhGAA activity (left trace) with LUMIZYME® rhGAA activity (right trace) inside normal fibroblasts at various GAA concentrations.

FIG. 11B is a table comparing ATB200 rhGAA activity (left trace) with LUMIZYME® rhGAA activity (right trace) inside fibroblasts from a subject having Pompe Disease at various GAA concentrations. FIG. 11C is a table comparing Kᵤₚₜₐₖₑ of fibroblasts from normal subjects and subjects with Pompe disease.

FIG. 12 depicts the stability of ATB200 in acidic or neutral pH buffers evaluated in a thermostability assay using SYPRO Orange, as the fluorescence of the dye increases when proteins denature.

FIG. 13 shows tissue glycogen content of WT mice or Gaa KO mice treated with a vehicle, alglucosidase alfa, or ATB200/AT2221, determined using amyloglucosidase digestion. Bars represent Mean±SEM of 7 mice/group. * p<0.05 compared to alglucosidase alfa in multiple comparison using Dunnett's method under one-way ANOVA analysis.

FIG. 14 depicts LAMP1-positive vesicles in muscle fibers of Gaa KO mice treated with a vehicle, alglucosidase alfa, or ATB200/AT2221 or WT mice. Images were taken from vastus lateralis and were representative of 7 mice per group. Magnification=200× (1,000× in insets).

FIG. 15A shows LC3-positive aggregates in muscle fibers of Gaa KO mice treated with a vehicle, alglucosidase alfa, or ATB200/AT2221 or WT mice. Images were taken from vastus lateralis and were representative of 7 mice per group. Magnification=400×. FIG. 15B shows a western blot analysis of LC3 II protein. A total of 30 mg protein was loaded in each lane.

FIG. 16 shows Dysferlin expression in muscle fibers of Gaa KO mice treated with a vehicle, alglucosidase alfa, or ATB200/AT2221 or WT mice. Images were taken from vastus lateralis and were representative of 7 mice per group. Magnification=200×.

FIG. 17 depicts co-immunofluorescent staining of LAMP1 (green) (see for example, “B”) and LC3 (red) (see, for example, “A”) in single fibers isolated from the white gastrocnemius of Gaa KO mice treated with a vehicle, alglucosidase alfa, or ATB200. “C” depicts clearance of autophagic debris and absence of enlarged lysosome. A minimum of 30 fibers were examined from each animal.

FIG. 18 depicts stabilization of ATB200 by AT2221 at 17 μM, and 170 μM AT2221, respectively, as compared to ATB200 alone.

FIG. 19A-FIG. 19H show the results of a site-specific N-glycosylation analysis of ATB200 rhGAA, including an N-glycosylation profile for the seventh potential N-glycosylation site, using LC-MS/MS analysis of protease-digested ATB200. FIG. 19A-FIG. 19H provide average data for ten lots of ATB200 produced at different scales.

FIG. 19A shows the average site occupancy of the seven potential N-glycosylation sites for ATB200. The N-glycosylation sites are provided according to SEQ ID NO: 1. CV=coefficient of variation.

FIG. 19B-FIG. 19H show the site-specific N-glycosylation analyses of all seven potential N-glycosylation sites for ATB200, with site numbers provided according to SEQ ID NO: 5. Bars represent the maximum and minimum percentage of N-glycan species identified as a particular N-glycan group for the ten lots of ATB200 analyzed. FIG. 19B shows the N-glycosylation profile of the first potential N-glycosylation site for ATB200. FIG. 19C shows the N-glycosylation profile of the second potential N-glycosylation site for ATB200. FIG. 19D shows the N-glycosylation profile of the third potential N-glycosylation site for ATB200. FIG. 19E shows the N-glycosylation profile of the fourth potential N-glycosylation site for ATB200. FIG. 19F shows the N-glycosylation profile of the fifth potential N-glycosylation site for ATB200. FIG. 19G shows the N-glycosylation profile of the sixth potential N-glycosylation site for ATB200. FIG. 19H shows the N-glycosylation profile of the seventh potential N-glycosylation site for ATB200.

FIG. 20A-FIG. 20B further characterize and summarize the N-glycosylation profile of ATB200, as also shown in FIGS. 19A-19H. FIG. 20A shows 2-Anthranilic acid (2-AA) glycan mapping and LC/MS-MS analysis of ATB200 and summarizes the N-glycan species identified in ATB200 as a percentage of total fluorescence. Data from 2-AA glycan mapping and LC-MS/MS analysis are also depicted in Table 5. FIG. 20B summarizes the average site occupancy and average N-glycan profile, including total phosphorylation, mono-phosphorylation, bis-phosphorylation, and sialylation, for all seven potential N-glycosylation sites for ATB200. ND=not detected.

FIG. 21 shows the ATB200-03 study design schematic.

FIG. 22 shows the baseline 6-minute walk distance (6MWD) and sitting forced vital capacity (FVC) characteristics of the 122 subjects who participated in the ATB200-03 study. AT-GAA group: subjects who received the ATB200/AT2221 treatment; Alglucosidase alfa group: subjects who received the alglucosidase alfa/placebo treatment.

FIG. 23A depicts the 6MWD and FVC data, showing the baseline, change from the baseline (“CFBL”) at week 52, difference, and P-value, for the overall population (n=122). AT-GAA group: subjects who received the ATB200/AT2221 treatment; Alglucosidase alfa group: subjects who received the alglucosidase alfa/placebo treatment.

FIG. 23B depicts the 6MWD and FVC data showing change from the baseline over time for the overall population (n=122). Cipaglucosidase alfa/miglustat group: subjects who received the ATB200/AT2221 treatment; Alglucosidase alfa/placebo: subjects who received the alglucosidase alfa/placebo treatment.

FIG. 24 depicts the 6MWD and FVC data, showing the baseline, CFBL at week 52, difference, and P-value, for the ERT-experienced population (n=95). AT-GAA group: subjects who received the ATB200/AT2221 treatment; Alglucosidase alfa group: subjects who received the alglucosidase alfa/placebo treatment.

FIG. 25 depicts the 6MWD and FVC changes relative to baseline at week 12, week 26, and week 38, and week 52, for the ERT-experienced population (n=95).

FIG. 26A depicts the 6MWD and FVC data, showing the baseline, CFBL at week 52, difference, and P-value, for the ERT-naïve population (n=27). AT-GAA group: subjects who received the ATB200/AT2221 treatment; Alglucosidase alfa group: subjects who received the alglucosidase alfa/placebo treatment.

FIG. 26B depicts the 6MWD and FVC data showing change from the baseline over time for the ERT-naïve population (n=27). Cipaglucosidase alfa/miglustat group: subjects who received the ATB200/AT2221 treatment; Alglucosidase alfa/placebo: subjects who received the alglucosidase alfa/placebo treatment.

FIG. 27 depicts baseline characteristics on key secondary endpoints and biomarkers for the overall and ERT-experienced populations. AT-GAA group: subjects who received the ATB200/AT2221 treatment; Alglucosidase alfa group: subjects who received the alglucosidase alfa/placebo treatment.

FIG. 28 depicts the lower manual muscle testing (MMT) changes relative to baseline at week 12, week 26, week 38, and week 52, for the overall population (left) and ERT-experienced population (right).

FIG. 29 depicts the gait, stairs, gowers, chair (GSGC) changes relative to baseline at week 12, week 26, week 38, and week 52, for the overall population (left) and ERT-experienced population (right). Cipaglucosidase alfa/miglustat group: subjects who received the ATB200/AT2221 treatment; Alglucosidase alfa/placebo: subjects who received the alglucosidase alfa/placebo treatment.

FIG. 30 depicts the patient-reported outcomes measurement information system (PROMIS) for physical function changes relative to baseline at week 12, week 26, week 38, and week 52, for the overall population (left) and ERT-experienced population (right).

FIG. 31 depicts the PROMIS for fatigue changes relative to baseline at week 12, week 26, week 38, and week 52, for the overall population (left) and ERT-experienced population (right).

FIG. 32 depicts the creatine kinase (CK) biomarker changes relative to baseline at week 12, week 26, week 38, and week 52, for the overall population (left) and ERT-experienced population (right).

FIG. 33 depicts the urine hexose tetrasaccharide (Hex4) biomarker changes relative to baseline at week 12, week 26, week 38, and week 52, for the overall population (left) and ERT-experienced population (right).

FIG. 34 shows the primary, secondary and biomarker endpoint heat map for the overall population (left) and ERT-experienced population (right). AT-GAA group: subjects who received the ATB200/AT2221 treatment; Alglucosidase alfa group: subjects who received the alglucosidase alfa/placebo treatment.

FIG. 35 summarizes the safety data from the ATB200-03 study. AT-GAA group: subjects who received the ATB200/AT2221 treatment; Alglucosidase alfa group: subjects who received the alglucosidase alfa/placebo treatment. TEAE: treatment emergent adverse event; IAR: infusion-associated reaction.

FIG. 36 summarizes results from the ATB200-03 study.

FIG. 37 describes the study objectives and statistical methods of the ATB200-03 study.

FIG. 38 describes the primary endpoint and secondary endpoints of the ATB200-03 study.

FIG. 39 summarizes the patient disposition of the ATB200-03 study.

FIG. 40 summarizes the baseline demographics of the ATB200-03 study.

FIG. 41A-FIG. 41B show subgroup analyses for the change from baseline in 6MWD and FVC by baseline status in the overall population (n=122) (FIG. 41A) and ERT-experienced patients (n=95) (FIG. 41B) in the ATB200-03 study.

FIG. 42 shows a list of treatment emergent adverse events (TEAEs) in ≥10% of patients in any group in the ATB200-03 study.

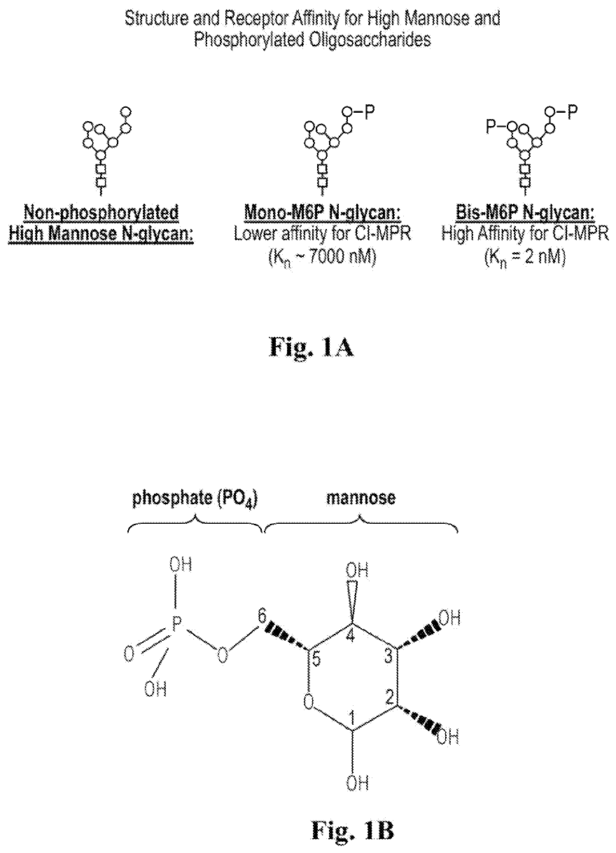

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Provided herein is a method for treating Pompe disease comprising administering to an individual a recombinant human α-glucosidase (rhGAA) and a pharmacological chaperone. The rhGAA has a higher total content of mannose-6-phosphate-bearing N-glycans, exhibits superior uptake into muscle cells and subsequent delivery to lysosomes compared to conventional rhGAA products, and possesses other pharmacokinetic properties that make it particularly effective for enzyme replacement therapy of subjects having Pompe disease. Accordingly, the two-component therapy according to this disclosure exhibits superior efficacy in treating and reversing disease progression in subjects suffering from Pompe disease compared to conventional therapies.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them. The articles “a” and “an” refer to one or to more than one (i.e., to at least one) of the grammatical object of the article. The term “or” means, and is used interchangeably with, the term “and/or,” unless context clearly indicates otherwise. In this application, the use of the singular includes the plural unless specifically stated otherwise. Furthermore, the use of the term “including,” as well as other forms, such as “includes” and “included,” are not limiting. Any range described herein will be understood to include the endpoints and all values between the endpoints. In the present specification, except where the context requires otherwise due to express language or necessary implication, the word “comprises”, or variations such as “comprising” is used in an inclusive sense, i.e., to specify the presence of the stated features but not to preclude the presence or addition of further features in various embodiments of the disclosure.

The term “GAA” refers to human acid α-glucosidase (GAA) enzyme that catalyzes the hydrolysis of α-1,4- and α-1,6-glycosidic linkages of lysosomal glycogen as well as to insertional, relational, or substitution variants of the GAA amino acid sequence and fragments of a longer GAA sequence that exert enzymatic activity. Human acid α-glucosidase is encoded by the GAA gene (National Centre for Biotechnology Information (NCBI) Gene ID 2548), which has been mapped to the long arm of chromosome 17 (location 17q25.2-q25.3). An exemplary amino acid sequence of GAA is NP 000143.2, which is incorporated by reference. This disclosure also encompasses DNA sequences that encode the amino acid sequence of NP 000143.2. More than 500 mutations have currently been identified in the human GAA gene, many of which are associated with Pompe disease. Mutations resulting in misfolding or misprocessing of the acid α-glucosidase enzyme include T1064C (Leu355Pro) and C2104T (Arg702Cys). In addition, GAA mutations which affect maturation and processing of the enzyme include Leu405Pro and Met519Thr. The conserved hexapeptide WIDMNE (SEQ ID NO: 7) at amino acid residues 516-521 is required for activity of the acid α-glucosidase protein. As used herein, the abbreviation “GAA” is intended to refer to human acid α-glucosidase enzyme, while the italicized abbreviation “GAA” is intended to refer to the human gene coding for the human acid α-glucosidase enzyme. The italicized abbreviation “Gaa” is intended to refer to non-human genes coding for non-human acid α-glucosidase enzymes, including but not limited to rat or mouse genes, and the abbreviation “Gaa” is intended to refer to non-human acid α-glucosidase enzymes.

The term “rhGAA” is intended to refer to the recombinant human acid α-glucosidase enzyme and is used to distinguish endogenous GAA from synthetic or recombinant-produced GAA (e.g., GAA produced from CHO cells or other host cells transformed with DNA encoding GAA). The term “rhGAA” encompasses a population of individual rhGAA molecules. Characteristics of the population of rhGAA molecules are provided herein. The term “conventional rhGAA product” is intended to refer to products containing alglucosidase alfa, such as LUMIZYME® or MYOZYME®.

The term “genetically modified” or “recombinant” refers to cells, such as CHO cells, that express a particular gene product, such as rhGAA, following introduction of a nucleic acid comprising a coding sequence which encodes the gene product, along with regulatory elements that control expression of the coding sequence. Introduction of the nucleic acid may be accomplished by any method known in the art including gene targeting and homologous recombination. As used herein, the term also includes cells that have been engineered to express or overexpress an endogenous gene or gene product not normally expressed by such cell, e.g., by gene activation technology.

As used herein, the term “alglucosidase alfa” is intended to refer to a recombinant human acid α-glucosidase identified as [199-arginine,223-histidine]prepro-α-glucosidase (human); Chemical Abstracts Registry Number 420794-05-0. Alglucosidase alfa is approved for marketing in the United States by Genzyme, as the products LUMIZYME® and MYOZYME®.

As used herein, the term “ATB200” is intended to refer to a recombinant human acid α-glucosidase described in U.S. Pat. No. 10,961,522, the disclosure of which is herein incorporated by reference. ATB200 is also referred to as “cipaglucosidase alfa”.

As used herein, the term “glycan” is intended to refer to an oligosaccharide covalently bound to an amino acid residue on a protein or polypeptide. As used herein, the term “N-glycan” or “N-linked glycan” is intended to refer to a polysaccharide chain attached to an asparagine residue on a protein or polypeptide through covalent binding to a nitrogen atom of the asparagine residue. In some embodiments, the N-glycan units attached to a rhGAA are determined by liquid chromatography-tandem mass spectrometry (LC-MS/MS) utilizing an instrument such as the Thermo Scientific™ Orbitrap Velos Pro™ Mass Spectrometer, Thermo Scientific™ Orbitrap Fusion™ Lumos Tribid™ Mass Spectrometer, or Waters Xevo® G2-XS QTof Mass Spectrometer.

As used herein, forced vital capacity, or “FVC,” is the amount of air that can be forcibly exhaled from the lungs of a subject after the subject takes the deepest breath possible.

As used herein, a “six-minute walk test” (6MWT) is a test for measuring the distance an individual is able to walk over a total of six minutes on a hard, flat surface. The test is conducted by having the individual to walk as far as possible in six minutes.

As used herein, a “ten-meter walk test” (10MWT) is a test for measuring the time it takes an individual in walking shoes to walk ten meters on a flat surface.

As used herein, the compound miglustat, also known as N-butyl-1-deoxynojirimycin or NB-DNJ or (2R,3R,4R,5S)-1-butyl-2-(hydroxymethyl)piperidine-3,4,5-triol, is a compound having the following chemical formula:

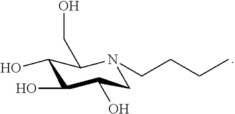

One formulation of miglustat is marketed commercially under the trade name ZAVESCA® as monotherapy for type 1 Gaucher disease. In some embodiments, miglustat is referred to as AT2221.

As discussed below, pharmaceutically acceptable salts of miglustat may also be used in the present disclosure. When a salt of miglustat is used, the dosage of the salt will be adjusted so that the dose of miglustat received by the patient is equivalent to the amount which would have been received had the miglustat free base been used.

As used herein, the compound duvoglustat, also known as 1-deoxynojirimycin or DNJ or (2R,3R,4R,5S)-2-(hydroxymethyl)piperidine-3,4,5-triol, is a compound having the following chemical formula:

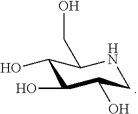

As used herein, the term “pharmacological chaperone” or sometimes simply the term “chaperone” is intended to refer to a molecule that specifically binds to acid α-glucosidase and has one or more of the following effects:
enhances the formation of a stable molecular conformation of the protein;
enhances proper trafficking of the protein from the endoplasmic reticulum to another cellular location, preferably a native cellular location, so as to prevent endoplasmic reticulum-associated degradation of the protein;
prevents aggregation of conformationally unstable or misfolded proteins;
restores and/or enhances at least partial wild-type function, stability, and/or activity of the protein; and/or
improves the phenotype or function of the cell harboring acid α-glucosidase.

Thus, a pharmacological chaperone for acid α-glucosidase is a molecule that binds to acid α-glucosidase, resulting in proper folding, trafficking, non-aggregation, and activity of acid α-glucosidase. In at least one embodiment, the pharmacological chaperone is miglustat. Another non-limiting example of a pharmacological chaperone for acid α-glucosidase is duvoglustat.

As used herein, the term “pharmaceutically acceptable” is intended to refer to molecular entities and compositions that are physiologically tolerable and do not typically produce untoward reactions when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. As used herein, the term “carrier” is intended to refer to a diluent, adjuvant, excipient, or vehicle with which a compound is administered. Suitable pharmaceutical carriers are known in the art and, in at least one embodiment, are described in “Remington's Pharmaceutical Sciences” by E. W. Martin, 18th Edition, or other editions.

The term “pharmaceutically acceptable salt” as used herein is intended to mean a salt which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Berge et al., J. Pharm. Sci., 1977, 66, pp. 1-19, herein incorporated by reference. The term “pharmaceutically-acceptable acid addition salt” as used herein is intended to mean those salts which retain the biological effectiveness and properties of the free bases and which are not biologically or otherwise undesirable, formed with inorganic acids. The term “pharmaceutically-acceptable base addition salt” as used herein is intended to mean those salts which retain the biological effectiveness and properties of the free acids and which are not biologically or otherwise undesirable, formed with inorganic bases.

As used herein, the term “buffer” refers to a solution containing a weak acid and its conjugate base or a weak base and its conjugate acid that helps to prevent changes in pH.

As used herein, the terms “therapeutically effective dose” and “effective amount” are intended to refer to an amount of acid α-glucosidase and/or of miglustat and/or of a two-component therapy thereof, which is sufficient to result in a therapeutic response in a subject.

The therapeutic response may also include molecular responses such as glycogen accumulation, lysosomal proliferation, and formation of autophagic zones. The therapeutic responses may be evaluated by comparing physiological and molecular responses of muscle biopsies before and after treatment with a rhGAA described herein. For instance, the amount of glycogen present in the biopsy samples can be used as a marker for determining the therapeutic response. Another example includes biomarkers such as LAMP-1, LC3, and Dysferlin, which can be used as an indicator of lysosomal storage dysfunction. For instance, muscle biopsies collected prior to and after treatment with a rhGAA described herein may be stained with an antibody that recognizes one of the biomarkers. The therapeutic response may also include a decrease in fatigue or improvement in other patient-reported outcomes (e.g., daily living activities, well-being, etc.).

As used herein, the term “enzyme replacement therapy” or “ERT” is intended to refer to the introduction of a non-native, purified enzyme into an individual having a deficiency in such enzyme. The administered protein can be obtained from natural sources or by recombinant expression. The term also refers to the introduction of a purified enzyme in an individual otherwise requiring or benefiting from administration of a purified enzyme. In at least one embodiment, such an individual suffers from enzyme insufficiency. The introduced enzyme may be a purified, recombinant enzyme produced in vitro, or a protein purified from isolated tissue or fluid, such as, for example, placenta or animal milk, or from plants.

As used herein, the term “two-component therapy” is intended to refer to any therapy wherein two or more individual therapies are administered concurrently or sequentially. In some embodiment, the results of the two-component therapy are enhanced as compared to the effect of each therapy when it is performed individually. Enhancement may include any improvement of the effect of the various therapies that may result in an advantageous result as compared to the results achieved by the therapies when performed alone. Enhanced effect or results can include a synergistic enhancement, wherein the enhanced effect is more than the additive effects of each therapy when performed by itself; an additive enhancement, wherein the enhanced effect is substantially equal to the additive effect of each therapy when performed by itself; or less than additive effect, wherein the enhanced effect is lower than the additive effect of each therapy when performed by itself, but still better than the effect of each therapy when performed by itself. Enhanced effect may be measured by any means known in the art by which treatment efficacy or outcome can be measured.

“Pompe disease” refers to an autosomal recessive LSD characterized by deficient acid alpha glucosidase (GAA) activity which impairs lysosomal glycogen metabolism. The enzyme deficiency leads to lysosomal glycogen accumulation and results in progressive skeletal muscle weakness, reduced cardiac function, respiratory insufficiency, and/or CNS impairment at late stages of disease. Genetic mutations in the GAA gene result in either lower expression or produce mutant forms of the enzyme with altered stability, and/or biological activity ultimately leading to disease, (see generally Hirschhorn R, 1995, Glycogen Storage Disease Type II: Acid α-Glucosidase (Acid Maltase) Deficiency, The Metabolic and Molecular Bases of Inherited Disease, Scriver et al., eds., McGraw-Hill, New York, 7th ed., pages 2443-2464). The three recognized clinical forms of Pompe Disease (infantile, juvenile, and adult) are correlated with the level of residual α-glucosidase activity (Reuser A J et al., 1995, Glycogenosis Type II (Acid Maltase Deficiency), Muscle & Nerve Supplement 3, S61-S69). Infantile Pompe disease (type I or A) is most common and most severe, characterized by failure to thrive, generalized hypotonic, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade (Felicia K J et al., 1995, Clinical Variability in Adult-Onset Acid Maltase Deficiency: Report of Affected Sibs and Review of the Literature, Medicine 74, 131-135). In Pompe disease, it has been shown that α-glucosidase is extensively modified post-translationally by glycosylation, phosphorylation, and proteolytic processing. Conversion of the 110 kilodalton (kDa) precursor to 76 and 70 KDa mature forms by proteolysis in the lysosome is required for optimum glycogen catalysis. As used herein, the term “Pompe disease” refers to all types of Pompe disease. The formulations and dosing regimens disclosed in this application may be used to treat, for example, Type I, Type II or Type III Pompe disease.

As used herein, “significant” refers to statistical significance. The term refers to statistical evidence that there is a difference between two treatment groups. It can be defined as the probability of making a decision to reject the null hypothesis when the null hypothesis is actually true. The decision is often made using a p-value<0.05 derived from a suitable statistical analysis for the comparison. See, e.g., Example 9.

A “subject” or “patient” is preferably a human, though other mammals and non-human animals having disorders involving accumulation of glycogen may also be treated. A subject may be a fetus, a neonate, child, juvenile, or an adult with Pompe disease or other glycogen storage or accumulation disorder. One example of an individual being treated is an individual (fetus, neonate, child, juvenile, adolescent, or adult human) having GSD-II (e.g., infantile GSD-II, juvenile GSD-II, or adult-onset GSD-II). The individual can have residual GAA activity, or no measurable activity. For example, the individual having GSD-II can have GAA activity that is less than about 1% of normal GAA activity (infantile GSD-II), GAA activity that is about 1-10% of normal GAA activity (juvenile GSD-II), or GAA activity that is about 10-40% of normal GAA activity (adult GSD-II). In some embodiments, the subject or patient is an “ERT-experienced” or “ERT-switch” patient, referring to a Pompe disease patient who has previously received enzyme replacement therapy. In some embodiments, an “ERT-experienced” or “ERT-switch” patient is a Pompe disease patient who has received or is currently receiving alglucosidase alfa for greater than or equal to 24 months. In some embodiments, the subject or patient is an “ERT-naïve” patient, referring to a Pompe disease patient who has not previously received enzyme replacement therapy. In certain embodiments, the subject or patient is ambulatory (e.g., an ambulatory ERT-switch patient or an ambulatory ERT-naïve patient). In certain embodiments, the subject or patient is nonambulatory (e.g., a nonambulatory ERT-switch patient). Ambulatory or nonambulatory status may be determined by a six-minute walk test (6MWT). In some embodiments, an ambulatory patient is a Pompe disease patient who is able to walk at least 200 meters in the 6MWT. In some embodiments, a nonambulatory patient is a Pompe disease patient who is unable to walk unassisted or who is wheelchair bound.

The terms “treat” and “treatment,” as used herein, refer to amelioration of one or more symptoms associated with the disease, delay of the onset of one or more symptoms of the disease, and/or lessening of the severity or frequency of one or more symptoms of the disease. For example, treatment can refer to improvement of cardiac status (e.g. increase of end-diastolic and/or end-systolic volumes, or reduction or amelioration of the progressive cardiomyopathy that is typically found in GSD-II) or of pulmonary function (e.g., increase in crying vital capacity over baseline capacity, and/or normalization of oxygen desaturation during crying); improvement in neurodevelopment and/or motor skills (e.g., increase in AIMS score); reduction of glycogen levels in tissue of the individual affected by the disease; or any combination of these effects. In one preferred embodiment, treatment includes improvement of cardiac status, particularly in reduction of GSD-II-associated cardiomyopathy.

The terms “improve,” “increase,” and “reduce,” as used herein, indicate values that are relative to a baseline measurement or the corresponding values from a control treatment, such as a measurement in the same individual prior to initiation of the treatment described herein, a measurement in a control individual (or multiple control individuals) in the absence of the treatment described herein, or a measurement after a control treatment. A control individual is an individual afflicted with the same form of GSD-II (either infantile, juvenile, or adult-onset) as the individual being treated, who is about the same age as the individual being treated (to ensure that the stages of the disease in the treated individual and the control individual(s) are comparable). In some embodiments, a control treatment comprises administering alglucosidase alfa and a placebo for a pharmacological chaperone (see Example 9).

As used herein, the terms “about” and “approximately” are intended to refer to an acceptable degree of error for the quantity measured given the nature or precision of the measurements. For example, the degree of error can be indicated by the number of significant figures provided for the measurement, as is understood in the art, and includes but is not limited to a variation of 1 in the most precise significant figure reported for the measurement. Typical exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

### II. Recombinant Human Acid α-Glucosidase (rhGAA)

In some embodiments, the recombinant human acid α-glucosidase (rhGAA) is an enzyme having an amino acid sequence as set forth in SEQ ID NO: 1, SEQ ID NO: 3, SEQ ID NO: 4, SEQ ID NO: 5, or SEQ ID NO: 6. In some embodiments, the rhGAA is encoded by a nucleotide sequence as set forth in SEQ ID NO: 2.

TABLE 1
Nucleotide Sequences and Protein Sequences

| SEQ ID | |
|---|---|
| NO: | Sequences |
| 1 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLOGA |
| | QMGQPWCFFPPSYPSYKLENLSSSEMGYTATLTRTTPTFFPKDILTLRLDVMMETENRLH |
| | FTIKDPANRRYEVPLETPRVHSRAPSPLYSVEFSEEPFGVIVHRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPI |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 2 | cagttgggaaagctgaggttgtcgccggggccgcgggtggaggtcggggatgaggcagcaggtaggac |
| | agtgacctcggtgacgcgaaggaccccggccacctctaggttctcctcgtccgcccgttgttcagcga |
| | gggaggctctgggcctgccgcagctgacggggaaactgaggcacggagcgggcctgtaggagctgtcc |
| | aggccatctccaaccatgggagtgaggcacccgccctgctcccaccggctcctggccgtctgcgccct |
| | cgtgtccttggcaaccgctgcactcctggggcacatcctactccatgatttcctgctggttccccgag |
| | agctgagtggctcctccccagtcctggaggagactcacccagctcaccagcagggagccagcagacca |
| | gggccccgggatgcccaggcacaccccggccgtcccagagcagtgcccacacagtgcgacgtcccccc |
| | caacagccgcttcgattgcgcccctgacaaggccatcacccaggaacagtgcgaggcccgcggctgct |
| | gctacatccctgcaaagcaggggctgcagggagcccagatggggcagccctggtgcttcttcccaccc |
| | agctaccccagctacaagctggagaacctgagctcctctgaaatgggctacacggccaccctgacccg |
| | taccacccccaccttcttccccaaggacatcctgaccctgcggctggacgtgatgatggagactgaga |
| | accgcctccacttcacgatcaaagatccagctaacaggcgctacgaggtgcccttggagaccccgcgt |
| | gtccacagccgggcaccgtccccactctacagcgtggagttctccgaggagcccttcggggtgatcgt |
| | gcaccggcagctggacggccgcgtgctgctgaacacgacggtggcgcccctgttctttgcggaccagt |
| | tccttcagctgtccacctcgctgccctcgcagtatatcacaggcctcgccgagcacctcagtcccctg |
| | atgctcagcaccagctggaccaggatcaccctgtggaaccgggaccttgcgcccacgcccggtgcgaa |
| | cctctacgggtctcaccctttctacctggcgctggaggacggcgggtcggcacacggggtgttcctgc |
| | taaacagcaatgccatggatgtggtcctgcagccgagccctgcccttagctggaggtcgacaggtggg |
| | atcctggatgtctacatcttcctgggcccagagcccaagagcgtggtgcagcagtacctggacgttgt |
| | gggatacccgttcatgccgccatactggggcctgggcttccacctgtgccgctggggctactcctcca |
| | ccgctatcacccgccaggtggtggagaacatgaccagggcccacttccccctggacgtccaatggaac |
| | gacctggactacatggactcccggagggacttcacgttcaacaaggatggcttccgggacttcccggc |
| | catggtgcaggagctgcaccagggcggccggcgctacatgatgatcgtggatcctgccatcagcagct |
| | cgggccctgccgggagctacaggccctacgacgagggtctgcggaggggggttttcatcaccaacgag |
| | accggccagccgctgattgggaaggtatggcccgggtccactgccttccccgacttcaccaaccccac |
| | agccctggcctggtgggaggacatggtggctgagttccatgaccaggtgcccttcgacggcatgtgga |
| | ttgacatgaacgagccttccaacttcatcagaggctctgaggacggctgccccaacaatgagctggag |
| | aacccaccctacgtgcctggggtggttggggggaccctccaggcggccaccatctgtgcctccagcca |
| | ccagtttctctccacacactacaacctgcacaacctctacggcctgaccgaagccatcgcctcccaca |
| | gggcgctggtgaaggctcgggggacacgcccatttgtgatctcccgctcgacctttgctggccacggc |
| | cgatacgccggccactggacgggggacgtgtggagctcctgggagcagctcgcctcctccgtgccaga |
| | aatcctgcagtttaacctgctgggggtgcctctggtcggggccgacgtctgcggcttcctgggcaaca |
| | cctcagaggagctgtgtgtgcgctggacccagctgggggccttctaccccttcatgcggaaccacaac |
| | agcctgctcagtctgccccaggagccgtacagcttcagcgagccggcccagcaggccatgaggaaggc |
| | cctcaccctgcgctacgcactcctcccccacctctacacactgttccaccaggcccacgtcgcggggg |
| | agaccgtggcccggcccctcttcctggagttccccaaggactctagcacctggactgtggaccaccag |
| | ctcctgtggggggaggccctgctcatcaccccagtgctccaggccgggaaggccgaagtgactggcta |
| | cttccccttgggcacatggtacgacctgcagacggtgccaatagaggcccttggcagcctcccacccc |
| | cacctgcagctccccgtgagccagccatccacagcgaggggcagtgggtgacgctgccggcccccctg |
| | gacaccatcaacgtccacctccgggctgggtacatcatccccctgcagggccctggcctcacaaccac |
| | agagtcccgccagcagcccatggccctggctgtggccctgaccaagggtggagaggcccgaggggagc |
| | tgttctgggacgatggagagagcctggaagtgctggagcgaggggcctacacacaggtcatcttcctg |
| | gccaggaataacacgatcgtgaatgagctggtacgtgtgaccagtgagggagctggcctgcagctgca |
| | gaaggtgactgtcctgggcgtggccacggcgccccagcaggtcctctccaacggtgtccctgtctcca |
| | acttcacctacagccccgacaccaaggtcctggacatctgtgtctcgctgttgatgggagagcagttt |
| | ctcgtcagctggtgttagccgggcggagtgtgttagtctctccagagggaggctggttccccagggaa |
| | gcagagcctgtgtgcgggcagcagctgtgtgcgggcctgggggttgcatgtgtcacctggagctgggc |
| | actaaccattccaagccgccgcatcgcttgtttccacctcctgggccggggctctggcccccaacgtg |
| | tctaggagagctttctccctagatcgcactgtgggccggggcctggagggctgctctgtgttaataag |
| | attgtaaggtttgccctcctcacctgttgccggcatgcgggtagtattagccacccccctccatctgt |
| | tcccagcaccggagaagggggtgctcaggtggaggtgtggggtatgcacctgagctcctgcttcgcgc |
| | ctgctgctctgccccaacgcgaccgcttcccggctgcccagagggctggatgcctgccggtccccgag |
| | caagcctgggaactcaggaaaattcacaggacttgggagattctaaatcttaagtgcaattattttaa |
| | taaaaggggcatttggaatc |
| 3 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLQGA |
| | QMGQPWCFFPPSYPSYKLENLSSSEMGYTATLTRTTPTFFPKDILTLRLDVMMETENRLH |
| | FTIKDPANRRYEVPLETPRVHSRAPSPLYSVEFSEEPFGVIVHRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPI |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 4 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLOGA |
| | QMGQPWCFFPPSYPSYKLENLSSSEMGYTATLTRTTPTFFPKDILTLRLDVMMETENRLH |
| | FTIKDPANRRYEVPLETPHVHSRAPSPLYSVEFSEEPFGVIVRRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPV |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 5 | QQGASRPGPRDAQAHPGRPRAVPTQCDVPPNSRFDCAPDKAITQEQCEARGCCYIPAKQG |
| | LQGAQMGQPWCFFPPSYPSYKLENLSSSEMGYTATLTRTTPTFFPKDILTLRLDVMMETE |
| | NRLHFTIKDPANRRYEVPLETPRVHSRAPSPLYSVEFSEEPFGVIVHRQLDGRVLLNTTV |
| | APLFFADQFLQLSTSLPSQYITGLAEHLSPLMLSTSWTRITLWNRDLAPTPGANLYGSHP |
| | FYLALEDGGSAHGVFLLNSNAMDVVLQPSPALSWRSTGGILDVYIFLGPEPKSVVQQYLD |
| | VVGYPFMPPYWGLGFHLCRWGYSSTAITRQVVENMTRAHFPLDVQWNDLDYMDSRRDFTF |
| | NKDGFRDFPAMVQELHQGGRRYMMIVDPAISSSGPAGSYRPYDEGLRRGVFITNETGQPL |
| | IGKVWPGSTAFPDFTNPTALAWWEDMVAEFHDQVPFDGMWIDMNEPSNFIRGSEDGCPNN |
| | ELENPPYVPGVVGGTLQAATICASSHQFLSTHYNLHNLYGLTEAIASHRALVKARGTRPF |
| | VISRSTFAGHGRYAGHWTGDVWSSWEQLASSVPEILQFNLLGVPLVGADVCGFLGNTSEE |
| | LCVRWTQLGAFYPFMRNHNSLLSLPQEPYSFSEPAQQAMRKALTLRYALLPHLYTLFHQA |
| | HVAGETVARPLFLEFPKDSSTWTVDHQLLWGEALLITPVLQAGKAEVTGYFPLGTWYDLQ |
| | TVPIEALGSLPPPPAAPREPAIHSEGQWVTLPAPLDTINVHLRAGYIIPLQGPGLTTTES |
| | RQQPMALAVALTKGGEARGELFWDDGESLEVLERGAYTQVIFLARNNTIVNELVRVTSEG |
| | AGLQLQKVTVLGVATAPQQVLSNGVPVSNFTYSPDTKVLDICVSLLMGEQFLVSWC |
| 6 | QQGASRPGPRDAQAHPGRPRAVPTQCDVPPNSRFDCAPDKAITQEQCEARGCCYIPAKQGLQGAQMGQ |
| | PWCFFPPSYPSYKLENLSSSEMGYTATLTRTTPTFFPKDILTLRLDVMMETENRLHFTIKDPANRRYE |
| | VPLETPHVHSRAPSPLYSVEFSEEPFGVIVRRQLDGRVLLNTTVAPLFFADQFLQLSTSLPSQYITGL |
| | AEHLSPLMLSTSWTRITLWNRDLAPTPGANLYGSHPFYLALEDGGSAHGVFLLNSNAMDVVLQPSPAL |
| | SWRSTGGILDVYIFLGPEPKSVVQQYLDVVGYPFMPPYWGLGFHLCRWGYSSTAITRQVVENMTRAHF |
| | PLDVQWNDLDYMDSRRDFTFNKDGFRDFPAMVQELHQGGRRYMMIVDPAISSSGPAGSYRPYDEGLRR |
| | GVFITNETGQPLIGKVWPGSTAFPDFTNPTALAWWEDMVAEFHDQVPFDGMWIDMNEPSNFIRGSEDG |
| | CPNNELENPPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVRWTQLGAFY |
| | PFMRNHNSLLSLPQEPYSFSEPAQQAMRKALTLRYALLPHLYTLFHQAHVAGETVARPLFLEFPKDSS |
| | TWTVDHQLLWGEALLITPVLQAGKAEVTGYFPLGTWYDLQTVPVEALGSLPPPPAAPREPAIHSEGQW |
| | VTLPAPLDTINVHLRAGYIIPLQGPGLTTTESRQQPMALAVALTKGGEARGELFWDDGESLEVLERGA |
| | YTQVIFLARNNTIVNELVRVTSEGAGLQLQKVTVLGVATAPQQVLSNGVPVSNFTYSPDTKVLDICVS |
| | LLMGEQFLVSWC |

In some embodiments, the rhGAA has a GAA amino acid sequence as set forth in SEQ ID NO: 1, as described in U.S. Pat. No. 8,592,362 and has GenBank accession number AHE24104.1 (GI:568760974). In some embodiments, the rhGAA has a GAA amino acid sequence as encoded in SEQ ID NO: 2, the mRNA sequence having GenBank accession number Y00839.1. In some embodiments, the rhGAA has a GAA amino acid sequence as set forth in SEQ ID NO: 3. In at some embodiments, the rhGAA has a GAA amino acid sequence as set forth in SEQ ID NO: 4, and has National Center for Biotechnology Information (NCBI) accession number NP_000143.2 or UniProtKB Accession Number P10253.

In some embodiments, the rhGAA is initially expressed as having the full-length 952 amino acid sequence of wild-type GAA as set forth in SEQ ID NO: 1 or SEQ ID NO: 4, and the rhGAA undergoes intracellular processing that removes a portion of the amino acids, e.g., the first 56 amino acids. Accordingly, the rhGAA that is secreted by the host cell can have a shorter amino acid sequence than the rhGAA that is initially expressed within the cell. In some embodiments, the shorter protein has the amino acid sequence set forth in SEQ ID NO: 5, which only differs from SEQ ID NO: 1 in that the first 56 amino acids comprising the signal peptide and precursor peptide have been removed, thus resulting in a protein having 896 amino acids. In some embodiments, the shorter protein has the amino acid sequence set forth in SEQ ID NO: 6, which only differs from SEQ ID NO: 4 in that the first 56 amino acids comprising the signal peptide and precursor peptide have been removed, thus resulting in a protein having 896 amino acids. Other variations in the number of amino acids are also possible, such as having 1, 2, 3, 4, 5, 6, 7, 8, 9, 10, 11, 12, 13, 14, 15, or more deletions, substitutions and/or insertions relative to the amino acid sequence described by SEQ ID NO: 1, SEQ ID NO: 4, SEQ ID NO: 5, or SEQ ID NO: 6. In some embodiments, the rhGAA product includes a mixture of recombinant human acid α-glucosidase molecules having different amino acid lengths.

In some embodiments, the rhGAA comprises an amino acid sequence that is at least 90%, 95%, 98% or 99% identical to SEQ ID NO: 4 or SEQ ID NO: 6. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

In some embodiments, the rhGAA undergoes post-translational and/or chemical modifications at one or more amino acid residues in the protein. For example, methionine and tryptophan residues can undergo oxidation. As another example, the N-terminal glutamine in SEQ ID NO: 6 can be further modified to form pyro-glutamate. As another example, asparagine residues can undergo deamidation to aspartic acid. As yet another example, aspartic acid residues can undergo isomerization to iso-aspartic acid. As yet another example, unpaired cysteine residues in the protein can form disulfide bonds with free glutathione and/or cysteine. Accordingly, in some embodiments, the enzyme is initially expressed as having an amino acid sequence as set forth in SEQ ID NO: 1, SEQ ID NO: 3, SEQ ID NO: 4, or SEQ ID NO: 5, or an amino acid sequence encoded by SEQ ID NO: 2, and the enzyme undergoes one or more of these post-translational and/or chemical modifications. Such modifications are also within the scope of the present disclosure.

### III. N-linked Glycosylation of rhGAA

There are seven potential N-linked glycosylation sites on a single rhGAA molecule. These potential glycosylation sites are at the following positions of SEQ ID NO: 6: N84, N177, N334, N414, N596, N826, and N869. Similarly, for the full-length amino acid sequence of SEQ ID NO: 4, these potential glycosylation sites are at the following positions: N140, N233, N390, N470, N652, N882, and N925. Other variants of rhGAA can have similar glycosylation sites, depending on the location of asparagine residues. Generally, sequences of Asn-X-Ser or Asn-X-Thr in the protein amino acid sequence indicate potential glycosylation sites, with the exception that X cannot be His or Pro.

The rhGAA molecules described herein may have, on average, 1, 2, 3, or 4 mannose-6-phosphate (M6P) groups on their N-glycans. For example, only one N-glycan on a rhGAA molecule may bear M6P (mono-phosphorylated or mono-M6P), a single N-glycan may bear two M6P groups (bis-phosphorylated or bis-M6P), or two different N-glycans on the same rhGAA molecule may each bear single M6P groups. In some embodiments, the rhGAA molecules described herein on average have 3-4 mol M6P groups on their N-glycans per mol rhGAA. Recombinant human acid α-glucosidase molecules may also have N-glycans bearing no M6P groups. In another embodiment, on average the rhGAA comprises greater than 2.5 mol M6P per mol rhGAA and greater than 4 mol sialic acid per mol rhGAA. In some embodiments, on average the rhGAA comprises about 3-3.5 mol M6P per mol rhGAA. In some embodiments, on average the rhGAA comprises about 4-5.4 mol sialic acid per mol rhGAA. On average at least about 3, 4, 5, 6, 7, 8, 9, 10%, or 20% of the total N-glycans on the rhGAA may be in the form of a mono-M6P N-glycan, for example, about 6.25% of the total N-glycans may carry a single M6P group and on average, at least about 0.5, 1, 1.5, 2.0, 2.5, 3.0% of the total N-glycans on the rhGAA are in the form of a bis-M6P N-glycan and on average less than 25% of total rhGAA contains no phosphorylated N-glycan binding to CIMPR. In some embodiments, on average about 10% to about 14% of the total N-glycans on the rhGAA are mono-phosphorylated. In some embodiments, on average about 7% to about 25% of the total N-glycans on the rhGAA are bis-phosphorylated. In some embodiments, on average the rhGAA comprises about 1.3 mol bis-M6P per mol rhGAA.

The rhGAA described herein may have on average from 0.5 to 7.0 mol M6P per mol rhGAA or any intermediate value or subrange thereof including 0.5, 1.0, 1.5, 2.0, 2.5, 3.0, 3.5, 4.0, 4.5, 5.0, 5.5, 6.0, 6.5, or 7.0 mol M6P per mol rhGAA. The rhGAA can be fractionated to provide rhGAA preparations with different average numbers of mono-M6P-bearing or bis-M6P-bearing N-glycans, thus permitting further customization of rhGAA targeting to the lysosomes in target tissues by selecting a particular fraction or by selectively combining different fractions.

In some embodiments, up to 60% of the N-glycans on the rhGAA may be fully sialylated, for example, up to 10%, 20%, 30%, 40%, 50% or 60% of the N-glycans may be fully sialylated. In some embodiments, no more than 50% of the N-glycans on the rhGAA are fully sialylated. In some embodiments, from 4% to 20% of the total N-glycans are fully sialylated. In other embodiments, no more than 5%, 10%, 20% or 30% of N-glycans on the rhGAA carry sialic acid and a terminal galactose residue (Gal). This range includes all intermediate values and subranges, for example, 7% to 30% of the total N-glycans on the rhGAA can carry sialic acid and terminal galactose. In yet other embodiments, no more than 5%, 10%, 15%, 16%, 17%, 18%, 19%, or 20% of the N-glycans on the rhGAA have a terminal galactose only and do not contain sialic acid. This range includes all intermediate values and subranges, for example, from 8% to 19% of the total N-glycans on the rhGAA in the composition may have terminal galactose only and do not contain sialic acid.

In some embodiments, 40% to 60%, 45% to 60%, 50% to 60%, or 55% to 60% of the total N-glycans on the rhGAA are complex type N-glycans; or no more than 1%, 2%, 3%, 4%, 5%, 6,%, or 7% of total N-glycans on the rhGAA are hybrid-type N-glycans; no more than 5%, 10%, 15%, 20%, or 25% of the high mannose-type N-glycans on the rhGAA are non-phosphorylated; at least 5% or 10% of the high mannose-type N-glycans on the rhGAA are mono-phosphorylated; and/or at least 1% or 2% of the high mannose-type N-glycans on the rhGAA are bis-phosphorylated. These values include all intermediate values and subranges. A rhGAA may meet one or more of the content ranges described above.

In some embodiments, the rhGAA may bear, on average, 2.0 to 8.0 moles of sialic acid residues per mole of rhGAA. This range includes all intermediate values and subranges thereof, including 2.0, 2.5, 3.0, 3.5, 4.0, 4.5, 5.0, 5.5, 6.0, 6.5, 7.0, 7.5, and 8.0 mol sialic acid residues per mol rhGAA. Without being bound by theory, it is believed that the presence of N-glycan units bearing sialic acid residues may prevent non-productive clearance of the rhGAA by asialoglycoprotein receptors.

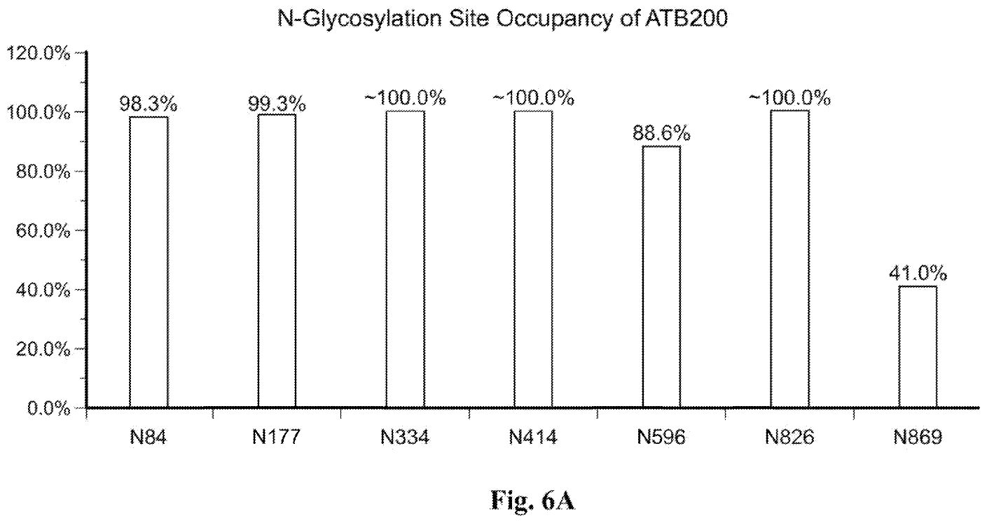

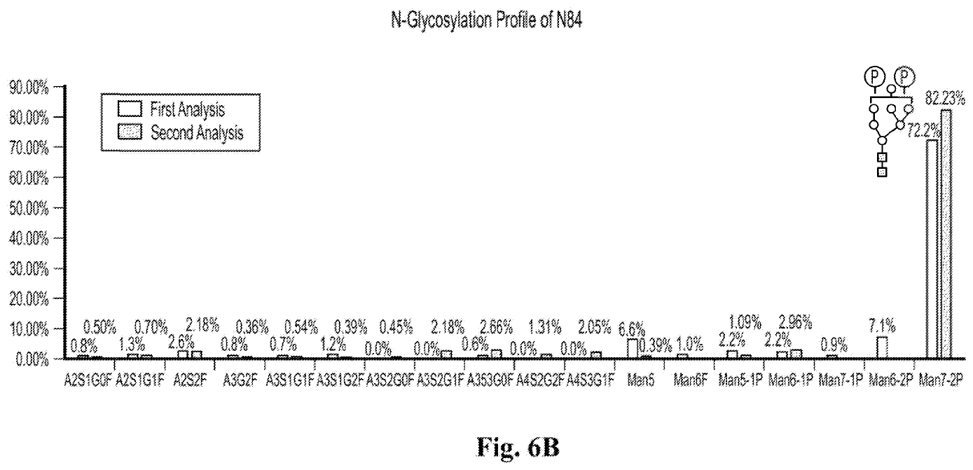

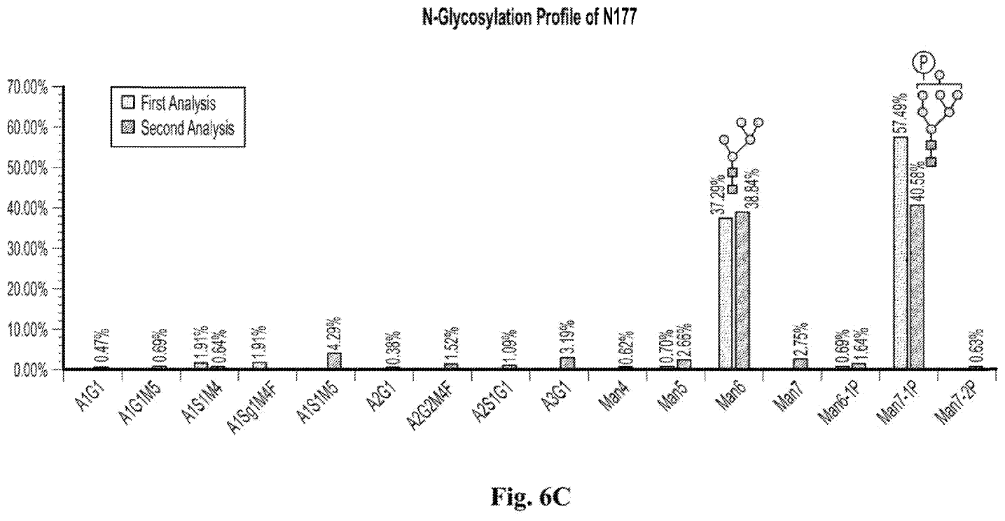

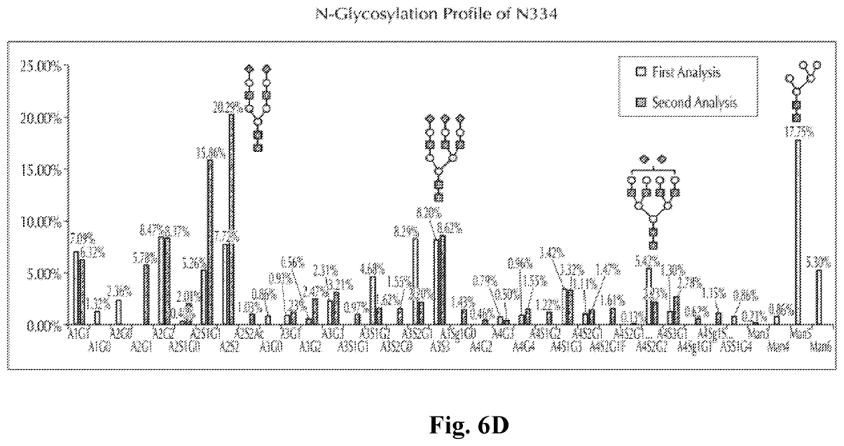

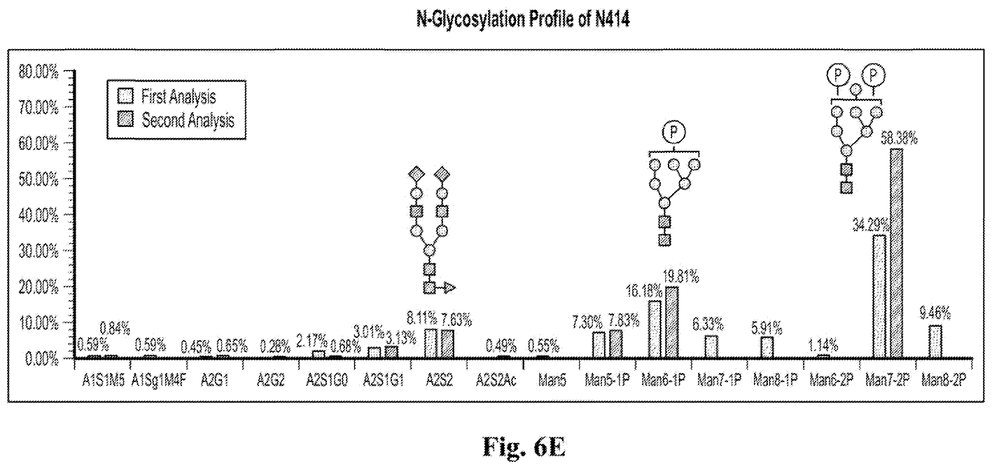

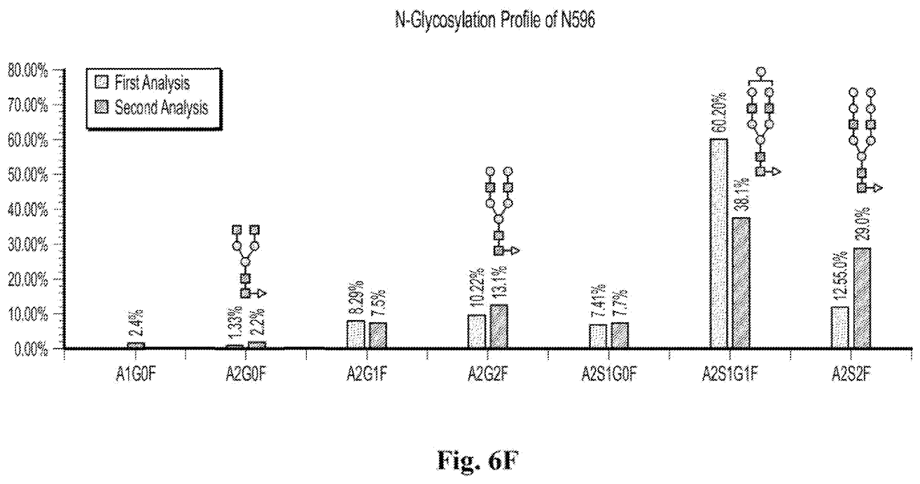

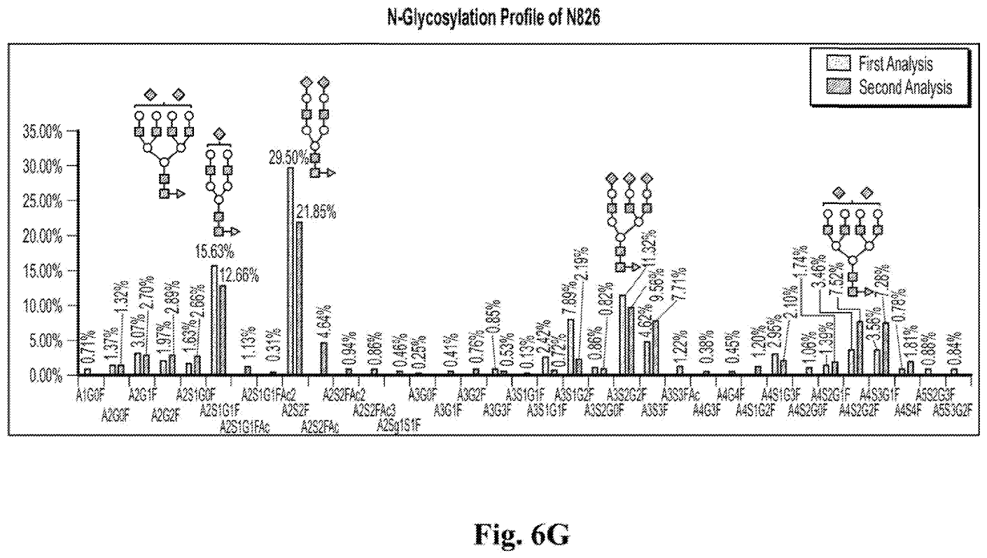

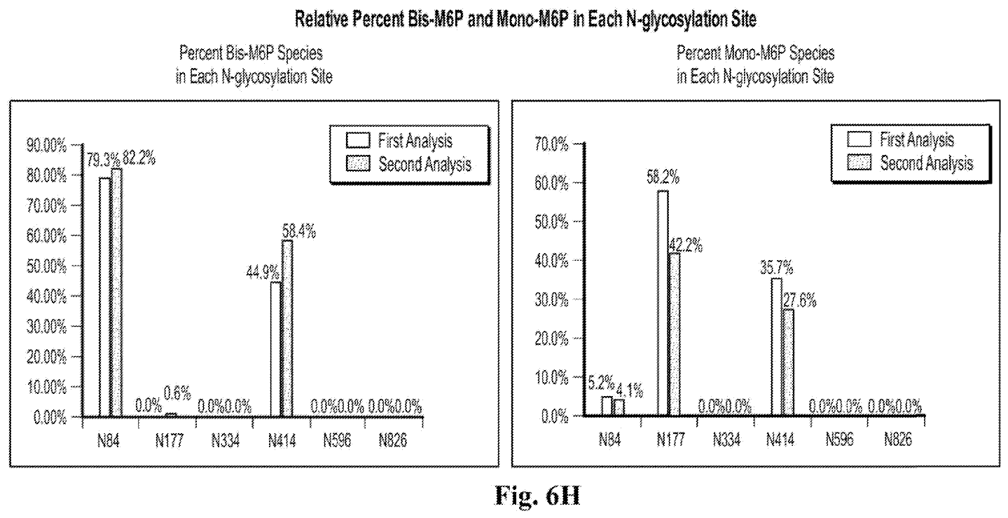

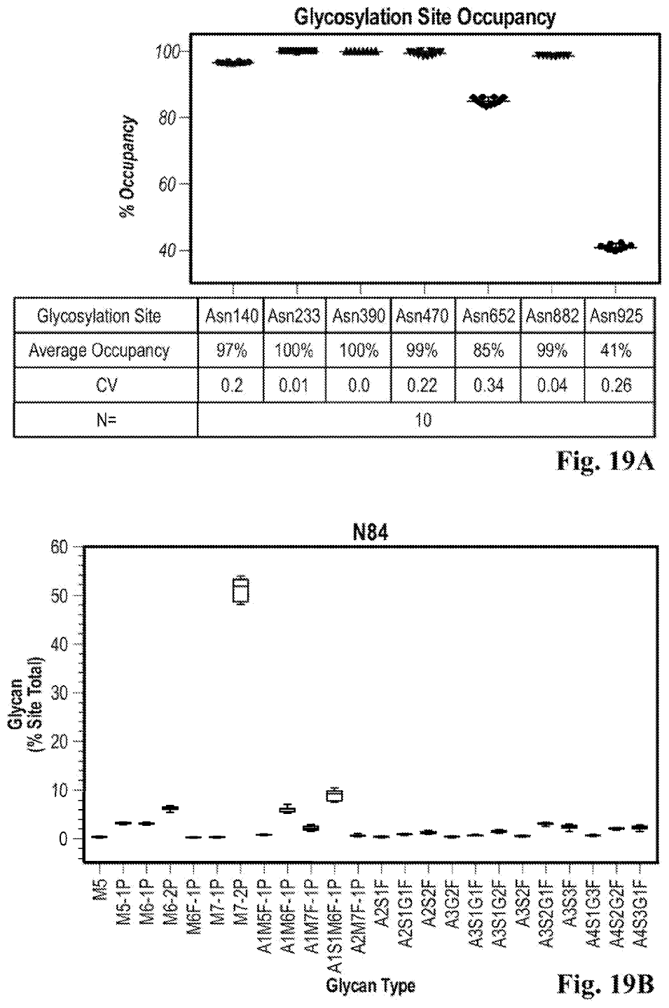

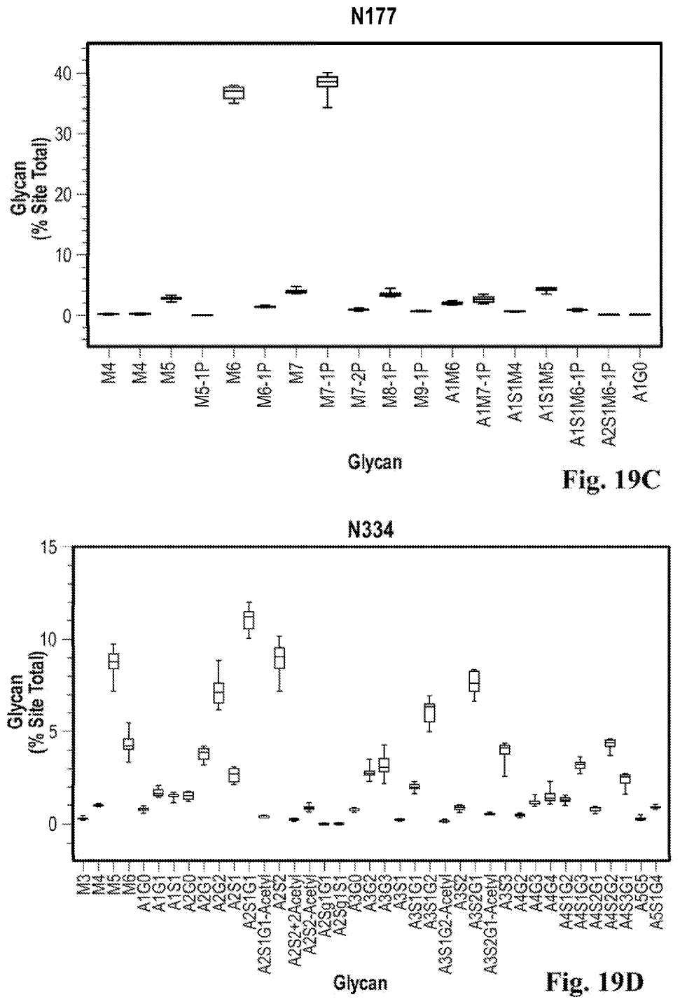

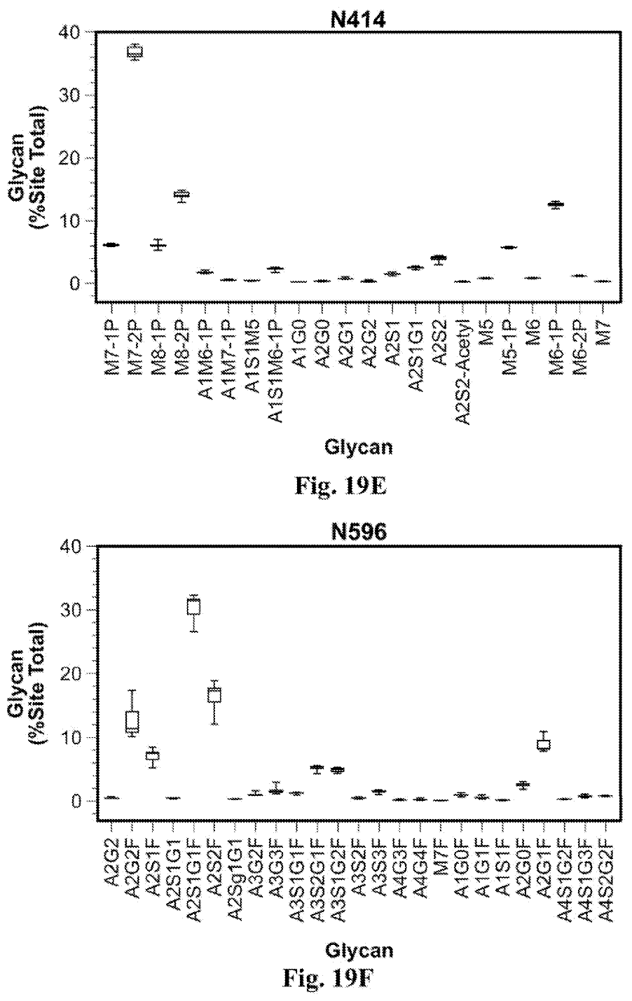

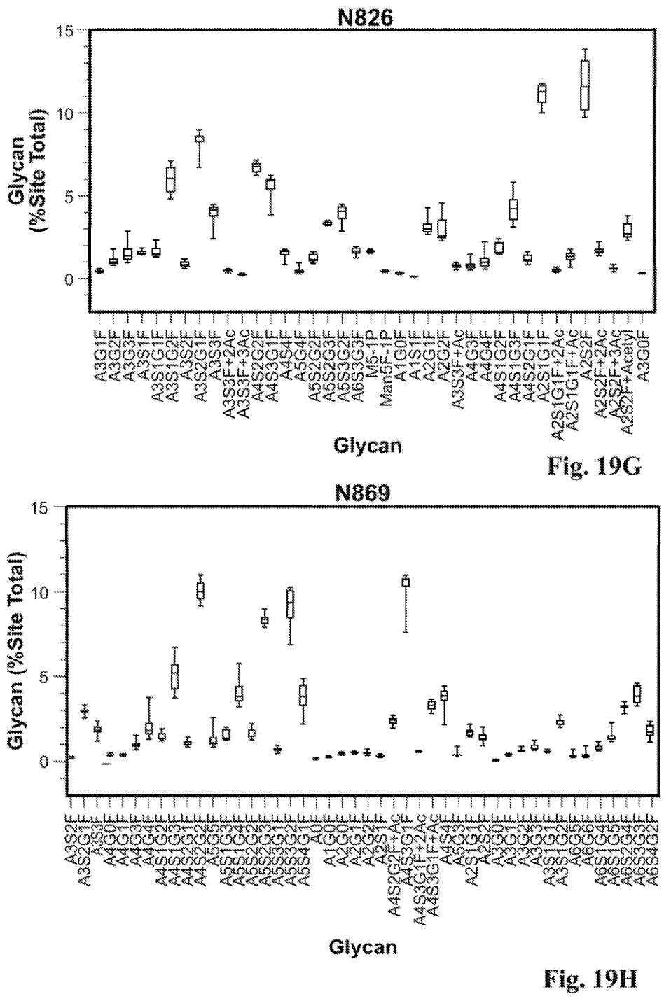

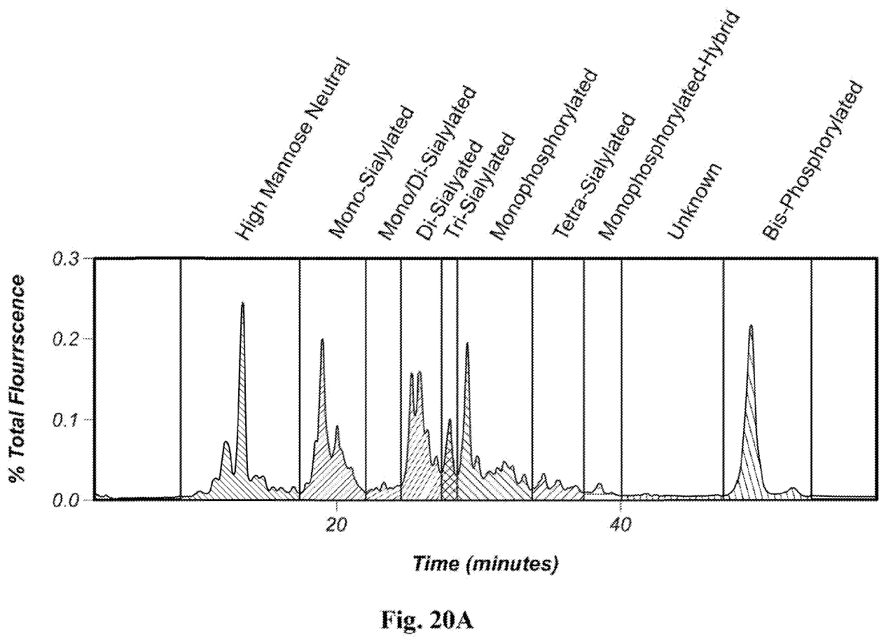

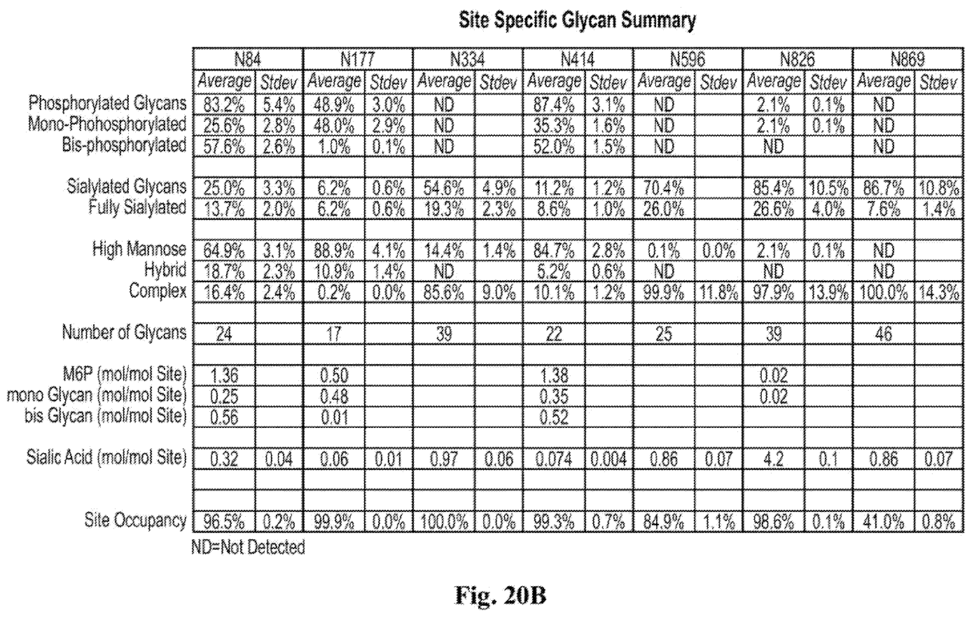

In one or more embodiments, the rhGAA has a certain N-glycosylation profile at certain potential N-glycosylation sites. In some embodiments, the rhGAA has seven potential N-glycosylation sites. In some embodiments, at least 20% of the rhGAA is phosphorylated at the first potential N-glycosylation site (e.g., N84 for SEQ ID NO: 6 and N140 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the first potential N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the first potential N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the first potential N-glycosylation site. In some embodiments, the rhGAA comprises on average about 1.4 mol M6P (mono-M6P and bis-M6P) per mol rhGAA at the first potential N-glycosylation site. In some embodiments, the rhGAA comprises on average about at least 0.5 mol bis-M6P per mol rhGAA at the first potential N-glycosylation site. In some embodiments, the rhGAA comprises on average about 0.25 mol mono-M6P per mol rhGAA at the first potential N-glycosylation site. In some embodiments, the rhGAA comprises on average about 0.2 mol to about 0.3 mol sialic acid per mol rhGAA at the first potential N-glycosylation site. In at least one embodiment, the rhGAA comprises a first potential N-glycosylation site occupancy as depicted in FIG. 6A and an N-glycosylation profile as depicted in FIG. 6B. In at least one embodiment, the rhGAA comprises a first potential N-glycosylation site occupancy as depicted in FIG. 19A and an N-glycosylation profile as depicted in FIG. 19B or FIG. 20B.

In some embodiments, at least 20% of the rhGAA is phosphorylated at the second potential N-glycosylation site (e.g., N177 for SEQ ID NO: 6 and N223 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the second N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the second N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the second N-glycosylation site. In some embodiments, the rhGAA comprises on average about 0.5 mol M6P (mono-M6P and bis-M6P) per mol rhGAA at the second potential N-glycosylation site. In some embodiments, the rhGAA comprises on average about 0.4 to about 0.6 mol mono-M6P per mol rhGAA at the second potential N-glycosylation site. In at least one embodiment, the rhGAA comprises a second potential N-glycosylation site occupancy as depicted in FIG. 6A and an N-glycosylation profile as depicted in FIG. 6C. In at least one embodiment, the rhGAA comprises a second potential N-glycosylation site occupancy as depicted in FIG. 19A and an N-glycosylation profile as depicted in FIG. 19C or FIG. 20B.

In one or more embodiments, at least 5% of the rhGAA is phosphorylated at the third potential N-glycosylation site (e.g., N334 for SEQ ID NO: 6 and N390 for SEQ ID NO: 4). In other embodiments, less than 5%, 10%, 15%, 20%, or 25% of the rhGAA is phosphorylated at the third potential N-glycosylation site. For example, the third potential N-glycosylation site can have a mixture of non-phosphorylated high mannose N-glycans, di-, tri-, and tetra-antennary complex N-glycans, and hybrid N-glycans as the major species. In some embodiments, at least 3%, 5%, 8%, 10%, 15%, 20%, 25%, 30%, 35%, 40%, 45%, or 50% of the rhGAA is sialylated at the third potential N-glycosylation site. In some embodiments, the rhGAA comprises on average about 0.9 to about 1.2 mol sialic acid per mol rhGAA at the third potential N-glycosylation site. In at least one embodiment, the rhGAA comprises a third potential N-glycosylation site occupancy as depicted in FIG. 6A and an N-glycosylation profile as depicted in FIG. 6D. In at least one embodiment, the rhGAA comprises a third potential N-glycosylation site occupancy as depicted in FIG. 19A and an N-glycosylation profile as depicted in FIG. 19D or FIG. 20B.

In some embodiments, at least 20% of the rhGAA is phosphorylated at the fourth potential N-glycosylation site (e.g., N414 for SEQ ID NO: 6 and N470 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the fourth potential N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the fourth potential N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the fourth potential N-glycosylation site. In some embodiments, at least 3%, 5%, 8%, 10%, 15%, 20%, or 25% of the rhGAA is sialylated at the fourth potential N-glycosylation site. In some embodiments, the rhGAA comprises on average about 1.4 mol M6P (mono-M6P and bis-M6P) per mol rhGAA at the fourth potential N-glycosylation site. In some embodiments, the rhGAA comprises on average about 0.4 to about 0.6 mol bis-M6P per mol rhGAA at the fourth potential N-glycosylation site. In some embodiments, the rhGAA comprises on average about 0.3 to about 0.4 mol mono-M6P per mol rhGAA at the fourth potential N-glycosylation site. In at least one embodiment, the rhGAA comprises a fourth potential N-glycosylation site occupancy as depicted in FIG. 6A and an N-glycosylation profile as depicted in FIG. 6E. In at least one embodiment, the rhGAA comprises a fourth potential N-glycosylation site occupancy as depicted in FIG. 19A and an N-glycosylation profile as depicted in FIG. 19E or FIG. 20B.

In some embodiments, at least 5% of the rhGAA is phosphorylated at the fifth potential N-glycosylation site (e.g., N596 for SEQ ID NO: 6 and N692 for SEQ ID NO: 4). In other embodiments, less than 5%, 10%, 15%, 20%, or 25% of the rhGAA is phosphorylated at the fifth potential N-glycosylation site. For example, the fifth potential N-glycosylation site can have fucosylated di-antennary complex N-glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90%, or 95% of the rhGAA is sialylated at the fifth potential N-glycosylation site. In some embodiments, the rhGAA comprises on average about 0.8 to about 0.9 mol sialic acid per mol rhGAA at the fifth potential N-glycosylation site. In at least one embodiment, the rhGAA comprises a fifth potential N-glycosylation site occupancy as depicted in FIG. 6A and an N-glycosylation profile as depicted in FIG. 6F. In at least one embodiment, the rhGAA comprises a fifth potential N-glycosylation site occupancy as depicted in FIG. 19A and an N-glycosylation profile as depicted in FIG. 19F or FIG. 20B.

In some embodiments, at least 5% of the rhGAA is phosphorylated at the sixth N-glycosylation site (e.g., N826 for SEQ ID NO: 6 and N882 for SEQ ID NO: 4). In other embodiments, less than 5%, 10%, 15%, 20% or 25% of the rhGAA is phosphorylated at the sixth N-glycosylation site. For example, the sixth N-glycosylation site can have a mixture of di-, tri-, and tetra-antennary complex N-glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90% or 95% of the rhGAA is sialylated at the sixth N-glycosylation site. In some embodiments, the rhGAA comprises on average about 1.5 to about 4.2 mol sialic acid per mol rhGAA at the sixth potential N-glycosylation site. In some embodiments, the rhGAA comprises on average about 0.9 mol acetylated sialic acid per mol rhGAA at the sixth potential N-glycosylation site. In some embodiments, the rhGAA comprises an average of at least 0.05 mol glycan species with poly-N-Acetyl-D-lactosamine (poly-LacNAc) residues per mol rhGAA at the sixth potential N-glycosylation site. In some embodiments, over 10% of the rhGAA comprises a glycan bearing a poly-LacNAc residue at the sixth potential N-glycosylation site. In at least one embodiment, the rhGAA comprises a sixth potential N-glycosylation site occupancy as depicted in FIG. 6A and an N-glycosylation profile as depicted in FIG. 6G. In at least one embodiment, the rhGAA comprises a sixth potential N-glycosylation site occupancy as depicted in FIG. 19A and an N-glycosylation profile as depicted in FIG. 19G or FIG. 20B.

In some embodiments, at least 5% of the rhGAA is phosphorylated at the seventh potential N-glycosylation site (e.g., N869 for SEQ ID NO: 6 and N925 for SEQ ID NO: 4). In other embodiments, less than 5%, 10%, 15%, 20%, or 25% of the rhGAA is phosphorylated at the seventh potential N-glycosylation site. In some embodiments, less than 40%, 45%, 50%, 55%, 60%, or 65% of the rhGAA has any N-glycan at the seventh potential N-glycosylation site. In some embodiments, at least 30%, 35%, or 40% of the rhGAA has an N-glycan at the seventh potential N-glycosylation site. In some embodiments, the rhGAA comprises on average at least 0.5 mol sialic acid per mol rhGAA at the seventh potential N-glycosylation site. In some embodiments, the rhGAA comprises on average at least 0.8 mol sialic acid per mol rhGAA at the seventh potential N-glycosylation site. In some embodiments, the rhGAA comprises on average about 0.86 mol sialic acid per mol rhGAA at the seventh potential N-glycosylation site. In some embodiments, the rhGAA comprises an average of at least 0.3 mol glycan species bearing poly-LacNAc residues per mol rhGAA at the seventh potential N-glycosylation site. In some embodiments, nearly half of the rhGAA comprises a glycan bearing a poly-LacNAc residue at the seventh potential N-glycosylation site. In at least one embodiment, all N-glycans identified at the seventh potential N-glycosylation site are complex N-glycans. In at least one embodiment, the rhGAA comprises a seventh potential N-glycosylation site occupancy as depicted in FIG. 6A or as depicted in FIG. 19A and an N-glycosylation profile as depicted in FIG. 19H or FIG. 20B.

In some embodiments, the rhGAA comprises on average 3-4 mol M6P residues per mol rhGAA and about 4 to about 7.3 mol sialic acid per mol rhGAA. In some embodiments, the rhGAA further comprises on average at least about 0.5 mol bis-M6P per mol rhGAA at the first potential N-glycosylation site, about 0.4 to about 0.6 mol mono-M6P per mol rhGAA at the second potential N-glycosylation site, about 0.9 to about 1.2 mol sialic acid per mol rhGAA at the third potential N-glycosylation site, about 0.4 to about 0.6 mol bis-M6P per mol rhGAA at the fourth potential N-glycosylation site, about 0.3 to about 0.4 mol mono-M6P per mol rhGAA at the fourth potential N-glycosylation site, about 0.8 to about 0.9 mol sialic acid per mol rhGAA at the fifth potential N-glycosylation site, and about 1.5 to about 4.2 mol sialic acid per mol rhGAA at the sixth potential N-glycosylation site. In some embodiments, the rhGAA further comprises on average at least 0.5 mol sialic acid per mol rhGAA at the seventh potential N-glycosylation site. In some embodiments, the rhGAA comprises on average at least 0.8 mol sialic acid per mol rhGAA at the seventh potential N-glycosylation site. In at least one embodiment, the rhGAA further comprises on average about 0.86 mol sialic acid per mol rhGAA at the seventh potential N-glycosylation site. In at least one embodiment, the rhGAA comprises seven potential N-glycosylation sites with occupancy and N-glycosylation profiles as depicted in FIGS. 6A-6H. In at least one embodiment, the rhGAA comprises seven potential N-glycosylation sites with occupancy and N-glycosylation profiles as depicted in FIGS. 19A-19H and FIGS. 20A-20B.

Methods of making rhGAA are disclosed in U.S. Provisional Patent Application No. 62/057,842, filed Sep. 30, 2014, the entire content of which is incorporated herein by reference.

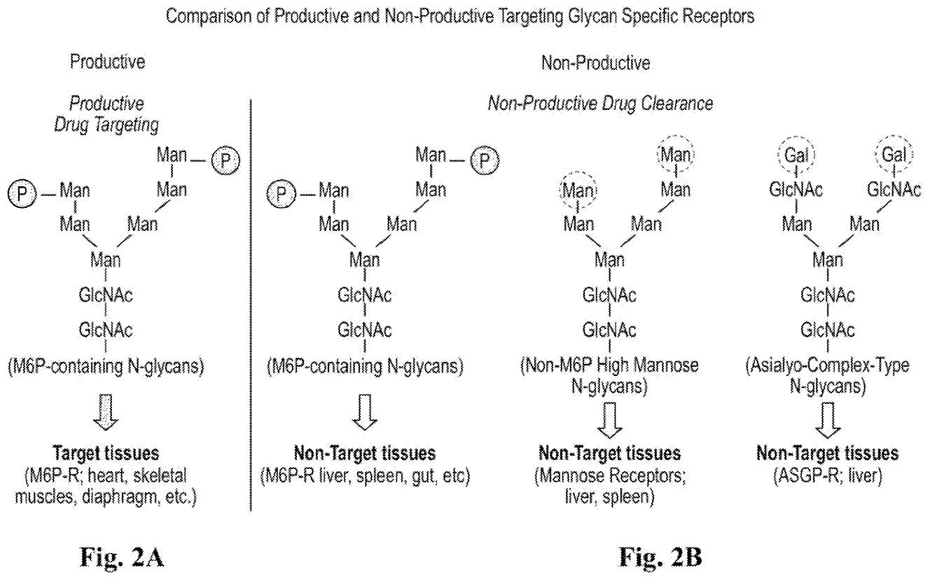

Once inside the lysosome, rhGAA can enzymatically degrade accumulated glycogen. However, conventional rhGAA products have low total levels of mono-M6P- and bis-M6P bearing N-glycans and, thus, target muscle cells poorly, resulting in inferior delivery of rhGAA to the lysosomes. The majority of rhGAA molecules in these conventional products do not have phosphorylated N-glycans, thereby lacking affinity for the CIMPR. Non-phosphorylated high mannose N-glycans can also be cleared by the mannose receptor, which results in non-productive clearance of the ERT (FIG. 2B). In contrast, as shown in FIG. 2A, a rhGAA described herein may contains a higher amount of mono-M6P- and bis-M6P bearing N-glycans, leading to productive uptake of rhGAA into specific tissues such as muscle.

### IV. Production and Purification of N-Linked Glycosylated rhGAA

As described in U.S. Pat. No. 10,961,522, the entirety of which is incorporated herein by reference, cells such as Chinese hamster ovary (CHO) cells may be used to produce the rhGAA described therein. Expressing high M6P rhGAA in CHO cells is advantageous over modifying the glycan profile of an rhGAA post-translationally at least in part because only the former may be converted by glycan degradation to a form of rhGAA with optimal glycogen hydrolysis, thus enhancing therapeutic efficacy.

In some embodiments, the rhGAA is preferably produced by one or more CHO cell lines that are transformed with a DNA construct encoding the rhGAA described herein. Such CHO cell lines may contain multiple copies of a gene, such as 5, 10, 15, or 20 or more copies, of a polynucleotide encoding GAA. DNA constructs, which express allelic variants of acid α-glucosidase or other variant acid α-glucosidase amino acid sequences such as those that are at least 90%, 95%, 98%, or 99% identical to SEQ ID NO: 4 or SEQ ID NO: 6, may be constructed and expressed in CHO cells. Those of skill in the art may select alternative vectors suitable for transforming CHO cells for production of such DNA constructs.

Methods for making such CHO cell lines are described in U.S. Pat. No. 10,961,522, the entirety of which is incorporated herein by reference. Briefly, these methods involve transforming a CHO cell with DNA encoding GAA or a GAA variant, selecting a CHO cell that stably integrates the DNA encoding GAA into its chromosome(s) and that stably expresses GAA, and selecting a CHO cell that expresses GAA having a high content of N-glycans bearing mono-M6P or bis-M6P, and, optionally, selecting a CHO cell having N-glycans with high sialic acid content and/or having N-glycans with a low non-phosphorylated high-mannose content. The selected CHO cell lines may be used to produce rhGAA and rhGAA compositions by culturing the CHO cell line and recovering said composition from the culture of CHO cells. In some embodiments, a rhGAA produced from the selected CHO cell lines contains a high content of N-glycans bearing mono-M6P or bis-M6P that target the CIMPR. In some embodiments, a rhGAA produced as described herein has low levels of complex N-glycans with terminal galactose. In some embodiments, the selected CHO cell lines are referred to as GA-ATB200 or ATB200-X5-14. In some embodiments, the selected CHO cell lines encompass a subculture or derivative of such a CHO cell culture. In some embodiments, a rhGAA produced from the selected CHO cell lines is referred to as ATB200.

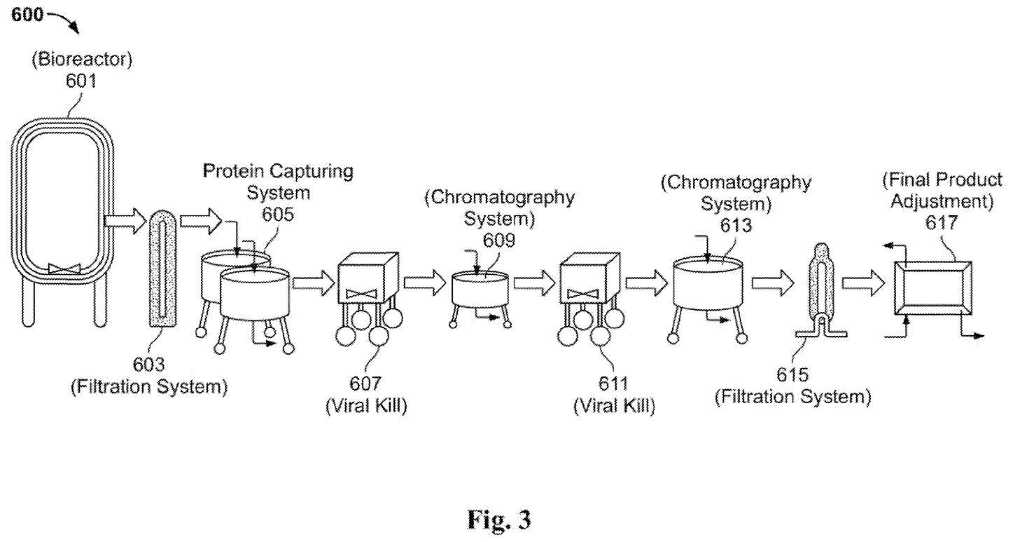

A rhGAA produced as described herein may be purified by following methods described in U.S. Pat. No. 10,227,577 and in U.S. Provisional Application No. 62/506,569, both of which are incorporated herein by reference in their entirety. An exemplary process for producing, capturing, and purifying a rhGAA produced from CHO cell lines is shown in FIG. 3.

Briefly, bioreactor 601 contains a culture of cells, such as CHO cells, that express and secrete rhGAA into the surrounding liquid culture media. The bioreactor 601 may be any appropriate bioreactor for culturing the cells, such as a perfusion, batch or fed-batch bioreactor. The culture media is removed from the bioreactor after a sufficient period of time for cells to produce rhGAA. Such media removal may be continuous for a perfusion bioreactor or may be batch-wise for a batch or fed-batch reactor. The media may be filtered by filtration system 603 to remove cells. Filtration system 603 may be any suitable filtration system, including an alternating tangential flow filtration (ATF) system, a tangential flow filtration (TFF) system, and/or centrifugal filtration system. In various embodiments, the filtration system utilizes a filter having a pore size between about 10 nanometers and about 2 micrometers.

After filtration, the filtrate is loaded onto a protein capturing system 605. The protein capturing system 605 may include one or more chromatography columns. If more than one chromatography column is used, then the columns may be placed in series so that the next column can begin loading once the first column is loaded. Alternatively, the media removal process can be stopped during the time that the columns are switched.

In various embodiments, the protein capturing system 605 includes one or more anion exchange (AEX) columns for the direct product capture of rhGAA, particularly rhGAA having a high M6P content. The rhGAA captured by the protein capturing system 605 is eluted from the column(s) by changing the pH and/or salt content in the column. Exemplary conditions for an AEX column are provided in Table 2.

TABLE 2
Exemplary conditions for an AEX column

| | | Flow rate | Volume | Temperature |
|---|---|---|---|---|
| Procedure | Buffer | (cm/h) | (CV) | (° C.) |
| Pre-used | 0.1-10M NaOH | ≤25-2500 | ≥1-3 | 15-25 |
| Sanitization | | | (≥10-120 min) | |
| Pre- | 20-2000 mM phosphate | ≤25-2500 | ≥1-5 | 15-25 |
| Equilibration | buffer (PB), pH 6.9-7.3 | | | |
| Equilibration | 4-400 mM PB, pH 6.9-7.3 | ≤25-2500 | ≥1-5 | 2-15 |
| Load | NA | ≤10-1000 | NA | 2-15 |
| Wash1 | 4-400 mM PB, pH 6.9-7.3 | ≤25-2500 | ≥2-10 | 2-15 |
| Wash2 | 4-400 mM PB, pH 6.9-7.3 | ≤25-2500 | ≥2-10 | 15-25 |
| Elution | 4-400 mM PB, 20-2000 mM | ≤25-2500 | NA | 15-25 |
| | NaCl, pH 6.1-6.5 | | | |
| Strip | 4-400 mM PB, 0.1-10M | ≤25-2500 | ≥1-5 | 15-25 |
| | NaCl, pH 6.1-6.5 | | | |
| Post-use | 0.1-10M NaOH | ≤25-2500 | ≥1-3 | 15-25 |
| Sanitization | | | (≥10-120 min) | |
| Storage | 0.01-1.0M NaOH | ≤25-2500 | ≥1-5 | 15-25 |

The eluted rhGAA can be subjected to further purification steps and/or quality assurance steps. For example, the eluted rhGAA may be subjected to a virus kill step 607. Such a virus kill 607 may include one or more of a low pH kill, a detergent kill, or other technique known in the art. The rhGAA from the virus kill step 607 may be introduced into a second chromatography system 609 to further purify the rhGAA product. Alternatively, the eluted rhGAA from the protein capturing system 605 may be fed directly to the second chromatography system 609. In various embodiments, the second chromatography system 609 includes one or more immobilized metal affinity chromatography (IMAC) columns for further removal of impurities. Exemplary conditions for an IMAC column are provided in Table 3 below.

TABLE 3
Exemplary conditions for an IMAC column

| | | Flow rate | Vol |
|---|---|---|---|
| Procedure | Buffer | (cm/h) | (CV) |
| Rinse | 4-400 mM PB, pH 6.3-6.7 | ≤25-2500 | ≥1-5 |
| Pre-use | 0.01-1.0M NaOH | ≤25-2500 | ≥1-3 |
| Sanitization | | | (10-30 min) |
| Equilibration | 4-400 mM PB, pH 6.5 | ≤25-2500 | ≥1-5 |
| Wash with WFI | Water For Injection (WFI) | ≤25-2500 | ≥1-3 |
| Chelating | 0.01-1.0M Copper Acetate | ≤25-2500 | ≥1-5 |
| Wash with WFI | WFI | ≤25-2500 | ≥2-10 |
| Wash with | 2-200 mM Sodium Acetate, | ≤25-2500 | ≥2-10 |
| acidic buffer | 0.05-5M NaCl, pH 3.5-4.5 | | |
| Equilibration | 4-400 mM PB, pH 6.3-6.7 | ≤25-2500 | ≥1-5 |
| Blank run with | 4-400 mM PB, 15-1500 mM | ≤25-2500 | ≥2-20 |
| elution buffer | Glycine, pH 6.1-6.5 | | |
| Equilibration | 4-400 mM PB, pH 6.3-6.7 | ≤25-2500 | ≥1-5 |
| Load | NA | ≤25-2500 | ≥1-5 |
| Wash1 | 4-400 mM PB, pH 6.3-6.7 | ≤25-2500 | ≥2-10 |
| Wash2 | 4-400 mM PB, 0.1-10M NaCl, | ≤25-2500 | ≥2-10 |
| | 5-30% propylene glycol, | | |
| | pH 6.3-6.7 | | |
| Wash3 | 4-400 mM PB, pH 6.3-6.7 | ≤25-2500 | ≥2-10 |
| Elution | 4-400 mM PB, 15-1500 mM | ≤25-2500 | NA |
| | Glycine, pH 6.1-6.5 | | |
| Strip | 4-400 mM PB, 50-5000 mM | ≤25-2500 | ≥1-5 |
| | imidazole, pH 6.3-6.7 | | |
| Post-use | 0.01-1M NaOH | ≤25-2500 | ≥1-3 |
| Sanitization | | | (10-30 min) |
| Rinse | 4-400 mM PB, pH 6.3-6.7 | ≤25-2500 | ≥1-5 |
| Storage | 5-30% ethanol | ≤25-2500 | ≥1-5 |

After the rhGAA is loaded onto the second chromatography system 609, the recombinant protein is eluted from the column(s). The eluted rhGAA can be subjected to a virus kill step 611. As with virus kill 607, virus kill 611 may include one or more of a low pH kill, a detergent kill, or other technique known in the art. In some embodiments, only one of virus kill 607 or 611 is used, or the virus kills are performed at the same stage in the purification process.

The rhGAA from the virus kill step 611 may be introduced into a third chromatography system 613 to further purify the recombinant protein product. Alternatively, the eluted recombinant protein from the second chromatography system 609 may be fed directly to the third chromatography system 613. In various embodiments, the third chromatography system 613 includes one or more cation exchange chromatography (CEX) columns and/or size exclusion chromatography (SEC) columns for further removal of impurities. The rhGAA product is then eluted from the third chromatography system 613. Exemplary conditions for a CEX column are provided in Table 4 below.

TABLE 4
Exemplary conditions for a CEX column

| | | Flow rate | Vol |
|---|---|---|---|
| Procedure | Buffer | (cm/h) | (CV) |
| Pre-used | 0.1-10M NaOH | ≤25-2500 | ≥1-3 |
| Sanitization | | | (≥10-120 min) |
| Equilibration | 2-200 mM Sodium citrate, pH 4.0-5.0 | ≤30-3000 | ≥2-10 |
| Load | NA | ≤30-3000 | NA |
| Wash | 2-200 mM Sodium citrate, pH 4.0-5.0 | ≤30-3000 | ≥2-10 |
| Elution | 2-200 mM Sodium citrate, 15-1500 mM | ≤30-3000 | ≥2-10 |
| | NaCl, pH 4.0-5.0 | | |
| Strip | 2-200 mM Sodium citrate, 0.1-10M NaCl, | ≤30-3000 | ≥1-5 |
| | pH 4.0-5.0 | | |
| Post-use | 0.1-10M NaOH | ≤25-2500 | ≥1-3 |
| Sanitization | | | (≥10-120 min) |
| Storage | 0.01-1.0M NaOH | ≤30-3000 | ≥1-5 |

The rhGAA product may also be subjected to further processing. For example, another filtration system 615 may be used to remove viruses. In some embodiments, such filtration can utilize filters with pore sizes between 5 and 50 μm. Other product processing can include a product adjustment step 617, in which the recombinant protein product may be sterilized, filtered, concentrated, stored, and/or have additional components for added for the final product formulation.

As used herein, the term “ATB200” refers to a rhGAA with a high content of N-glycans bearing mono-M6P and bis-M6P, which is produced from a GA-ATB200 cell line and purified using methods described herein.

### V. Pharmaceutical Composition

In various embodiments, a pharmaceutical composition comprising the rhGAA described herein, either alone or in combination with other therapeutic agents, and/or a pharmaceutically acceptable carrier, is provided.

In one or more embodiments, a pharmaceutical composition described herein comprises a pharmaceutically acceptable salt.

In some embodiments, the pharmaceutically acceptable salt used herein is a pharmaceutically-acceptable acid addition salt. The pharmaceutically-acceptable acid addition salt may include, but is not limited to, hydrochloric acid, hydrobromic acid, sulfuric acid, sulfamic acid, nitric acid, phosphoric acid, and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

In some embodiments, the pharmaceutically acceptable salt used herein is a pharmaceutically-acceptable base addition salt. The pharmaceutically-acceptable base addition salt may include, but is not limited to, ammonia or the hydroxide, carbonate, or bicarbonate of ammonium or a metal cation such as sodium, potassium, lithium, calcium, magnesium, iron, zinc, copper, manganese, aluminum, and the like. Salts derived from pharmaceutically-acceptable organic nontoxic bases include, but are not limited to, salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like.

In some embodiments, the rhGAA or a pharmaceutically acceptable salt thereof may be formulated as a pharmaceutical composition adapted for intravenous administration. In some embodiments, the pharmaceutical composition is a solution in sterile isotonic aqueous buffer. Where necessary, the composition may also include a solubilizing agent and a local anesthetic to ease pain at the site of the injection. The ingredients of the pharmaceutical composition may be supplied either separately or mixed together in unit dosage form, for example, as a dry lyophilized powder or water free concentrate in a hermetically sealed container such as an ampule or sachet indicating the quantity of active agent. Where the composition is to be administered by infusion, it may be dispensed with an infusion bottle containing sterile pharmaceutical grade water, saline or dextrose/water. In some embodiments, the infusion may occur at a hospital or clinic. In some embodiments, the infusion may occur outside the hospital or clinic setting, for example, at a subject's residence. Where the composition is administered by injection, an ampule of sterile water for injection or saline may be provided so that the ingredients may be mixed prior to administration.

In some embodiments, the rhGAA or a pharmaceutically acceptable salt thereof may be formulated for oral administration. Orally administrable compositions may be formulated in a form of tablets, capsules, ovules, elixirs, solutions or suspensions, gels, syrups, mouth washes, or a dry powder for reconstitution with water or other suitable vehicle before use, optionally with flavoring and coloring agents for immediate-, delayed-, modified-, sustained-, pulsed-, or controlled-release applications. Solid compositions such as tablets, capsules, lozenges, pastilles, pills, boluses, powder, pastes, granules, bullets, dragees, or premix preparations can also be used. Solid and liquid compositions for oral use may be prepared according to methods well known in the art. Such compositions can also contain one or more pharmaceutically acceptable carriers and excipients which can be in solid or liquid form. Tablets or capsules can be prepared by conventional means with pharmaceutically acceptable excipients, including but not limited to binding agents, fillers, lubricants, disintegrants, or wetting agents. Suitable pharmaceutically acceptable excipients are known in the art and include but are not limited to pregelatinized starch, polyvinylpyrrolidone, povidone, hydroxypropyl methylcellulose (HPMC), hydroxypropyl ethylcellulose (HPEC), hydroxypropyl cellulose (HPC), sucrose, gelatin, acacia, lactose, microcrystalline cellulose, calcium hydrogen phosphate, magnesium stearate, stearic acid, glyceryl behenate, talc, silica, corn, potato or tapioca starch, sodium starch glycolate, sodium lauryl sulfate, sodium citrate, calcium carbonate, dibasic calcium phosphate, glycine croscarmellose sodium, and complex silicates. Tablets can be coated by methods well known in the art.

In some embodiments, a pharmaceutical composition described herein may be formulated according to U.S. Pat. No. 10,512,676 and U.S. Provisional Application No. 62/506,574, both incorporated herein by reference in their entirety. For instance, in some embodiments, the pH of a pharmaceutical composition described herein is from about 5.0 to about 7.0 or about 5.0 to about 6.0.

In some embodiments, the pH ranges from about 5.5 to about 6.0. In some embodiments, the pH of the pharmaceutical composition is 6.0. In some embodiments, the pH may be adjusted to a target pH by using pH adjusters (e.g., alkalizing agents and acidifying agents) such as sodium hydroxide and/or hydrochloric acid.

The pharmaceutical composition described herein may comprise a buffer system such as a citrate system, a phosphate system, and a combination thereof. The citrate and/or phosphate may be a sodium citrate or sodium phosphate. Other salts include potassium and ammonium salts. In one or more embodiments, the buffer comprises a citrate. In further embodiments, the buffer comprises sodium citrate (e.g., a mixture of sodium citrate dehydrate and citric acid monohydrate). In one or more embodiments, buffer solutions comprising a citrate may comprise sodium citrate and citric acid. In some embodiments, both a citrate and phosphate buffer are present.

In some embodiments, a pharmaceutical composition described herein comprises at least one excipient. The excipient may function as a tonicity agent, bulking agent, and/or stabilizer. Tonicity agents are components which help to ensure the formulation has an osmotic pressure similar to or the same as human blood. Bulking agents are ingredients which add mass to the formulations (e.g., lyophilized) and provide an adequate structure to the cake. Stabilizers are compounds that can prevent or minimize the aggregate formation at the hydrophobic air-water interfacial surfaces. One excipient may function as a tonicity agent and bulking agent at the same time. For instance, mannitol may function as a tonicity agent and also provide benefits as a bulking agent.

Examples of tonicity agents include sodium chloride, mannitol, sucrose, and trehalose. In some embodiments, the tonicity agent comprises mannitol. In some embodiments, the total amount of tonicity agent(s) ranges in an amount of from about 10 mg/mL to about 50 mg/mL. In further embodiments, the total amount of tonicity agent(s) ranges in an amount of from about 10, 11, 12, 13, 14, or 15 mg/mL to about 16, 20, 25, 30, 35, 40, 45, or 50 mg/mL.

In some embodiments, the excipient comprises a stabilizer. In some embodiments, the stabilizer is a surfactant. In some embodiments, the stabilizer is polysorbate 80. In one or more embodiments, the total amount of stabilizer ranges from about 0.1 mg/mL to about 1.0 mg/mL. In further embodiments, the total amount of stabilizer ranges from about 0.1, 0.2, 0.3, 0.4, or 0.5 mg/mL to about 0.5, 0.6, 0.7, 0.8, 0.9, or 1.0 mg/mL. In yet further embodiments, the total amount of stabilizer is about 0.1, 0.2, 0.3, 0.4, 0.5, 0.6, 0.7, 0.8, 0.9, or 1.0 mg/mL.

In some embodiments, a pharmaceutical composition comprises (a) a rhGAA (such as ATB200), (b) at least one buffer selected from the group consisting of a citrate, a phosphate, and a combination thereof, and (c) at least one excipient selected from the group consisting of mannitol, polysorbate 80, and a combination thereof, and has a pH of (i) from about 5.0 to about 6.0, or (ii) from about 5.0 to about 7.0. In some embodiments, the composition further comprises water. In some embodiments, the composition may further comprise an acidifying agent and/or alkalizing agent.

In some embodiments, the pharmaceutical composition comprises (a) a rhGAA (such as ATB200) at a concentration of about 5-50 mg/mL, about 5-30 mg/mL, or about 15 mg/mL, (b) sodium citrate buffer at a concentration of about 10-100 mM or about 25 mM, (c) mannitol at a concentration of about 10-50 mg/mL, or about 20 mg/mL, (d) polysorbate 80, present at a concentration of about 0.1-1 mg/mL, about 0.2-0.5 mg/mL, or about 0.5 mg/mL, and (e) water, and has a pH of about 6.0. In at least one embodiment, the pharmaceutical composition comprises (a) 15 mg/mL rhGAA (such as ATB200) (b) 25 mM sodium citrate buffer, (c) 20 mg/mL mannitol (d) 0.5 mg/mL polysorbate 80, and (e) water, and has a pH of about 6.0. In some embodiments, the composition may further comprise an acidifying agent and/or alkalizing agent.

In some embodiments, the pharmaceutical composition comprising rhGAA is diluted prior to administration to a subject in need thereof.

In some embodiments, a pharmaceutical composition described herein comprises a chaperone. In some embodiments, the chaperone is miglustat or a pharmaceutically acceptable salt thereof. In another embodiment, the chaperone is duvoglustat or a pharmaceutically acceptable salt thereof.

In some embodiments, a rhGAA described herein is formulated in one pharmaceutical composition while a chaperone such as miglustat is formulated in another pharmaceutical composition. In some embodiments, the pharmaceutical composition comprising miglustat is based on a formulation available commercially as ZAVESCA® (Actelion Pharmaceuticals).

In some embodiments, the pharmaceutical composition described herein may undergo lyophilization (freeze-drying) process to provide a cake or powder. Accordingly, in some embodiments, the pharmaceutical composition described herein pertains to a rhGAA composition after lyophilization. The lyophilized mixture may comprise the rhGAA described herein (e.g., ATB200), buffer selected from the group consisting of a citrate, a phosphate, and combinations thereof, and at least one excipient selected from the group consisting of trehalose, mannitol, polysorbate 80, and a combination thereof. In some embodiments, other ingredients (e.g., other excipients) may be added to the lyophilized mixture. The pharmaceutical composition comprising the lyophilized formulation may be provided vial, which then can be stored, transported, reconstituted and/or administered to a patient.

### VI. Methods of Treatment

### A. Treatment of Diseases

Another aspect of the disclosure pertains to a method of treatment of a disease or disorder related to glycogen storage dysregulation by administering the rhGAA or pharmaceutical composition described herein. In some embodiments, the disease is Pompe disease (also known as acid maltase deficiency (AMD) and glycogen storage disease type II (GSD II)). In some embodiments, the rhGAA is ATB200. In some embodiments, the pharmaceutical composition comprises ATB200. Also provided herein are uses of rhGAA or ATB200 to treat Pompe disease.

In some embodiments, the subject treated by the methods disclosed herein is an ERT-experienced patient. In some embodiments, the subject treated by the methods disclosed herein is an ERT-naive patient.

The rhGAA or pharmaceutical composition described herein is administered by an appropriate route. In one embodiment, the rhGAA or pharmaceutical composition is administered intravenously. In other embodiments, the rhGAA or pharmaceutical composition is administered by direct administration to a target tissue, such as to heart or skeletal muscle (e.g., intramuscular), or nervous system (e.g., direct injection into the brain; intraventricularly; intrathecally). In some embodiments, the rhGAA or pharmaceutical composition is administered orally. More than one route can be used concurrently, if desired.

In some embodiments, the therapeutic effects of the rhGAA or pharmaceutical composition described herein may be assessed based on one or more of the following criteria: (1) cardiac status (e.g., increase of end-diastolic and/or end-systolic volumes, or reduction, amelioration or prevention of the progressive cardiomyopathy that is typically found in GSD-II), (2) pulmonary function (e.g., increase in crying vital capacity over baseline capacity, and/or normalization of oxygen desaturation during crying), (3) neurodevelopment and/or motor skills (e.g., increase in AIMS score), and (4) reduction of glycogen levels in tissue of the individual affected by the disease.

In some embodiments, the cardiac status of a subject is improved by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. The cardiac status of a subject may be assessed by measuring end-diastolic and/or end-systolic volumes and/or by clinically evaluating cardiomyopathy. In some embodiments, the pulmonary function of a subject is improved by 10%, 20%, 30%, 40%, or 50% (or any percentage in-between) after administration of one or more dosages of ATB200 or pharmaceutical composition comprising ATB200, as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, ATB200 or pharmaceutical composition comprising ATB200 improves the pulmonary function of a subject after 1 week, 2 weeks, 3 weeks, 1 month, 2 months, or more from administration (or any time period in between).

In some embodiments, the pulmonary function of a subject is improved by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. The pulmonary function of a subject may be assessed by crying vital capacity over baseline capacity, and/or normalization of oxygen desaturation during crying. In some embodiments, the pulmonary function of a subject is improved by 10%, 20%, 30%, 40%, or 50% (or any percentage in-between) after administration of one or more dosages of ATB200 or pharmaceutical composition comprising ATB200, as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, ATB200 or pharmaceutical composition comprising ATB200 improves the pulmonary function of a subject after 1 week, 2 weeks, 3 weeks, 1 month, 2 months, or more from administration (or any time period in between).

In some embodiments, the neurodevelopment and/or motor skills of a subject is improved by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. The neurodevelopment and/or motor skills of a subject may be assessed by determining an AIMS score. The AIMS is a 12-item anchored scale that is clinician-administered and scored (see Rush J A Jr., Handbook of Psychiatric Measures, American Psychiatric Association, 2000, 166-168). Items 1-10 are rated on a 5-point anchored scale. Items 1-4 assess orofacial movements. Items 5-7 deal with extremity and truncal dyskinesia. Items 8-10 deal with global severity as judged by the examiner, and the patient's awareness of the movements and the distress associated with them. Items 11-12 are yes/no questions concerning problems with teeth and/or dentures (such problems can lead to a mistaken diagnosis of dyskinesia). In some embodiments, the neurodevelopment and/or motor skills of a subject is improved by 10%, 20%, 30%, 40%, or 50% (or any percentage in-between) after administration of one or more dosages of ATB200 or pharmaceutical composition comprising ATB200, as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, ATB200 or pharmaceutical composition comprising ATB200 improves the neurodevelopment and/or motor skills of a subject after 1 week, 2 weeks, 3 weeks, 1 month, 2 months, or more from administration (or any time period in between).

In some embodiments, the glycogen level of a certain tissue of a subject is reduced by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. In some embodiment, the tissue is muscle such as quadriceps, triceps, and gastrocnemius. The glycogen level of a tissue can be analyzed using methods known in the art. The determination of glycogen levels is well known based on amyloglucosidase digestion, and is described in publications such as: Amalfitano et al. (1999), “Systemic correction of the muscle disorder glycogen storage disease type ii after hepatic targeting of a modified adenovirus vector encoding human acid-alphaglucosidase,” Proc Natl Acad Sci USA, 96:8861-8866. In some embodiments, the glycogen level in muscle of a subject is reduced by 10%, 20%, 30%, 40%, or 50% (or any percentage in between) after administration of one or more dosages of ATB200 or pharmaceutical composition comprising ATB200, as compared to that of a subject treated with a vehicle or that of a subject prior to treatment. In certain embodiments, the reduction is achieved after 1 week, 2 weeks, 3 weeks, 1 month, 2 months, or more from administration (or any time period in between). In certain embodiments, ATB200 or pharmaceutical composition comprising ATB200 reduces the glycogen level in muscle of a subject after 1 week, 2 weeks, 3 weeks, 1 month, 2 months, or more from administration (or any time period in between).

### B. Biomarkers

Biomarkers of glycogen accumulation in a subject, such as urine hexose tetrasaccharide (Hex4), may be used to assess and compare the therapeutic effects of enzyme replacement therapy in a subject with Pompe disease. In some embodiments, the therapeutic effect of the rhGAA or a pharmaceutical composition comprising rhGAA on glycogen accumulation is assessed by measuring the levels of urinary Hex4 in a subject.

Biomarkers of muscle injury or damage such as creatine kinase (CK), alanine aminotransferase (ALT), and aspartate aminotransferase (AST) may be used to assess and compare the therapeutic effects of enzyme replacement therapy in a subject with Pompe disease. In some embodiments, the therapeutic effect of the rhGAA or a pharmaceutical composition comprising rhGAA on muscle damage is assessed by measuring the levels of CK, ALT, and/or AST in a subject. In at least one embodiment, the therapeutic effect of the rhGAA or a pharmaceutical composition comprising rhGAA on muscle damage is assessed by measuring the levels of CK in a subject.

Biomarkers such as LAMP-1, LC3, and Dysferlin may also be used to assess and compare the therapeutic effects of the rhGAA or pharmaceutical composition described herein. In Pompe disease, the failure of GAA to hydrolyze lysosomal glycogen leads to the abnormal accumulation of large lysosomes filled with glycogen in some tissues. (Raben et al., JBC 273: 19086-19092, 1998.) Studies in a mouse model of Pompe disease have shown that the enlarged lysosomes in skeletal muscle cannot adequately account for the reduction in mechanical performance, and that the presence of large inclusions containing degraded myofibrils (i.e., autophagic buildup) contributes to the impairment of muscle function. (Raben et al., Human Mol Genet 17: 3897-3908, 2008.) Reports also suggest that impaired autophagy flux is associated with poor therapeutic outcome in Pompe patients. (Nascimbeni et al., Neuropathology and Applied Neurobiology doi: 10.1111/nan.12214, 2015; Fukuda et al., Mol Ther 14: 831-839, 2006.) In addition, late-onset Pompe disease is prevalent in unclassified limb-girdle muscular dystrophies (LGMDs) (Preisler et al., Mol Genet Metab 110: 287-289, 2013), which is a group of genetically heterogeneous neuromuscular diseases with more than 30 genetically defined subtypes of varying severity. IHC examination revealed substantially elevated sarcoplasmic presence of dysferlin in the skeletal muscle fibers of Gaa KO mice.

Various known methods can be used to measure the gene expression level and/or protein level of such biomarkers. For instance, a sample from a subject treated with the rhGAA or pharmaceutical composition described herein can be obtained, such as biopsy of tissues, in particular muscle. In some embodiments, the sample is a biopsy of muscle in a subject. In some embodiments, the muscle is selected from quadriceps, triceps, and gastrocnemius. The sample obtained from a subject may be stained with one or more antibodies or other detection agents that detect such biomarkers or be identified and quantified by mass spectrometry. The samples may also or alternatively be processed for detecting the presence of nucleic acids, such as mRNAs, encoding the biomarkers via, e.g., RT-qPCR methods.

In some embodiments, the gene expression level and/or protein level of one or more biomarkers is measured in a muscle biopsy obtained from an individual prior to and post treatment with the rhGAA or pharmaceutical composition described herein. In some embodiments, the gene expression level and/or protein level of one or more biomarkers is measured in a muscle biopsy obtained from an individual treated with a vehicle. In some embodiments, the gene expression level and/or protein level of one or more biomarkers is reduced by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. In some embodiments, the gene expression level and/or protein level of one or more biomarkers is reduced by 10%, 20%, 30%, 40%, or 50% (or any percentage in-between) after administration of one or more dosages of ATB200 or pharmaceutical composition comprising ATB200, as compared to that of a subject treated with a vehicle or that of a subject prior to treatment. In certain embodiments, the reduction is achieved after 1 week, 2 weeks, 3 weeks, 1 month, 2 months, or more from administration (or any time period in between). In certain embodiments, ATB200 or pharmaceutical composition comprising ATB200 reduces the gene expression level and/or protein level of one or more biomarkers after 1 week, 2 weeks, 3 weeks, 1 month, 2 months, or more from administration (or any time period in between).

### C. Dosages of rhGAA

The pharmaceutical formulation or reconstituted composition is administered in a therapeutically effective amount (e.g., a dosage amount that, when administered at regular intervals, is sufficient to treat the disease, such as by ameliorating symptoms associated with the disease, delaying the onset of the disease, and/or lessening the severity or frequency of symptoms of the disease). The amount which is therapeutically effective in the treatment of the disease may depend on the nature and extent of the disease's effects, and can be determined by standard clinical techniques. In addition, in vitro or in vivo assays may optionally be employed to help identify optimal dosage ranges. In at least one embodiment, a rhGAA described herein or pharmaceutical composition comprising the rhGAA is administered at a dose of about 1 mg/kg to about 100 mg/kg, such as about 5 mg/kg to about 30 mg/kg, typically about 5 mg/kg to about 20 mg/kg. In at least one embodiment, the rhGAA or pharmaceutical composition described herein is administered at a dose of about 5 mg/kg, about 10 mg/kg, about 15 mg/kg, about 20 mg/kg, about 25 mg/kg, about 30 mg/kg, about 35 mg/kg, about 40 mg/kg, about 50 mg/kg, about 50 mg/kg, about 60 mg/kg, about 70 mg/kg, about 80 mg/kg, about 90 mg/kg, or about 100 mg/kg. In some embodiments, the rhGAA is administered at a dose of 5 mg/kg, 10 mg/kg, 20 mg/kg, 50 mg/kg, 75 mg/kg, or 100 mg/kg. In at least one embodiment, the rhGAA or pharmaceutical composition is administered at a dose of about 20 mg/kg. In some embodiments, the rhGAA or pharmaceutical composition is administered concurrently or sequentially with a pharmacological chaperone. In some embodiments, the pharmacological chaperone is miglustat. In at least one embodiment, the miglustat is administered as an oral dose of about 260 mg. In at least one embodiment, the miglustat is administered as an oral dose of about 195 mg. The effective dose for a particular individual can be varied (e.g., increased or decreased) over time, depending on the needs of the individual. For example, in times of physical illness or stress, or if anti-acid α-glucosidase antibodies become present or increase, or if disease symptoms worsen, the amount of rhGAA and/or miglustat can be adjusted.

In some embodiments, the therapeutically effective dose of the rhGAA or pharmaceutical composition described herein is lower than that of conventional rhGAA products. For instance, if the therapeutically effective dose of a conventional rhGAA product is 20 mg/kg, the dose of the rhGAA or pharmaceutical composition described herein required to produce the same as or better therapeutic effects than the conventional rhGAA product may be lower than 20 mg/kg. Therapeutic effects may be assessed based on one or more criteria discussed above (e.g., cardiac status, glycogen level, or biomarker expression). In some embodiments, the therapeutically effective dose of the rhGAA or pharmaceutical composition described herein is at least about 5%, 10%, 15%, 20%, 30%, 40%, 50%, 60%, 70%, 80%, 90%, or more lower than that of conventional rhGAA products.

In some embodiments, the therapeutic effect of the rhGAA or pharmaceutical composition described herein comprises an improvement in motor function, an improvement in muscle strength (upper-body, lower-body, or total-body), an improvement in pulmonary function, decreased fatigue, reduced levels of at least one biomarker of muscle injury, reduced levels of at least one biomarker of glycogen accumulation, or a combination thereof. In some embodiments, the therapeutic effect of the rhGAA or pharmaceutical composition described herein comprises a reversal of lysosomal pathology in a muscle fiber, a faster and/or more effective reduction in glycogen content in a muscle fiber, an increase in six-minute walk test distance, a decrease in timed up and go test time, a decrease in four-stair climb test time, a decrease in ten-meter walk test time, a decrease in gait-stair-gower-chair score, an increase in upper extremity strength, an improvement in shoulder adduction, an improvement in shoulder abduction, an improvement in elbow flexion, an improvement in elbow extension, an improvement in upper body strength, an improvement in lower body strength, an improvement in total body strength, an improvement in upright (sitting) forced vital capacity, an improvement in maximum expiratory pressure, an improvement in maximum inspiratory pressure, a decrease in fatigue severity scale score, a reduction in urine hexose tetrasaccharide levels, a reduction in creatine kinase levels, a reduction in alanine aminotransferase levels, a reduction in asparate aminotransferase levels, or any combination thereof.

In some embodiments, the rhGAA or pharmaceutical composition described herein achieves desired therapeutic effects faster than conventional rhGAA products when administered at the same dose. Therapeutic effects may be assessed based on one or more criteria discussed above (e.g., cardiac status, glycogen level, or biomarker expression). For instance, if a single dose of a conventional rhGAA product decreases glycogen levels in tissue of a treated individual by 10% in a week, the same degree of reduction may be achieved in less than a week when the same dose of the rhGAA or pharmaceutical composition described herein is administered. In some embodiments, when administered at the same dose, the rhGAA or pharmaceutical composition described herein may achieve desired therapeutic effects at least about 1.25, 1.5, 1.75, 2.0, 3.0, or more faster than conventional rhGAA products.

In some embodiments, the therapeutically effective amount of rhGAA (or composition or medicament comprising rhGAA) is administered more than once. In some embodiments, the rhGAA or pharmaceutical composition described herein is administered at regular intervals, depending on the nature and extent of the disease's effects, and on an ongoing basis. Administration at a “regular interval,” as used herein, indicates that the therapeutically effective amount is administered periodically (as distinguished from a one-time dose). The interval can be determined by standard clinical techniques. In certain embodiments, rhGAA is administered bimonthly, monthly, bi-weekly, weekly, twice weekly, or daily. In some embodiments, the rhGAA is administered intravenously twice weekly, weekly, or every other week. The administration interval for a single individual need not be a fixed interval, but can be varied over time, depending on the needs of the individual. For example, in times of physical illness or stress, if anti-rhGAA antibodies become present or increase, or if disease symptoms worsen, the interval between doses can be decreased.

In some embodiments, when used at the same dose, the rhGAA or pharmaceutical composition as described herein may be administered less frequently than conventional rhGAA products and yet capable of producing the same as or better therapeutic effects than conventional rhGAA products. For instance, if a conventional rhGAA product is administered at 20 mg/kg weekly, the rhGAA or pharmaceutical composition as described herein may produce the same as or better therapeutic effects than the conventional rhGAA product when administered at 20 mg/kg, even though the rhGAA or pharmaceutical composition is administered less frequently, e.g., biweekly or monthly. Therapeutic effects may be assessed based on one or more criterion discussed above (e.g., cardiac status, glycogen level, or biomarker expression). In some embodiments, an interval between two doses of the rhGAA or pharmaceutical composition described herein is longer than that of conventional rhGAA products. In some embodiments, the interval between two doses of the rhGAA or pharmaceutical composition is at least about 1.25, 1.5, 1.75, 2.0, 3.0, or more longer than that of conventional rhGAA products.

In some embodiments, under the same treatment condition (e.g., the same dose administered at the same interval), the rhGAA or pharmaceutical composition described herein provides therapeutic effects at a degree superior than that provided by conventional rhGAA products. Therapeutic effects may be assessed based on one or more criteria discussed above (e.g., cardiac status, glycogen level, or biomarker expression). For instance, when compared to a conventional rhGAA product administered at 20 mg/kg weekly, the rhGAA or pharmaceutical composition administered at 20 mg/kg weekly may reduce glycogen levels in tissue of a treated individual at a higher degree. In some embodiments, when administered under the same treatment condition, the rhGAA or pharmaceutical composition described herein provides therapeutic effects that are at least about 1.25, 1.5, 1.75, 2.0, 3.0, or more greater than those of conventional rhGAA products.

### D. Two-Component Therapy

In one or more embodiments, the rhGAA or pharmaceutical composition comprising the rhGAA described herein is administered concurrently or sequentially with a pharmacological chaperone. In some embodiments, the rhGAA or pharmaceutical composition is administered via a different route as compared to the pharmacological chaperone. For instance, a pharmacological chaperone may be administered orally while the rhGAA or pharmaceutical composition is administered intravenously.

In various embodiments, the pharmacological chaperone is miglustat. Without wishing to be bound by any theory, it is believed that when co-administered, miglustat stabilizes ATB200 from denaturation in systemic circulation, which enhances the delivery of the active component ATB200 to lysosomes.

In some embodiments, the miglustat is administered at an oral dose of about 50 mg to about 600 mg. In at least one embodiment, the miglustat is administered at an oral dose of about 200 mg to about 600 mg, or at an oral dose of about 200 mg, about 250 mg, about 300 mg, about 350 mg, about 400 mg, about 450 mg, about 500 mg, about 550 mg, or about 600 mg. In at least one embodiment, the miglustat is administered at an oral dose of about 233 mg to about 500 mg. In at least one embodiment, the miglustat is administered at an oral dose of about 250 to about 270 mg, or at an oral dose of about 250 mg, about 255 mg, about 260 mg, about 265 mg or about 270 mg. In at least one embodiment, the miglustat is administered as an oral dose of about 260 mg.

It will be understood by those skilled in the art that an oral dose of miglustat in the range of about 200 mg to 600 mg or any smaller range therewith can be suitable for an adult patient depending on his/her body weight. For instance, for patients having a significantly lower body weight than about 70 kg, including but not limited to infants, children, or underweight adults, a smaller dose may be considered suitable by a physician. Therefore, in at least one embodiment, the miglustat is administered as an oral dose of from about 50 mg to about 200 mg, or as an oral dose of about 50 mg, about 75 mg, about 100 mg, about 125 mg, about 130 mg, about 150 mg, about 175 mg, about 195 mg, about 200 mg, or about 260 mg. In at least one embodiment, the miglustat is administered as an oral dose of from about 65 mg to about 195 mg, or as an oral dose of about 65 mg, about 130 mg, or about 195 mg.

In some embodiments, the rhGAA is administered intravenously at a dose of about 5 mg/kg to about 20 mg/kg and the miglustat is administered orally at a dose of about 50 mg to about 600 mg. In some embodiments, the rhGAA is administered intravenously at a dose of about 5 mg/kg to about 20 mg/kg and the miglustat is administered orally at a dose of about 50 mg to about 200 mg. In some embodiments, the rhGAA is administered intravenously at a dose of about 5 mg/kg to about 20 mg/kg and the miglustat is administered orally at a dose of about 200 mg to about 600 mg. In some embodiments, the rhGAA is administered intravenously at a dose of about 5 mg/kg to about 20 mg/kg and the miglustat is administered orally at a dose of about 200 mg to about 500 mg. In one embodiment, the rhGAA is administered intravenously at a dose of about 20 mg/kg and the miglustat is administered orally at a dose of about 260 mg. In some embodiments, the rhGAA is administered intravenously at a dose of about 5 mg/kg to about 20 mg/kg and the miglustat is administered orally at a dose of about 130 mg to about 200 mg. In one embodiment, the rhGAA is administered intravenously at a dose of about 20 mg/kg and the miglustat is administered orally at a dose of about 195 mg.

In some embodiments, the miglustat and the rhGAA are administered concurrently. For instance, the miglustat may administered within 10, 9, 8, 7, 6, 5, 4, 3, 2, or 1 minute(s) before or after administration of the rhGAA. In some embodiments, the miglustat is administered within 5, 4, 3, 2, or 1 minute(s) before or after administration of the rhGAA.

In some embodiments, the miglustat and the rhGAA are administered sequentially. In at least one embodiment, the miglustat is administered prior to administration of the rhGAA. In at least one embodiment, the miglustat is administered less than three hours prior to administration of the rhGAA. In at least one embodiment, the miglustat is administered about two hours prior to administration of the rhGAA. For instance, the miglustat may be administered about 1.5 hours, about 1 hour, about 50 minutes, about 30 minutes, or about 20 minutes prior to administration of the rhGAA. In at least one embodiment, the miglustat is administered about one hour prior to administration of the rhGAA.

In some embodiments, the miglustat is administered after administration of the rhGAA. In at least one embodiment, the miglustat is administered within three hours after administration of the rhGAA. In at least one embodiment, the miglustat is administered within two hours after administration of the rhGAA. For instance, the miglustat may be administered within about 1.5 hours, about 1 hour, about 50 minutes, about 30 minutes, or about 20 minutes after administration of the rhGAA.

In some embodiments, the subject fasts for at least two hours before and at least two hours after administration of miglustat.

In some embodiments, the two-component therapy according to this disclosure improves one or more disease symptoms in a subject with Pompe disease compared to (1) baseline, or (2) a control treatment comprising administering alglucosidase alfa and a placebo for the pharmacological chaperone. In such control treatment, a placebo was administered in place of the pharmacological chaperone. In some embodiments, the subject treated by two-component therapy is an ERT-experienced patient. In some embodiments, the subject treated by two-component therapy is an ERT-naive patient.

In some embodiments, the two-component therapy according to this disclosure improves the subject's motor function, as measured by a 6-minute walk test (6MWT). In some embodiments, compared to baseline, the subject's 6-minute walk distance (6MWD) is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks of treatment. In some embodiments, the subject's 6MWD is increased by at least 20 meters or at least 5% after 52 weeks of treatment. In some embodiments, compared to the control treatment, the subject's 6MWD is improved by at least 5, 6, 7, 8, 9, 10, 12, 14, 16, 18, 20, 30, 40, or 50 meters after 12, 26, 38, or 52 weeks of treatment. In some embodiments, compared to the control treatment, the subject's 6MWD is improved by at least 13 meters after 52 weeks of treatment. In some embodiments, the subject has a baseline 6MWD less than 300 meters. In some embodiments, the subject has a baseline 6MWD greater than or equal to 300 meters.

In some embodiments, the two-component therapy according to this disclosure stabilizes the subject's pulmonary function, as measured by a forced vital capacity (FVC) test. In some embodiments, after 12, 26, 38, or 52 weeks of treatment, the subject's percent-predicted FVC is either increased compared to baseline, or decreased by less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% compared to baseline. In some embodiments, after 52 weeks of treatment, the subject's percent-predicted FVC is decreased by less than 1% compared to baseline. In some embodiments, compared to the control treatment, the subject's percent-predicted FVC is significantly improved after treatment. In some embodiments, compared to the control treatment, the subject's percent-predicted FVC is significantly improved by at least 0.5%, 1%, 2%, 3%, 4%, 5%, or 6% after 12, 26, 38, or 52 weeks of treatment. In some embodiments, compared to the control treatment, the subject's percent-predicted FVC is significantly improved by at least 3% after 52 weeks of treatment. In some embodiments, the subject has a baseline FVC less than 55%. In some embodiments, the subject has a baseline FVC greater than or equal to 55%.

In some embodiments, the two-component therapy according to this disclosure improves the subject's motor function, as measured by a gait, stair, gower, chair (GSGC) test. In some embodiments, compared to baseline, the subject's GSGC score is improved as indicated by a decrease of at least 0.1, 0.3, 0.5, 0.7, 1.0, 1.5, or 2.5 points after 12, 26, 38 or 52 weeks of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks of treatment. In some embodiments, compared to the control treatment, the subject's GSGC score is significantly improved as indicated by a decrease of at least 1.0 point after 52 weeks of treatment.

In some embodiments, the two-component therapy according to this disclosure reduces the level of at least one marker of muscle damage after treatment. In some embodiments, the at least one marker of muscle damage comprises creatine kinase (CK). In some embodiments, compared to baseline, the subject's CK level is reduced by at least 10%, 15%, 20%, 25%, 30%, 40%, or 50% after 12, 26, 38, or 52 weeks of treatment. In some embodiments, compared to baseline, the subject's CK level is reduced by at least 20% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks of treatment. In some embodiments, compared to the control treatment, the subject's CK level is significantly reduced by at least 30% after 52 weeks of treatment.

In some embodiments, the two-component therapy according to this disclosure reduces the level of at least one marker of glycogen accumulation after treatment. In some embodiments, the at least one marker of glycogen accumulation comprises urine hexose tetrasaccharide (Hex4). In some embodiments, compared to baseline, the subject's urinary Hex4 level is reduced by at least 10%, 15%, 20%, 25%, 30%, 40%, 50%, or 60% after 12, 26, 38, or 52 weeks of treatment. In some embodiments, compared to baseline, the subject's urinary Hex4 level is reduced by at least 30% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks of treatment. In some embodiments, compared to the control treatment, the subject's urinary Hex4 level is significantly reduced by at least 40% after 52 weeks of treatment.

In some embodiments, the two-component therapy according to this disclosure improves one or more disease symptoms in an ERT-experienced patient subject with Pompe disease compared to (1) baseline, or (2) a control treatment comprising administering alglucosidase alfa and a placebo for the pharmacological chaperone.

In some embodiments, the two-component therapy for an ERT-experienced subject with Pompe disease improves the subject's motor function, as measured by a 6MWT. In some embodiments, compared to baseline, the subject's 6MWD is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks of treatment. In some embodiments, the subject's 6MWD is increased by at least 15 meters or at least 5% after 52 weeks of treatment. In some embodiments, compared to the control treatment, the subject's 6MWD is significantly improved after treatment. In some embodiments, compared to the control treatment, the subject's 6MWD is significantly improved by at least 10, 12, 14, 15, 16, 18, 20, 30, 40, or 50 meters after 12, 26, 38, or 52 weeks of treatment. In some embodiments, compared to the control treatment, the subject's 6MWD is significantly improved by at least 15 meters after 52 weeks of treatment. In some embodiments, the subject has a baseline 6MWD less than 300 meters. In some embodiments, the subject has a baseline 6MWD greater than or equal to 300 meters.

In some embodiments, the two-component therapy for an ERT-experienced subject with Pompe disease improves the subject's pulmonary function, as measured by an FVC test. In some embodiments, after 12, 26, 38, or 52 weeks of treatment, the subject's percent-predicted FVC is increased by at least 0.1%, 0.2%, 0.3%, 0.4%, 0.5%, 1%, 2%, 3%, 4%, or 5% compared to baseline. In some embodiments, after 52 weeks of treatment, the subject's percent-predicted FVC is increased by at least 0.1% compared to baseline. In some embodiments, compared to the control treatment, the subject's percent-predicted FVC is significantly improved after treatment. In some embodiments, compared to the control treatment, the subject's percent-predicted FVC is significantly improved by at least 1%, 2%, 3%, 4%, 5%, 6%, 8%, or 10% after 12, 26, 38, or 52 weeks of treatment. In some embodiments, compared to the control treatment, the subject's percent-predicted FVC is significantly improved by at least 4% after 52 weeks of treatment. In some embodiments, the subject has a baseline FVC less than 55%. In some embodiments, the subject has a baseline FVC greater than or equal to 55%.

In some embodiments, the two-component therapy for an ERT-experienced subject with Pompe disease improves the subject's motor function, as measured by a GSGC test. In some embodiments, compared to baseline, the subject's GSGC score is improved as indicated by a decrease of at least 0.1, 0.3, 0.5, 0.7, 1.0, 1.5, or 2.5 points after 12, 26, 38, or 52 weeks of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks of treatment. In some embodiments, compared to the control treatment, the subject's GSGC score is significantly improved as indicated by a decrease of at least 1.0 point after 52 weeks of treatment.

In some embodiments, the two-component therapy for an ERT-experienced subject with Pompe disease reduces the level of at least one marker of muscle damage after treatment. In some embodiments, the at least one marker of muscle damage comprises CK. In some embodiments, compared to baseline, the subject's CK level is reduced by at least 10%, 15%, 20%, 25%, 30%, 40%, or 50% after 12, 26, 38, or 52 weeks of treatment. In some embodiments, compared to baseline, the subject's CK level is reduced by at least 15% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks of treatment. In some embodiments, compared to the control treatment, the subject's CK level is significantly reduced by at least 30% after 52 weeks of treatment.

In some embodiments, the two-component therapy for an ERT-experienced subject with Pompe disease reduces the level of at least one marker of glycogen accumulation after treatment. In some embodiments, the at least one marker of glycogen accumulation comprises urinary Hex4. In some embodiments, compared to baseline, the subject's urinary Hex4 level is reduced by at least 10%, 15%, 20%, 25%, 30%, 40%, 50%, or 60% after 12, 26, 38, or 52 weeks of treatment. In some embodiments, compared to baseline, the subject's urinary Hex4 level is reduced by at least 25% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks of treatment. In some embodiments, compared to the control treatment, the subject's urinary Hex4 level is significantly reduced by at least 40% after 52 weeks of treatment.

### E. Kit

Another aspect of the disclosure pertains to kits suitable for performing the rhGAA therapy described herein. In one or more embodiments, the kit comprises a container (e.g., vial, tube, bag, etc.) comprising the rhGAA or pharmaceutical composition (either before or after lyophilization) and instructions for reconstitution, dilution and administration. In one or more embodiments, the kit comprises a container (e.g., vial, tube, bag, etc.) comprising a pharmacological chaperone (e.g., miglustat) and a pharmaceutical composition comprising rhGAA (either before or after lyophilization), and instructions for reconstitution, dilution, and administration of rhGAA with the pharmacological chaperone.

### EXAMPLES

### Example 1: Preparation of CHO Cells Producing rhGAA Having a High Content of Mono- or Bis-M6P-Bearing N-Glycans

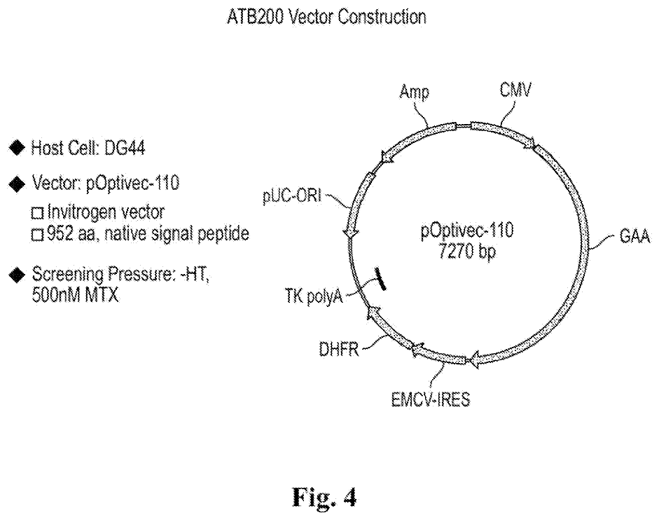

DG44 CHO (DHFR-) cells were transfected with a DNA construct that expresses rhGAA. The DNA construct is shown in FIG. 4. After transfection, CHO cells containing a stably integrated GAA gene were selected with hypoxanthine/thymidine deficient (-HT) medium). GAA expression in these cells was induced by methotrexate treatment (MTX, 500 nM).

Cell pools that expressed high amounts of GAA were identified by GAA enzyme activity assays and were used to establish individual clones producing rhGAA. Individual clones were generated on semisolid media plates, picked by ClonePix system, and were transferred to 24-deep well plates. The individual clones were assayed for GAA enzyme activity to identify clones expressing a high level of GAA. Conditioned media for determining GAA activity used a 4-MU-α-Glucosidase substrate. Clones producing higher levels of GAA as measured by GAA enzyme assays were further evaluated for viability, ability to grow, GAA productivity, N-glycan structure and stable protein expression. CHO cell lines, including CHO cell line GA-ATB200, expressing rhGAA with enhanced mono-M6P or bis-M6P N-glycans were isolated using this procedure.

### Example 2: Purification of rhGAA

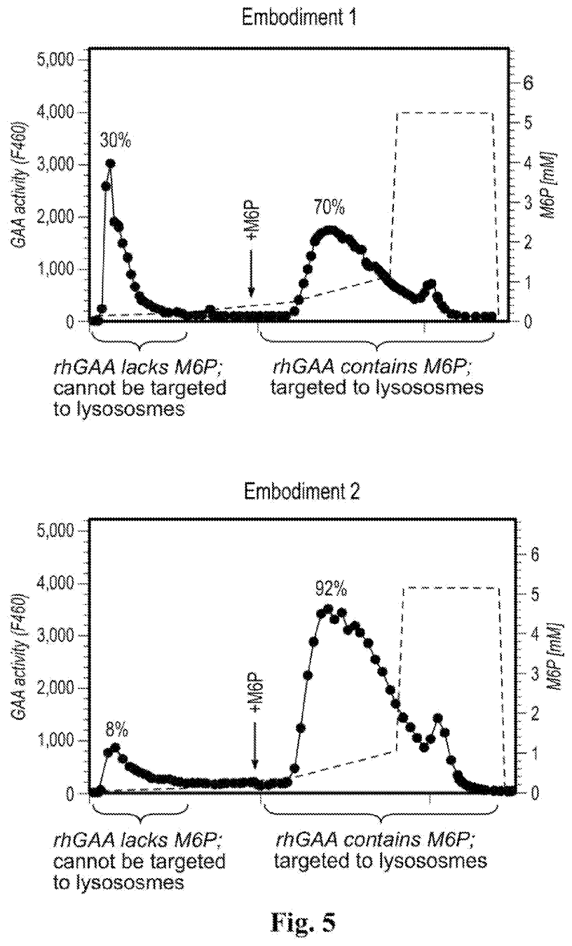

Multiple batches of the rhGAA according to the disclosure were produced in shake flasks and in perfusion bioreactors using CHO cell line GA-ATB200, the product of which is referred to as “ATB200.” Weak anion exchange (“WAX”) liquid chromatography was used to fractionate ATB200 rhGAA according to terminal phosphate and sialic acid. Elution profiles were generated by eluting the ERT with increasing amount of salt. The profiles were monitored by UV (A280 nm). Similar CIMPR receptor binding (at least ˜70%) profiles were observed for purified ATB200 rhGAA from different production batches (FIG. 5), indicating that ATB200 rhGAA can be consistently produced.

### Example 3: Oligosaccharide Characterization of ATB200 rhGAA

ATB200 rhGAA was analyzed for site-specific N-glycan profiles using different LC-MS/MS analytical techniques. The results of the first two LC-MS/MS methods are shown in FIGS. 6A-6H. The results of a third LC-MS/MS method with 2-AA glycan mapping are shown in FIGS. 19A-19H, FIG. 20A-20B, and Table 5.

In the first LC-MS/MS analysis, the protein was denatured, reduced, alkylated, and digested prior to LC-MS/MS analysis. During protein denaturation and reduction, 200 μg of protein sample, 5 μL of 1 mol/L tris-HCl (final concentration 50 mM), 75 μL of 8 mol/L guanidine HCl (final concentration 6 M), 1 μL of 0.5 mol/L EDTA (final concentration 5 mM), 2 μL of 1 mol/L DTT (final concentration 20 mM), and Milli-Q® water were added to a 1.5 mL tube to provide a total volume of 100 μL. The sample was mixed and incubated at 56° C. for 30 minutes in a dry bath. During alkylation, the denatured and reduced protein sample was mixed with 5 μL of 1 mol/L iodoacetamide (IAM, final concentration 50 mM), then incubated at 10-30° C. in the dark for 30 minutes. After alkylation, 400 μL of precooled acetone was added to the sample and the mixture was frozen at −80° C. refrigeration for 4 hours. The sample was then centrifuged for 5 min at 13000 rpm at 4° C. and the supernatant was removed. 400 μL of precooled acetone was added to the pellets, which was then centrifuged for 5 min at 13000 rpm at 4° C. and the supernatant was removed. The sample was then air dried on ice in the dark to remove acetone residue. Forty microliters of 8M urea and 160 μL of 100 mM NH₄HCO₃ were added to the sample to dissolve the protein. During trypsin digestion, 50 μg of the protein was then added with trypsin digestion buffer to a final volume of 100 μL, and 5 μL of 0.5 mg/mL trypsin (protein to enzyme ratio of 20/1 w/w) was added. The solution was mixed well and incubated overnight (16±2 hours) at 37° C. Two and a half microliters of 20% TFA (final concentration 0.5%) were added to quench the reaction. The sample was then analyzed using the Thermo Scientific™ Orbitrap Velos Pro™ Mass Spectrometer.

In the second LC-MS/MS analysis, the ATB200 sample was prepared according to a similar denaturation, reduction, alkylation, and digestion procedure, except that iodoacetic acid (IAA) was used as the alkylation reagent instead of IAM, and then analyzed using the Thermo Scientific™ Orbitrap Fusion™ Lumos Tribid™ Mass Spectrometer.

The results of the first and second analyses are shown in FIGS. 6A-6H. In FIGS. 6A-6H, the results of the first analysis are represented by left bar (dark grey) and the results from the second analysis are represented by the right bar (light grey). The symbol nomenclature for glycan representation is in accordance with Varki, A., Cummings, R. D., Esko J. D., et al., Essentials of Glycobiology, 2nd edition (2009).

As can be seen from FIGS. 6A-6H, the two analyses provided similar results, although there was some variation between the results. This variation can be due to a number of factors, including the instrument used and the completeness of N-glycan analysis. For example, if some species of phosphorylated N-glycans were not identified and/or not quantified, then the total number of phosphorylated N-glycans may be underrepresented, and the percentage of rhGAA bearing the phosphorylated N-glycans at that site may be underrepresented. As another example, if some species of non-phosphorylated N-glycans were not identified and/or not quantified, then the total number of non-phosphorylated N-glycans may be underrepresented, and the percentage of rhGAA bearing the phosphorylated N-glycans at that site may be overrepresented.

FIG. 6A shows the N-glycosylation site occupancy of ATB200. As can be seen from FIG. 6A, the first, second, third, fourth, fifth, and sixth N-glycosylation sites are mostly occupied, with both analyses detecting around or over 90% and up to about 100% of the ATB200 enzyme having an N-glycan detected at each potential N-glycosylation site. However, the seventh potential N-glycosylation site is N-glycosylated about half of the time.

FIG. 6B shows the N-glycosylation profile of the first potential N-glycosylation site, N84. As can be seen from FIG. 6B, the major N-glycan species is bis-M6P N-glycans. Both the first and second analyses detected over 75% of the ATB200 having bis-M6P at the first site, corresponding to an average of about 0.8 mol bis-M6P per mol ATB200 at the first site.

FIG. 6C shows the N-glycosylation profile of the second potential N-glycosylation site, N177. As can be seen from FIG. 6C, the major N-glycan species are mono-M6P N-glycans and non-phosphorylated high mannose N-glycans. Both the first and second analyses detected over 40% of the ATB200 having mono-M6P at the second site, corresponding to an average of about 0.4 to about 0.6 mol mono-M6P per mol ATB200 at the second site.

FIG. 6D shows the N-glycosylation profile of the third potential N-glycosylation site, N334. As can be seen from FIG. 6D, the major N-glycan species are non-phosphorylated high mannose N-glycans, di-, tri-, and tetra-antennary complex N-glycans, and hybrid N-glycans. Both the first and second analyses detected over 20% of the ATB200 having a sialic acid residue at the third site, corresponding to an average of about 0.9 to about 1.2 mol sialic acid per mol ATB200 at the third site.

FIG. 6E shows the N-glycosylation profile of the fourth potential N-glycosylation site, N414. As can be seen from FIG. 6E, the major N-glycan species are bis-M6P and mono-M6P N-glycans. Both the first and second analyses detected over 40% of the ATB200 having bis-M6P at the fourth site, corresponding to an average of about 0.4 to about 0.6 mol bis-M6P per mol ATB200 at the fourth site. Both the first and second analyses also detected over 25% of the ATB200 having mono-M6P at the fourth site, corresponding to an average of about 0.3 to about 0.4 mol mono-M6P per mol ATB200 at the fourth site.

FIG. 6F shows the N-glycosylation profile of the fifth potential N-glycosylation site, N596. As can be seen from FIG. 6F, the major N-glycan species are fucosylated di-antennary complex N-glycans. Both the first and second analyses detected over 70% of the ATB200 having a sialic acid residue at the fifth site, corresponding to an average of about 0.8 to about 0.9 mol sialic acid per mol ATB200 at the fifth site.

FIG. 6G shows the N-glycosylation profile of the sixth potential N-glycosylation site, N826. As can be seen from FIG. 6G, the major N-glycan species are di-, tri-, and tetra-antennary complex N-glycans. Both the first and second analyses detected over 80% of the ATB200 having a sialic acid residue at the sixth site, corresponding to an average of about 1.5 to about 1.8 mol sialic acid per mol ATB200 at the sixth site.

An analysis of the N-glycosylation at the seventh site, N869, showed approximately 40% N-glycosylation, with the most common N-glycans being A4S3S3GF (12%), A5S3G2F (10%), A4S2G2F (8%) and A6S3G3F (8%).

FIG. 6H shows a summary of the phosphorylation at each of the seven potential N-glycosylation sites. A s can be seen from FIG. 6H, both the first and second analyses detected high phosphorylation levels at the first, second, and fourth potential N-glycosylation sites. Both analyses detected over 80% of the ATB200 was mono- or bis-phosphorylated at the first site, over 40% of the ATB200 was mono-phosphorylated at the second site, and over 80% of the ATB200 was mono- or bis-phosphorylated at the fourth site.

Another N-glycosylation analysis of ATB200 was performed according to an LC-MS/MS method as described below. This analysis yielded an average N-glycosylation profile over ten lots of ATB200 (FIGS. 19A-19H, FIGS. 20A-20B).

N-linked glycans from ATB200 were released enzymatically with PNGase-F and labeled with 2-Anthranilic acid (2-AA). The 2-AA labeled N-glycans were further processed by solid phase extraction (SPE) to remove excess salts and other contaminants. The purified 2-AA N-glycans were dissolved in acetonitrile/water (20/80; v/v), and 10 micrograms were loaded on an amino-polymer analytical column (apHera™, Supelco) for High Performance Liquid Chromatography with Fluorescence detection (HPLC-FLD) and High Resolution Mass Spectrometry (HRMS) analysis.

The liquid chromatographic (LC) separation was performed under normal phase conditions in a gradient elution mode with mobile phase A (2% acetic acid in acetonitrile) and mobile phase B (5% acetic acid; 20 millimolar ammonium acetate in water adjusted to pH 4.3 with ammonium hydroxide). The initial mobile phase composition was 70% A/30% B. For the fluorescence detection, the parameters for the detector (RF-20Axs, Shimadzu) were Excitation (Ex):320 nm; Emission (Em):420 nm. The HRMS analysis was carried out using a Quadrupole Time of Flight mass spectrometer (Sciex X500B QTOF) operating in Independent Data Acquisition (IDA) mode. The acquired datafiles were converted into mzML files using MSConvert from ProteoWizard, and then GRITS Toolbox 1.2 Morning Blend software (UGA) was utilized for glycan database searching and subsequent annotation of identified N-glycans. The N-glycans were identified using both precursor monoisotopic masses (m/z) and product ion m/z. Experimental product ions and fragmentation patterns were confirmed in-silico using the GlycoWorkbench 2 Application.

To determine the relative quantitation of N-linked glycans from ATB200, data acquired from the HPLC-FLD-QTOF MS/MS experiment was processed as follows. All of the N-glycan peaks in the FLD chromatogram were integrated, and each peak was assigned a percentage of the total area of all peaks in the FLD chromatogram. The fluorescent signal, expressed as a peak area, is a quantitative measure of the amount of each N-glycan in the sample. However, in most cases, multiple N-glycan species were contained in the same FLD peak. Therefore, the mass spectrometer data was also required to obtain relative quantitation of each N-glycan species (Table 5). The ion intensity signal for each N-glycan was “extracted” from the data to create a chromatographic peak called an extracted ion chromatogram (XIC). The XIC aligned with the FLD chromatographic peak and was specific to only one N-glycan species. The XIC peak created from the ion intensity signal was then integrated and this peak area is a relative quantitative measure of the amount of glycan present. Both the FLD peak areas and mass spectrometer XIC peak areas were used to enable relative quantitation of all the N-linked glycan species of ATB200 reported herein.

The results of this LC-MS/MS analysis are provided in Table 5 below. The symbol nomenclature for glycan representation is in accordance with Wopereis W, et al. 2006. Abnormal glycosylation with hypersialylated O-glycans in patients with Sialuria. Biochimica et Biophysica Acta. 1762:598-607; Gornik O, et al. 2007. Changes of serum glycans during sepsis and acute pancreatitis. Glycobiology. 17:1321-1332; Kattla J J, et al. 2011. Biologic protein glycosylation. In: Murray Moo-Young (ed.), Comprehensive Biotechnology, Second Edition, 3:467-486; Tharmalingam-Jaikaran T, et al. N-glycan profiling of bovine follicular fluid at key dominant follicle developmental stages. 2014. Reproduction. 148:569-580; Clerc F, et al. Human plasma protein N-glycosylation. 2015. Glycoconj J. DOI 10.1007/s10719-015-9626-2; and Blackler R J, et al. 2016. Single-chain antibody-fragment M6P-1 possesses a mannose 6-phosphate monosaccharide-specific binding pocket that distinguishes N-glycan phosphorylation in a branch-specific manner. Glycobiology. 26-2:181-192.

TABLE 5
Type and Prevalence of Oligosaccharides identified on ATB200
based on 2-AA glycan mapping and LC-MS/MS identification

| High Mannose | | Complex | | Complex | | Complex | |
|---|---|---|---|---|---|---|---|
| N-Glycans | % Total | N-Glycans | % Total | N-Glycans | % Total | N-Glycans | % Total |
| 2P-M7 | 11.39 | FA2G2S1 | 3.89 | A3G3S1 + 1Ac | 0.65 | FA2G2S1 + 1Ac | 0.29 |
| P-M7 | 7.97 | FA2G2S2 | 3.42 | A3G2S2 + 1Ac | 0.64 | A4G3 | 0.29 |
| M6 | 6.89 | A2G2S2 | 3.32 | A1G1S1 | 0.63 | A4G4 + 3KDN | 0.29 |
| P-M6 | 3.42 | FA2G2 | 2.77 | A4G3S1 | 0.61 | A4G4S3 | 0.28 |
| M5 | 2.06 | FA4G4S3 | 2.26 | FA3G3 | 0.61 | FA5G4 | 0.24 |
| P-M5 | 1.67 | A2G2S1 | 2.25 | A1G1 | 0.6 | A4G3S2 | 0.21 |
| 2P-M8 | 1.27 | FA3G3S1 | 2.12 | FA2G2S2 + 1Ac | 0.57 | FA1 | 0.21 |
| P-M8 | 1.17 | A3G3S2 | 1.8 | A3G2S1 | 0.57 | FA4G4 | 0.21 |
| BP-M6 | 0.9 | FA2G1 | 1.66 | A3G2S1 | 0.56 | A3G1 | 0.21 |
| M7 | 0.81 | A2G2 | 1.46 | A2G2S2 + 1Ac | 0.5 | FA4G3S2 | 0.21 |
| BP-M7 | 0.69 | FA3G3S1 | 1.42 | FA3G2 | 0.45 | FA3G2S2 | 0.21 |
| M4 | 0.14 | A4G4S1 | 1.28 | A3G3 + 3KDN | 0.45 | A1 | 0.2 |
| BP2-M5 | 0.04 | FA3G3S2 | 1.25 | A4G3S1 | 0.45 | A4G2 | 0.19 |
| BP2-M6 | 0.01 | FA4G4(1LN)S3 | 1.1 | A2G1S1 | 0.41 | FA4G3 | 0.19 |
| Hybrid | % Total | FA4G4S1 | 1.08 | A3G2 | 0.4 | FA3 | 0.18 |
| N-Glycans | | | | | | | |
| FA1P-M6 | 2.16 | A3G3 | 1.08 | FA4G4S1 + LN | 0.4 | A1G1S1 | 0.18 |
| M5A1G1S1 | 1.56 | FA4G4S4 | 1.07 | FA3G2S1 | 0.39 | A4G1S1 | 0.16 |
| FP-M6A1G1S1 | 0.42 | FA3G3S3 | 1.04 | FA2 | 0.38 | FA1G1 | 0.15 |
| A1M5 | 0.36 | FA4G4S2 | 0.94 | FA4G4S2 + LN | 0.38 | FA3G1 | 0.14 |
| A1G1M5 | 0.32 | A2G1 | 0.94 | A3G2S2 | 0.37 | FA5G4S2 | 0.12 |
| P-M6A1G1S1 | 0.17 | FA2G1S1 | 0.94 | A2 | 0.34 | A3G1S1 | 0.11 |
| Summary | Total | A4G4 | 0.91 | FA4G4(2LN)S3 | 0.33 | A3 | 0.11 |
| High Mannose | 38% | FA1G1S1 | 0.91 | FA2G2Sg1 | 0.32 | FA3G3S3 + 1Ac | 0.1 |
| N-Glycans | | | | | | | |
| Hybrid | 5% | FA2G2S2 + 2Ac | 0.76 | FA4G4(1LN)S4 | 0.31 | A2G2S1 + 1Ac | 0.09 |
| N-Glycans | | | | | | | |
| Complex | 57% | A4G4S2 | 0.69 | A3G3S3 | 0.29 | FA3G1S1 | 0.06 |
| N-Glycans | | | | | | | |

Based on this 2-AA and LC-MS/MS analysis, and as further summarized, the ATB200 tested has an average M6P content of 3-5 mol per mol of ATB200 (accounting for both mono-M61P and bis-M6P) and sialic acid content of 4-7 mol per mol of ATB200.

As shown in FIGS. 19A-19H and summarized in FIG. 20B, the first potential N-glycosylation site of ATB200 has an average M6P content of about 1.4 mol M6P/mol ATB200, accounting for an average mono-M61P content of about 0.25 mol mono-M6P/mol ATB200 and an average bis-M61P content of about 0.56 mol bis-M6P/mol ATB200; the second potential N-glycosylation site of ATB200 has an average M6P content of about 0.5 mol M6P/mol ATB200, with the primary phosphorylated N-glycan species being mono-M61P N-glycans; the third potential N-glycosylation site of ATB200 has an average sialic acid content of about 1 mol sialic acid/mol ATB200; the fourth potential N-glycosylation site of ATB200 has an average M6P content of about 1.4 mol M6P/mol ATB200, accounting for an average mono-M6P content of about 0.35 mol mono-M6P/mol ATB200 and an average bis-M6P content of about 0.52 mol bis-M6P/mol ATB200; the fifth potential N-glycosylation site of ATB200 has an average sialic acid content of about 0.86 mol sialic acid/mol ATB200; the sixth potential N-glycosylation site of ATB200 has an average sialic acid content of about 4.2 mol sialic acid/mol ATB200; and the seventh potential N-glycosylation site of ATB200 has an average sialic acid content of about 0.86 mol sialic acid/mol ATB200.

Also according to this 2-AA and LC-MS/MS analytical technique, an average of about 65% of the N-glycans at the first potential N-glycosylation site of ATB200 are high mannose N-glycans, about 89% of the N-glycans at the second potential N-glycosylation site of ATB200 are high mannose N-glycans, over half of the N-glycans at the third potential N-glycosylation site of ATB200 are sialylated (with nearly 20% fully sialylated) and about 85% of the N-glycans at the third potential N-glycosylation site of ATB200 are complex N-glycans, about 84% of the N-glycans at the fourth potential N-glycosylation site of ATB200 are high mannose N-glycans, about 70% of the N-glycans at the fifth potential N-glycosylation site of ATB200 are sialylated (with about 26% fully sialylated) and about 100% of the N-glycans at the fifth potential N-glycosylation site of ATB200 are complex N-glycans, about 85% of the N-glycans at the sixth potential N-glycosylation site of ATB200 are sialylated (with nearly 27% fully sialylated) and about 98% of the N-glycans at the sixth potential N-glycosylation site of ATB200 are complex N-glycans, and about 87% of the N-glycans at the seventh potential N-glycosylation site of ATB200 are sialylated (with nearly 8% fully sialylated) and about 100% of the N-glycans at the seventh potential N-glycosylation site of ATB200 are complex N-glycans.

### Example 4: Analytical Comparison of ATB200 and MYOZYME®/LUMIZYME®

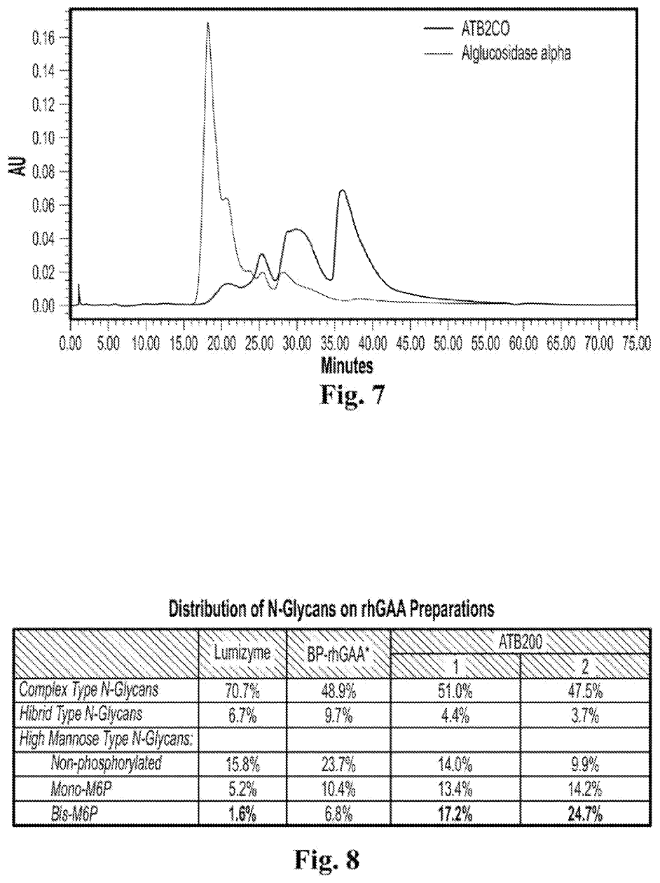

Purified ATB200 and LUMIZYME® N-glycans were evaluated by MALDI-TOF to determine the individual N-glycan structures found on each ERT. LUMIZYME® was obtained from a commercial source. As shown in FIG. 7, ATB200 exhibited four prominent peaks eluting to the right of LUMIZYME®. This confirms that ATB200 was phosphorylated to a greater extent than LUMIZYME® since this evaluation is by terminal charge rather than CIMPR affinity. As summarized in FIG. 8, ATB200 samples were found to contain lower amounts of non-phosphorylated high-mannose type N-glycans than LUMIZYME®.

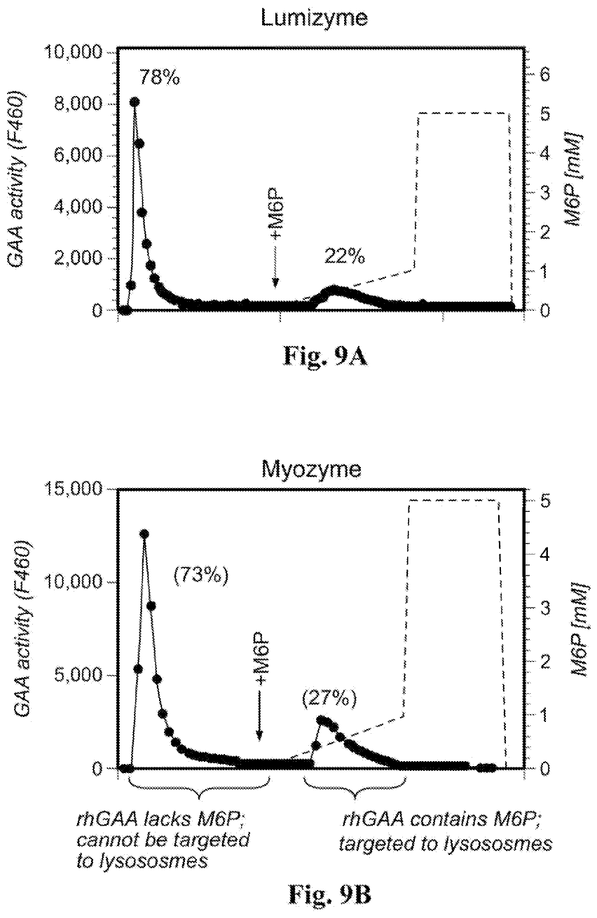

To evaluate the ability of the conventional rhGAAs in MYOZYME® and LUMIZYME® to interact with the CIMPR, the two conventional rhGAA preparations were injected onto a CIMPR affinity column (which binds rhGAA having M6P groups) and the flow through collected. The bound material was eluted with a free M6 gradient. Fractions were collected in 96-well plate and GAA activity assayed by 4MU-α-glucosidase substrate. The relative amounts of unbound (flow through) and bound (M6P eluted) rhGAA were determined based on GAA activity and reported as the fraction of total enzyme. FIGS. 9A and 9B show the binding profile of rhGAAs in MYOZYME® and LUMIZYME®: 73% of the rhGAA in MYOZYME® (FIG. 9B) and 78% of the rhGAA in LUMIZYME® (FIG. 9A) did not bind to the CIMPR. Indeed, only 27% of the rhGAA in MYOZYME® and 22% of the rhGAA in LUMIZYME® contained M6P that can be productive to target it to the CIMPR on muscle cells. In contrast, as shown in FIG. 5, under the same condition, more than 70% of the rhGAA in ATB200 was found to bind to the CIMPR.

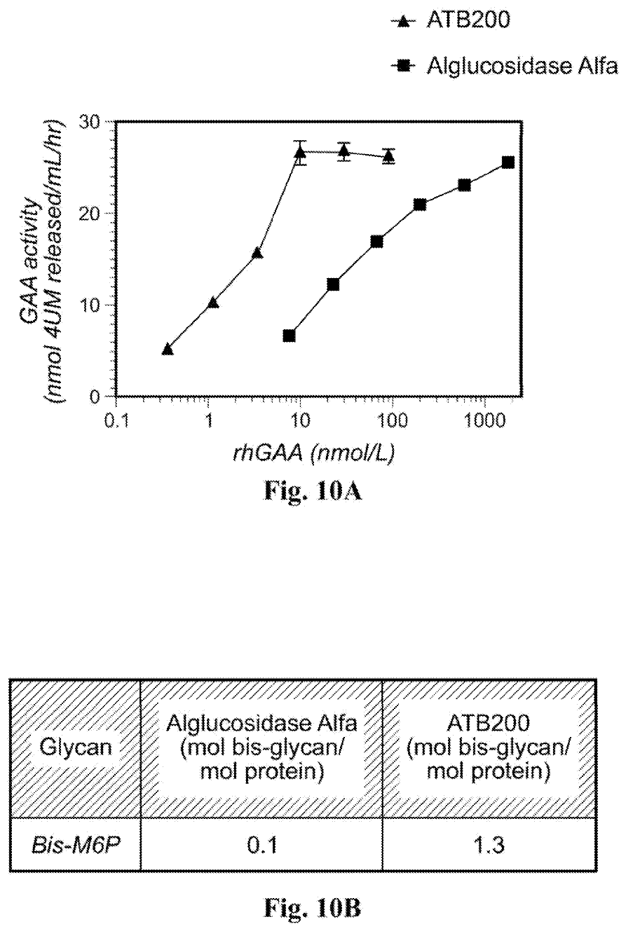

In addition to having a greater percentage of rhGAA that can bind to the CIMPR, it is important to understand the quality of that interaction. LUMIZYME® and ATB200 receptor binding was determined using a CIMPR plate binding assay. Briefly, CIMPR-coated plates were used to capture GAA. Varying concentrations of rhGAA were applied to the immobilized receptor and unbound rhGAA was washed off. The amount of remaining rhGAA was determined by GAA activity. As shown in FIG. 10A, ATB200 bound to CIMPR significantly better than LUMIZYME®. FIG. 10B shows the relative content of bis-M6P N-glycans in LUMIZYME® (a conventional rhGAA product) and ATB200 according to the invention. For LUMIZYME®, there is on average only 10% of molecules having a bis-phosphorylated N-glycan. In contrast, on average every rhGAA molecule in ATB200 has at least one bis-phosphorylated N-glycan.

Overall, the higher content of M6P N-glycans in ATB200 than in LUMIZYME® indicates that the higher portion of rhGAA molecules in ATB200 can target muscle cells. As shown above, the high percentage of mono-phosphorylated and bis-phosphorylated structures determined by MALDI agree with the CIMPR profiles which illustrated significantly greater binding of ATB200 to the CIMPR receptor. N-glycan analysis via MALDI-TOF mass spectrometry confirmed that on average each ATB200 molecule contains at least one natural bis-M6P N-glycan structure. This higher bis-M6P N-glycan content on ATB200 directly correlated with high-affinity binding to CIMPR in M6P receptor plate binding assays (K_{D} about 2-4 nM).

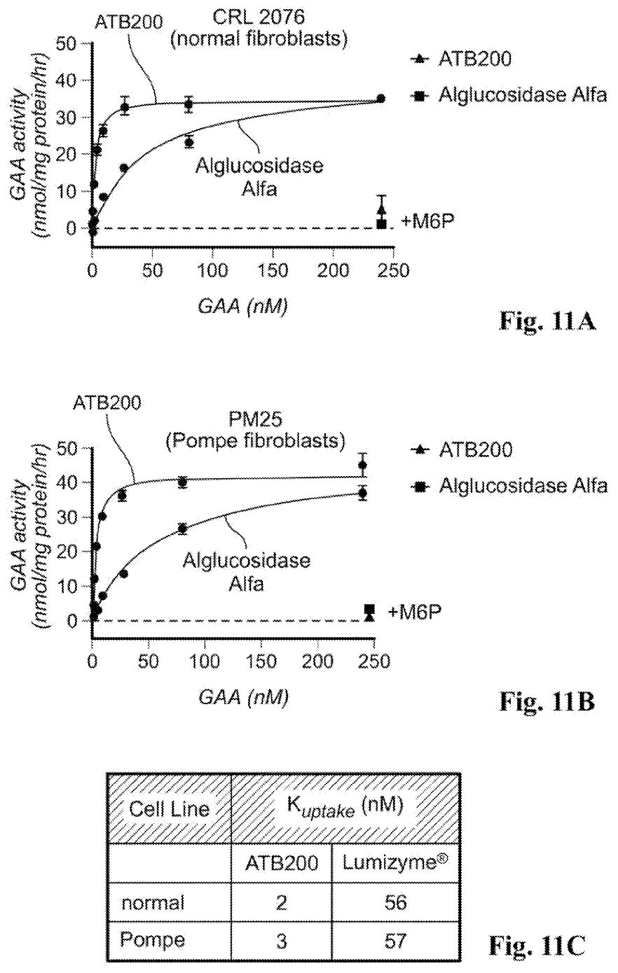

The relative cellular uptake of ATB200 and LUMIZYME® rhGAA were compared using normal and Pompe fibroblast cell lines. Comparisons involved 5-100 nM of ATB200 according to the disclosure with 10-500 nM conventional rhGAA product LUMIZYME®. After 16-hr incubation, external rhGAA was inactivated with TRIS base and cells were washed 3-times with PBS prior to harvest. Internalized GAA measured by 4MU-α-Glucoside hydrolysis and was graphed relative to total cellular protein and the results appear in FIGS. 11A-11C.

ATB200 was also shown to be efficiently internalized into cells. As depicted in FIGS. 11A-11B, ATB200 is internalized into both normal and Pompe fibroblast cells and is internalized to a greater degree than the conventional rhGAA product LUMIZYME®. ATB200 saturates cellular receptors at about 20 nM, while about 250 nM of LUMIZYME®® is needed to saturate cellular receptors. The uptake efficiency constant (Kᵤₚₜₐₖₑ) extrapolated from these results is 2-3 nm for ATB200 and 56 nM for LUMIZYME®, as shown by FIG. 11C. These results suggest that ATB200 is a well-targeted treatment for Pompe disease.

### Example 5: ATB200 and Pharmacological Chaperone

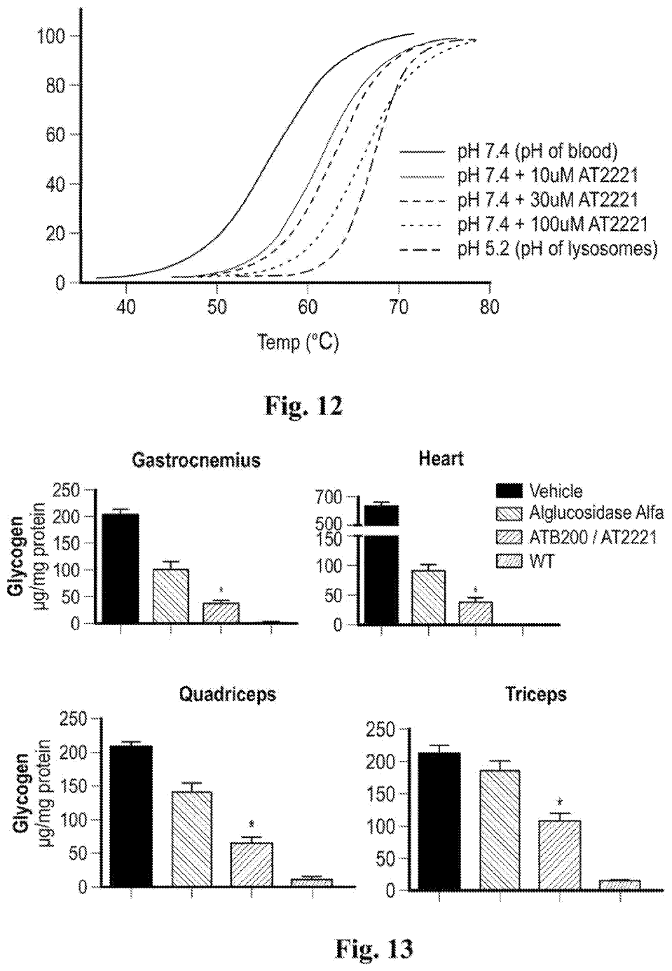

The stability of ATB200 in acidic or neutral pH buffers was evaluated in a thermostability assay using SYPRO Orange, as the fluorescence of the dye increases when proteins denature. As shown in FIG. 12, the addition of AT2221 stabilized ATB200 at pH 7.4 in a concentration-dependent manner, comparable to the stability of ATB200 at pH 5.2, a condition that mimics the acidic environment of the lysosome. As summarized in Table 6, the addition of AT2221 increased the melting temperature (Tm) of ATB200 by nearly 10° C.

TABLE 6
Stability of ATB200 with AT2221

| Test Condition | Tm (° C.) |
|---|---|
| pH 7.4 | 56.2 |
| pH 7.4 + 10 μM AT2221 | 61.6 |
| pH 7.4 + 30 μM AT2221 | 62.9 |
| pH 7.4 + 100 μM AT2221 | 66.0 |
| pH 5.2 | 67.3 |

### Example 6: Co-administration of ATB200 and AT2221 in Gaa KO Mice

The therapeutic effects of ATB200 and AT2221 were evaluated and compared against those of Alglucosidase alfa in Gaa KO mice. For the study, male Gaa KO (3- to 4-month old) and age-matched wild-type (WT) mice were used. Alglucosidase alfa was administered via bolus tail vein intravenous (IV) injection. In the co-administration regimen, AT2221 was administered via oral gavage (PO) 30 minutes prior to the IV injection of ATB200. Treatment was given biweekly. Treated mice were sacrificed after 14 days from the last administration and various tissues were collected for further analysis. Table 7 summarizes the study design:

TABLE 7
Co-administration Study Design

| | | Drug Dosage per Administration | Number of |
|---|---|---|---|
| Genotype | Treatment | (bi-weekly) | Administration |
| Gaa KO | Vehicle | N/A | 6 |
| Gaa KO | Alglucosidase alfa | 20 mg/kg | 6 |
| Gaa KO | ATB200/AT2221 | 20 mg/kg (ATB200) | 6 |
| | | 10 mg/kg (AT2221) | |
| WT (Sve 129) | Not Treated | N/A | N/A |

Tissue glycogen content in tissues samples was determined using amyloglucosidase digestion, as discussed above. As shown in FIG. 13, a combination of 20 mg/kg ATB200 and 10 mg/kg AT2221 significantly decreased the glycogen content in four different tissues (quadriceps, triceps, gastrocnemius, and heart) as compared to the same dosage of alglucosidase alfa.

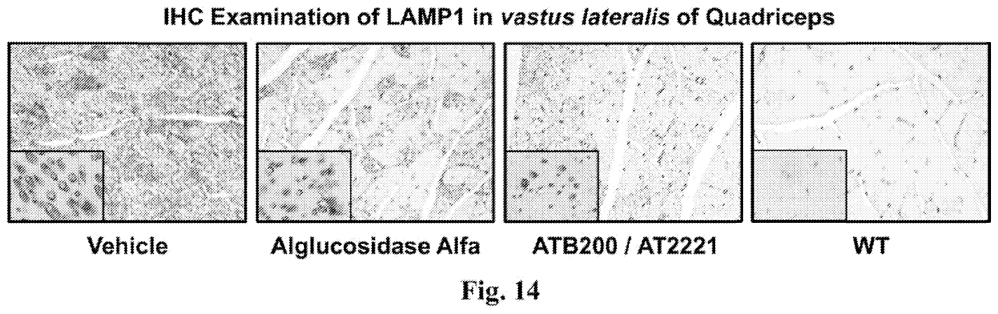

Tissue samples were also analyzed for biomarker changes following the methods discussed in: Khanna R, et al. (2012), “The pharmacological chaperone AT2220 increases recombinant human acid α-glucosidase uptake and glycogen reduction in a mouse model of Pompe disease,” Plos One 7(7): e40776; and Khanna, R et al. (2014), “The Pharmacological Chaperone AT2220 Increases the Specific Activity and Lysosomal Delivery of Mutant Acid α-Glucosidase, and Promotes Glycogen Reduction in a Transgenic Mouse Model of Pompe Disease,” PLoS ONE 9(7): e102092. As shown in FIG. 14, a profound increase in and enlargement of LAMP1-positive vesicles was seen in muscle fibers of Gaa KO animals compared to WT, indicative of lysosomal proliferation. Co-administration of ATB200/AT2221 led to more fibers with normalized LAMP1 level, while the remaining LAMP1-positive vesicles also reduced in size (insets).

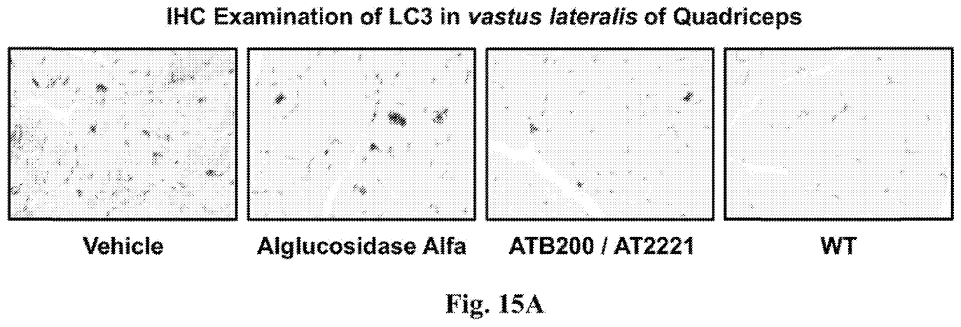

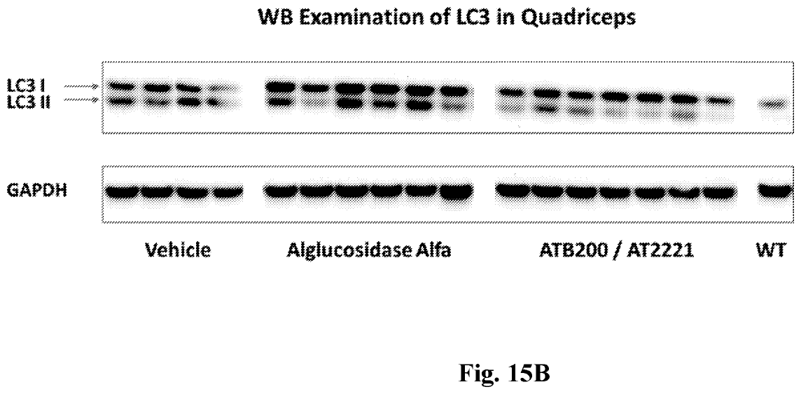

Similarly, intense LC3-positive aggregates in the muscle fibers of untreated Gaa KO mice signify the presence of autophagic zones and autophagy build-up. LC3-positive aggregates (red) were preferentially reduced in mice treated with ATB200/AT2221 co-administration as compared to mice treated with alglucosidase alfa (FIG. 15A). A similar observation was made when the expression of LC3 was assessed using western blot. As shown in FIG. 15B, the majority of animals treated with ATB200/AT2221 showed a significant decrease in levels of LC3 II, the lipidated form that is associated with autophagosomes, suggesting an improved autophagy flux. In comparison, the effect of alglucosidase alfa on autophagy was modest.

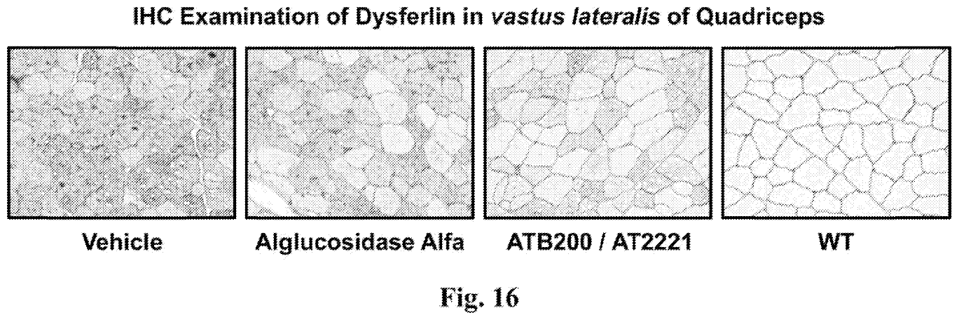

Dysferlin, a protein involved in membrane repair and whose deficiency/mistrafficking is associated with a number of muscular dystrophies, was also assessed. As shown in FIG. 16, dysferlin (brown) was heavily accumulated in the sarcoplasm of Gaa KO mice. Compared to alglucosidase alfa, ATB200/AT2221 was able to restore dysferlin to the sarcolemma in a greater number of muscle fibers.

These data are consistent with improvements at the cellular level demonstrated in human Pompe disease patients treated with ATB200 and miglustat, (e.g., the patients exhibit reduced levels of biomarkers of glycogen accumulation and muscle injury), leading not only to effective treatment of Pompe disease but also a reversal in disease progression. Clinical data in human Pompe disease patients are summarized in Examples 8 and 9, below.

### Example 7: Single Fiber Analysis

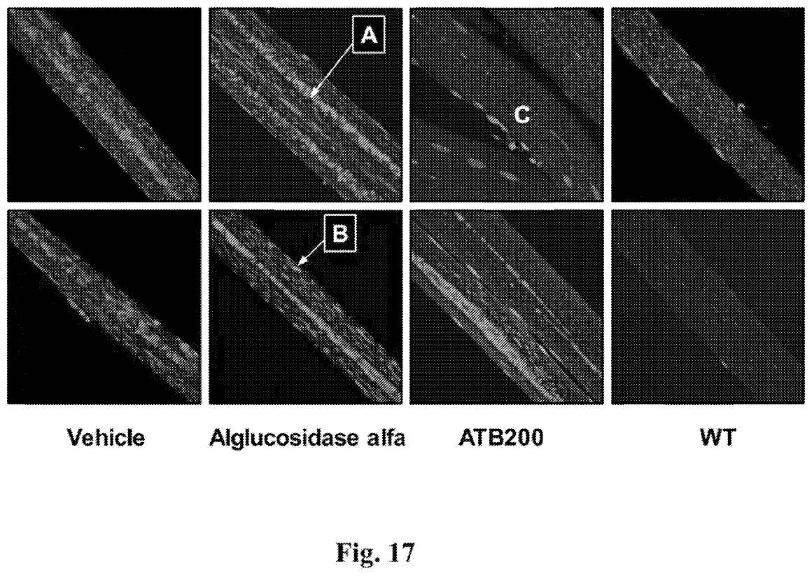

As shown in FIG. 17, majority of the vehicle-treated mice showed grossly enlarged lysosomes (green) (see, for example “B”) and the presence of massive autophagic buildup (red) (see, for example “A”). MYOZYME®-treated mice did not show any significant difference as compared to vehicle-treated mice. In contrast, most fibers isolated from mice treated with ATB200 showed dramatically decreased lysosome size (see, for example, “C”). Furthermore, the area with autophagic buildup was also reduced to various degrees (see, for example, “C”). As a result, a significant portion of muscle fibers analyzed (36-60%) from ATB200-treated mice appeared normal or near-normal. Table 8 below summarizes the single fiber analysis shown in FIG. 17.

TABLE 8
Single Fiber Analysis

| | | | | | Fibers with |
|---|---|---|---|---|---|
| | | Total Number | | Fibers with | Normal or |
| | Animal | of Fibers | Lysosome | Autophagy | Near-normal |
| Treatment | Analyzed | Analyzed (n) | Enlargement | Buildup | Appearance |
| WT | 2 | 65 | − | — | 100% |
| Vehicle | 2 | 65 | + | >90% | <10% |
| Alglucosidase | 4 | 150 | + | >90% | <10% |
| alfa | | | | | |
| ATB200 | 5 | 188 | Dramatic size | 40-64%* | 36-60% |
| | | | decrease in | | |
| | | | most fibers | | |

*This included fibers with varying degree of reduction in autophagic buildup. Overall, the extent of the buildup was smaller in ATB200-treated group compared to Vehicle- or alglucosidase alfa-treated group.

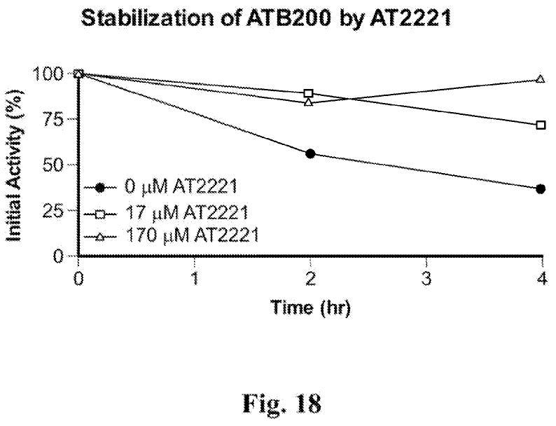

Overall, the data indicate that ATB200, with its higher M6P content, both alone and further stabilized by the pharmacological chaperone AT2221 at the neutral pH of blood, is more efficient in tissue targeting and lysosomal trafficking compared to alglucosidase alfa when administered to Gaa KO mice, consistent with the stabilization of ATB200 by AT2221 as depicted in FIG. 18. As a result, administration of ATB200 and co-administration of ATB200/AT2221 was more effective than alglucosidase alfa in correcting some of the disease-relevant pathologies, such as glycogen accumulation, lysosomal proliferation, and formation of autophagic zones. Due to these positive therapeutic effects, administration of ATB200 and ATB200/AT2221 co-administration is shown to improve the chance of muscle fiber recovery from damage and even to reverse damage by clearing glycogen that had accumulated in the cell due to lack of optimal GAA activity. As with Example 6, these data are also consistent with improvements at the cellular level demonstrated in human Pompe disease patients that lead to both effective treatment of Pompe disease and reversal in disease progression following administration of ATB200 and miglustat. Clinical data in human Pompe disease patients are summarized in Examples 8 and 9, below.

### Example 8: The ATB200-02 Trial

A phase ½ (ATB200-02, NCT-02675465) open-label, fixed-sequence, ascending-dose clinical study was conducted to assess safety, tolerability, pharmacokinetics, pharmacodynamics, and interim efficacy of IV infusion of ATB200 with AT2221 in adult subjects with Pompe disease. The data was reported in International Publication No. WO 2020/163480, the disclosure of which is herein incorporated by reference.

### Example 9: The ATB200-03 Trial: a phase 3 in-human study of ATB200/AT2221 in patients with Pompe disease

The ATB200-03 trial was a phase 3 double-blind, randomized, multicenter, international study of ATB200/AT2221 in adult subjects with late-onset Pompe disease (LOPD) who had received enzyme replacement therapy with alglucosidase alfa (i.e., ERT-experienced) or who had never received ERT (i.e., ERT naïve), compared with alglucosidase alfa/placebo.

### Study Design

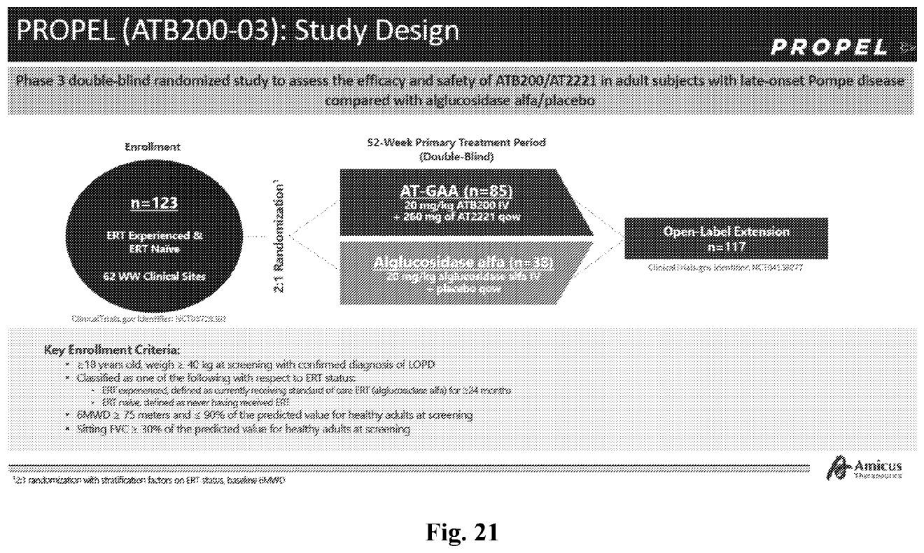

As shown in FIG. 21, the trial consisted of a screening period up to 30 days, a 12-month treatment period, and a 30-day safety follow-up period. Eligible subjects were randomly assigned in a 2:1 ratio to receive ATB200/AT2221 or alglucosidase alfa/placebo and stratified by ERT status (ERT-experienced, ERT-naïve) and baseline 6-minute walk distance (6MWD) (75 to <150 meters, 150 to <400 meters, ≥400 meters).

Efficacy assessments (i.e., functional assessments) included evaluation of ambulatory function (6MWT), motor function tests (Gait, Stair, Gower, and Chair maneuver (GSGC) test and Timed Up and Go (TUG) test), muscle strength (manual muscle testing and quantitative muscle testing), and pulmonary function tests (FVC, SVC, MIP, MEP, and SNIP). Patient reported outcomes (Rasch-built Pompe-specific Activity (R Pact) Scale, EuroQol 5 Dimensions 5 Levels Instrument (EQ-5D-5L), Patient-Reported Outcomes Measurement Information System (PROMIS®) instruments for physical function, fatigue, dyspnea, and upper extremity, and Subject's Global Impression of Change) were recorded. The Physician's Global Impression of Change were also performed.

Pharmacodynamic assessments included measurement of biomarkers of muscle injury (creatine kinase (CK) and disease substrate (urinary hexose tetrasaccharide (Hex4)). Sparse blood samples were collected for determination of total GAA protein levels and AT2221 concentrations in plasma for a population PK analysis in ERT-experienced subjects. Serial blood sampling for characterization of the PK profile of total GAA protein and AT2221 were done in ERT-naïve subjects.

Safety assessments included monitoring of adverse events (AEs), including infusion associated reactions (IARs), clinical laboratory tests (chemistry, hematology, and urinalysis), vital signs, physical examinations including weight, electrocardiograms (ECGs), and immunogenicity. Concomitant medications and nondrug therapies were also be recorded.

### Subject Selection

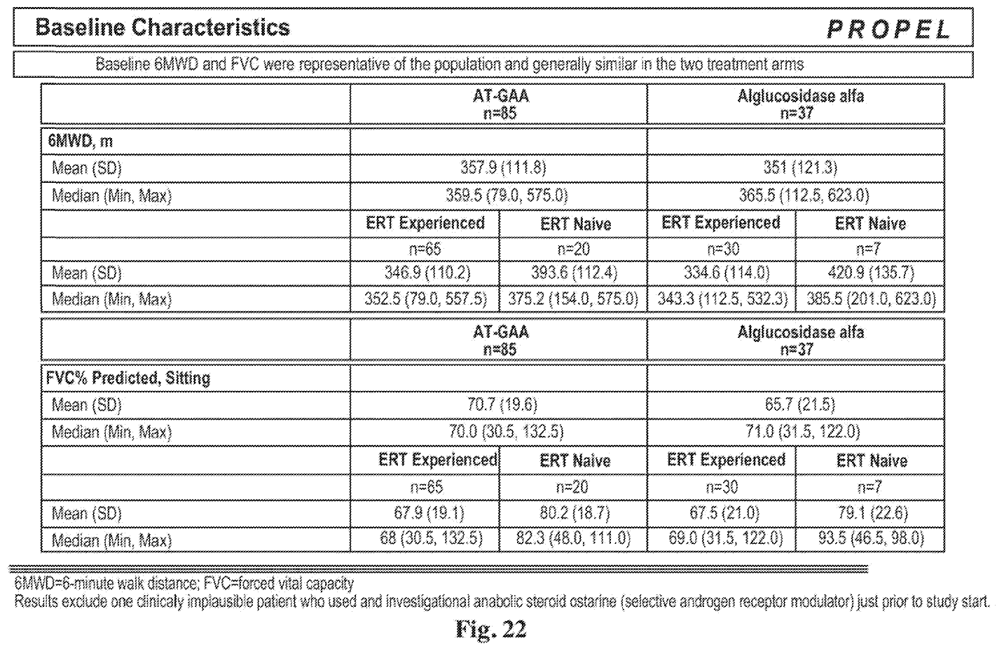

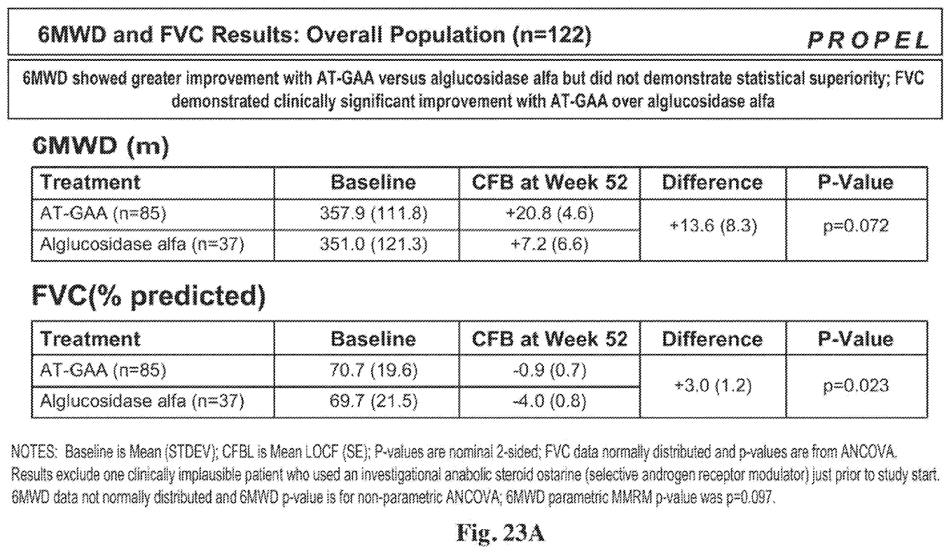

Subjects who participated in the study met all of the following inclusion criteria and none of the exclusion criteria. In total, 122 subjects participated in the ATB200-03 trial. Among them, 85 subjects (ERT-experienced: 65; ERT-naïve: 20) received the ATB200/AT2221 treatment, and 37 subjects (ERT-experienced: 30; ERT-naïve: 7) received the alglucosidase alfa/placebo treatment. As shown in FIG. 22, the baseline 6MWD and FVC data was representative of the population and generally similar in the two treatment arms.

### Inclusion Criteria:

1. Subject provided signed informed consent prior to any study-related procedures being performed.
2. Male and female subjects were ≥18 years old and weighed≥40 kg at screening.
3. Female subjects of childbearing potential and male subjects agreed to use medically accepted methods of contraception during the study and for 90 days after the last dose of study drug.
4. Subject had a diagnosis of LOPD based on documentation of one of the following:
   a. deficiency of GAA enzyme
   b. GAA genotyping
5. Subject was classified as one of the following with respect to ERT status:
   a. ERT-experienced, defined as had received standard of care ERT (alglucosidase alfa) at the recommended dose and regimen (ie, 20 mg/kg dose every 2 weeks) for ≥24 months Specific to Australia, ERT-experienced, defined as had received standard of care ERT (alglucosidase alfa) at the recommended dose and regimen, at a dose of 20 mg/kg based on lean or ideal body weight every 2 weeks
   b. ERT-naïve, defined as never had received investigational or commercially available ERT
6. Subject had a sitting FVC≥30% of the predicted value for healthy adults (National Health and Nutrition Examination Survey III) at screening.
7. Subject performed two 6MWTs at screening that were valid, as determined by the clinical evaluator, and that met all of the following criteria:
   a. both screening values of 6MWD were ≥75 meters
   b. both screening values of 6MWD were ≥90% of the predicted value for healthy adults
   c. the lower value of 6MWD was within 20% of the higher value of 6MWD

### Exclusion Criteria:

1. Subject had received any investigational therapy or pharmacological treatment for Pompe disease, other than alglucosidase alfa, within 30 days or 5 half-lives of the therapy or treatment, whichever was longer, before Day 1 or was anticipated to do so during the study.
2. Subject had received gene therapy for Pompe disease.
3. Subject was taking any of the following prohibited medications within 30 days before Day 1:
   miglitol
   miglustat
   acarbose
   voglibose
4. Subject required the use of invasive or noninvasive ventilation support for >6 hours per day while awake.
5. Subject had a hypersensitivity to any of the excipients in ATB200, alglucosidase alfa, or AT2221.
6. Subject had a medical condition or any other extenuating circumstance that, in the opinion of the investigator or medical monitor, posed an undue safety risk to the subject or compromised his/her ability to comply with or adversely impact protocol requirements. This included clinical depression (as diagnosed by a psychiatrist or other mental health professional) with uncontrolled or poorly controlled symptoms.
7. Subject, if female, was pregnant or breastfeeding at screening.
8. Subject, whether male or female, was planning to conceive a child during the study.
9. Subject refused to undergo genetic testing.

### Investigational Product, Dosage, and Mode of Administration

Subjects were randomized with a randomization ratio of at least 2:1 to receive either ATB200/AT2221 or alglucosidase alfa/placebo. Table 9 below summarizes the treatment of the enrolled subjects.

TABLE 9
Treatment Assignment and Regimen

| Treatment | | |
|---|---|---|
| Assignment | Treatment | Regimen |
| ATB200/ | AT2221^{a} | Subjects ≥50 kg: 260 mg (4 × 65-mg oral |
| AT2221 | | capsules) 1 hour prior to ATB200 infusion |
| | | every 2 weeks |
| | | Subjects ≥40 kg to <50 kg: 195 mg |
| | | (3 × 65-mg oral capsules) 1 hour prior to |
| | | ATB200 infusion every 2 weeks |
| | ATB200 | 20 mg/kg IV infusion over a 4-hour |
| | | duration every 2 weeks |
| Alglucosidase | Placebo | Subjects ≥50 kg: Placebo (4 oral capsules) |
| alfa/placebo | | 1 hour prior to alglucosidase alfa infusion |
| | | every 2 weeks |
| | | Subjects ≥40 kg to <50 kg: Placebo (3 oral |
| | | capsules) 1 hour prior to alglucosidase alfa |
| | | infusion every 2 weeks |
| | Alglucosidase | 20 mg/kg IV infusion over a 4-hour |
| | alfa | duration every 2 weeks |

Abbreviations: IV = intravenous
^{a}Note:
Subjects were required to fast at least 2 hours before and 2 hours after administration of AT2221 or placebo.

### Data Evaluation and Statistical Considerations

The primary efficacy endpoint was the change from baseline to Week 52 in 6MWD. The primary endpoint was tested for superiority of ATB200/AT2221 vs Alglucosidase alfa/placebo, using mixed-effect model for repeated measures (MMRM) and pre-specified nonparametric test in case of violation of normality.

Key secondary efficacy endpoints in a pre-specified hierarchical order of importance were as follows. These secondary endpoints were analyzed using analysis of covariance (ANCOVA) model with last observation carried forward (ITT LOCF).
change from baseline to Week 52 in sitting FVC (% predicted)
change from baseline to Week 52 in the manual muscle test score for the lower extremities
change from baseline to Week 52 in the total score for the PROMIS—physical function
change from baseline to Week 52 in the total score for the PROMIS—fatigue
change from baseline to Week 52 in GSGC total score

Other secondary efficacy endpoints were as follows:
change from baseline to Week 52 in the following variables related to motor function:
   time to complete the 10-meter walk (ie, assessment of gait) of the GSGC test
   time to complete the 4-stair climb of the GSGC test
   time to complete the Gower's maneuver of the GSGC test
   time to arise from a chair as part of the GSGC test
   time to complete the TUG test
change from baseline to Week 52 in the following variables related to muscle strength:
   manual muscle test score for the upper extremities
   manual muscle test total score
   quantitative muscle test value (kg) for the upper extremities
   quantitative muscle test value (kg) for the lower extremities
   quantitative muscle test total value (kg)
change from baseline to Week 52 in the following variables from patient-reported outcome measures:
   total score for the PROMIS—dyspnea
   total score for the PROMIS—upper extremity
   R-PAct Scale total score
   EQ-5D-5L health status
actual value of the subject's functional status (improving, stable, or declining) pertaining to the effects of study drug in the following areas of life at Week 52, as measured by the Subject's Global Impression of Change
   overall physical wellbeing
   effort of breathing
   muscle strength
   muscle function
   ability to move around
   activities of daily living
   energy level
   level of muscular pain
actual value of the subject's functional status (improving, stable, or declining) at Week 52, as measured by the Physician's Global Impression of Change
change from baseline to Week 52 in the following measures of pulmonary function, as follows:
   sitting FVC (% predicted)
   MIP (cmH₂O)
   MIP (% predicted)
   MEP (cmH₂O)
   MEP (% predicted)
   SNIP (cmH₂O)

Pharmacodynamic endpoints were as follows:
change from baseline to Week 52 in serum CK level
change from baseline to Week 52 in urinary Hex4 level

For ERT-experienced subjects, pharmacokinetic endpoints from a population PK analysis of total GAA protein level and AT2221 concentration were collected. For ERT-naïve subjects, PK parameters for plasma total GAA protein concentration and AT2221 were calculated.

The safety profile of ATB200/AT2221 was characterized using incidence of treatment emergent adverse events (TEAEs), serious adverse events (SAEs), and AEs leading to discontinuation of study drug, frequency and severity of immediate and late IARs, and any abnormalities noted in other safety assessments. The impact of immunogenicity to ATB200 and alglucosidase alfa on safety and efficacy was also assessed.

Statistical methods included the following considerations on sample randomization, sample size calculation, efficacy analyses, and safety analyses.

Randomization. The following two factors were identified as design stratification variables: 1. baseline 6MWD (75 to <150 meters, 150 to <400 meters, ≥400 meters); and 2. ERT status (ERT-experienced, ERT-naïve). These two factors formed six factorial combinations (i.e., levels, strata). A centralized block randomization procedure was used to balance the above risk factors, 1) to reduce bias and increase the precision of statistical inference, and 2) to allow various planned and unplanned subset analyses. The block randomization scheme was performed for each of the 6 strata. The randomization ratio is 2:1 ATB200/AT2221 to alglucosidase alfa/placebo, fixed.

Sample Size Calculation. A 2-group t-test with a 2-sided significance level of 0.05 and a 2:1 randomization scheme (66 subjects in the ATB200/AT2221 group and 33 subjects in the alglucosidase alfa/placebo group, for a total sample size of 99 subjects) was determined to have approximately 90% power to detect a standardized effect size of 0.7 between the 2 groups in a superiority test. This calculation was performed using Nquery 8©®. Assuming a 10% dropout rate, the sample size would be approximately 110 subjects.

Efficacy Analyses. The primary efficacy endpoint (i.e., change from baseline to Week 52 in 6MWD) was analyzed using a parametric analysis of covariance (ANCOVA) model to compare between the new treatment and the control. This model would typically adjust for baseline 6MWD (as a continuous covariate), and the 2 factors used to stratify randomization: ERT status (ERT naïve vs. ERT-experienced) and baseline 6MWD (75 to <150 meters, 150 to <400 meters, ≥400 meters). However, the baseline 6MWD could not be used in the model twice (both as a continuous and a categorical variable) due to the expected high point biserial correlation between them. Thus, the 6MWD continuous variable remained in the model but the categorical 6MWD was removed. The ANCOVA model then had terms for treatment, baseline 6MWD (continuous), and ERT status (categorical).

Additionally, potential treatment-by-covariate interactions (i.e., treatment by ERT status and treatment-by-baseline 6MWD continuous) were examined. If an interaction term was statistically significant (e.g., p<0.10, 2 sided), and there was logical biological interpretation, then the interaction term could potentially be added in the final ANCOVA model that would be used for the primary endpoint analysis. The data was then be analyzed based on the ANCOVA model, and all the relevant estimates (e.g., LS means for each treatment group, LS means difference, 95% confidence intervals (CIs) for the LS mean difference, and p-value for comparing between the 2 treatment groups) were provided.

To support the interpretation of clinical benefit, a composite subject-level response was defined based on the totality of the treatment outcome data. Subjects were classified by an ordinal response variable consisting of significant improvement, moderate improvement, or minor/no improvement based on treatment outcomes.

Key secondary endpoints were analyzed according to the hierarchical order, using stepwise closed testing procedure to control the type I error rate. Key secondary and other secondary endpoints were analyzed separately with a similar method used for the primary endpoint analysis.

Safety Analyses. Safety data was summarized using counts and percentages for categorical data and descriptive statistics (mean, standard deviation, median, minimum, maximum) for continuous data.

Efficacy Results from the ATB200-03 Trial

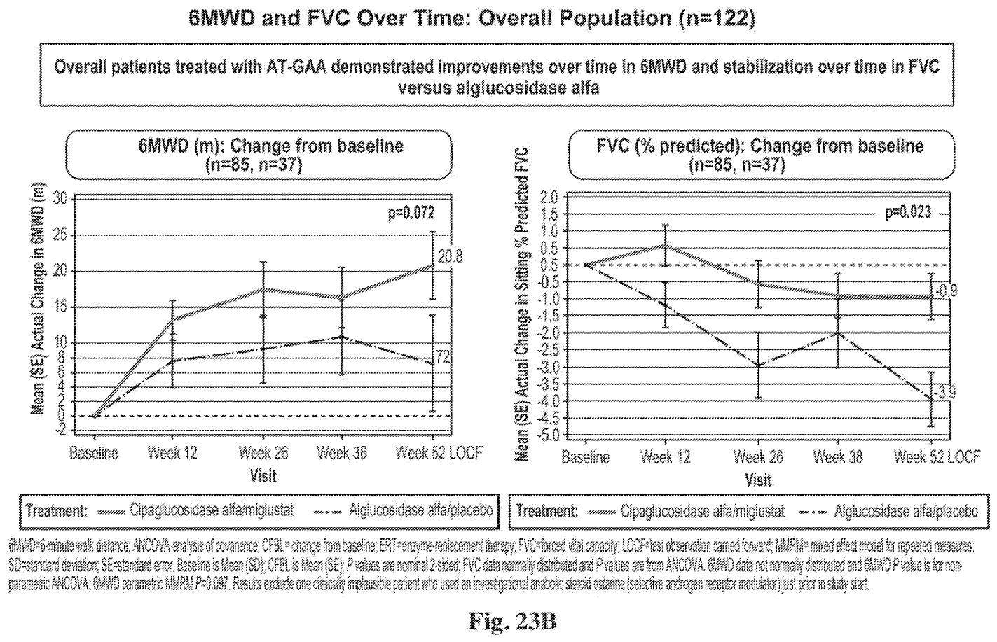

In the overall population, ATB200/AT2221 treatment showed improvement in 6MWD and stabilization in percent-predicted FVC, relative to baseline at week 52 (FIG. 23A) and over time (FIG. 23B). Compared to alglucosidase alfa/placebo, ATB200/AT2221 treatment showed greater improvement in 6MWD in the overall population at week 52 (FIG. 23A). Furthermore, as shown in FIG. 23A, ATB200/AT2221 treatment showed clinically significant improvement in percent-predicted FVC in the overall population at week 52, compared to alglucosidase alfa/placebo.

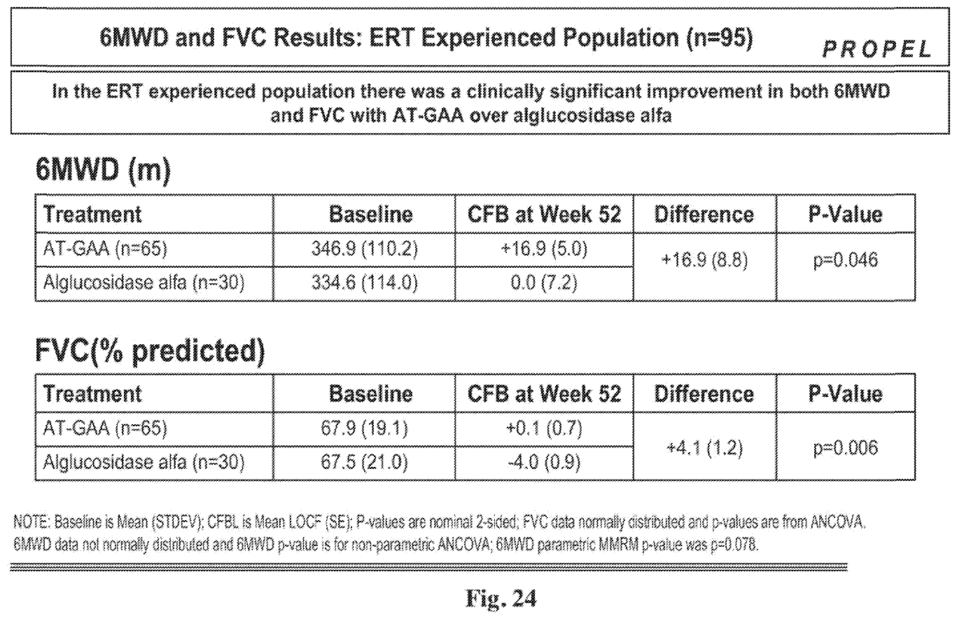

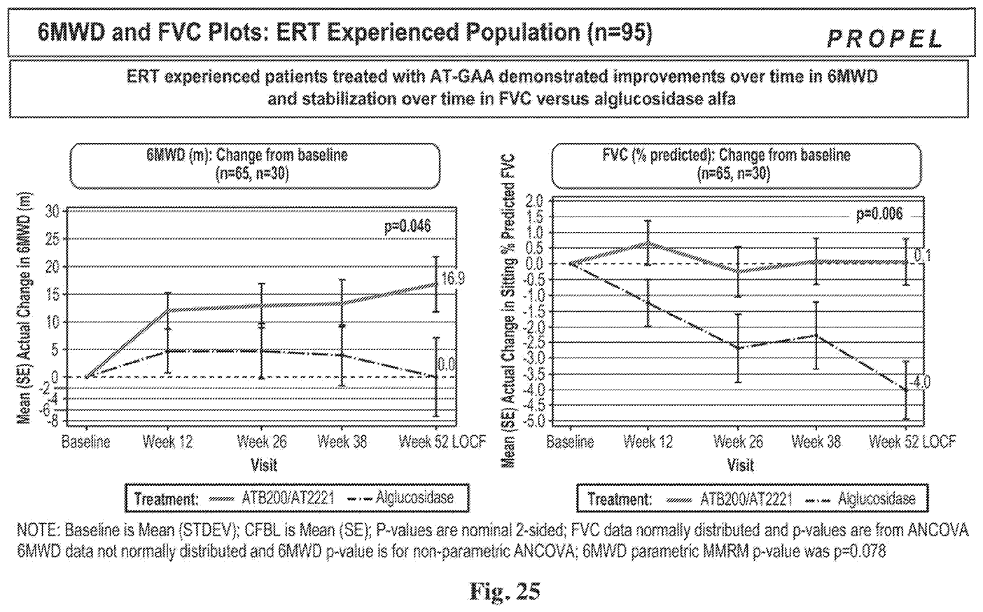

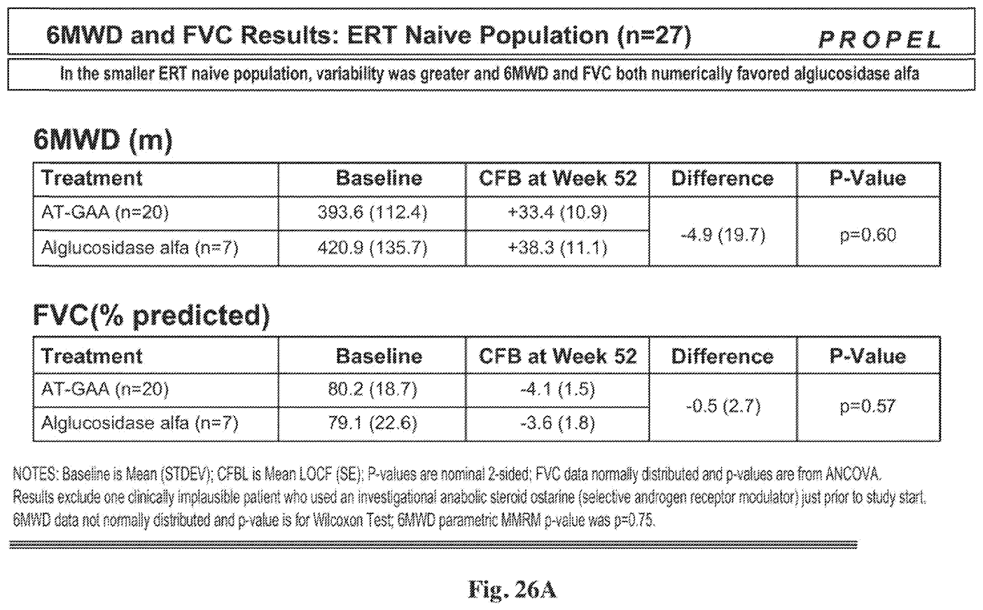

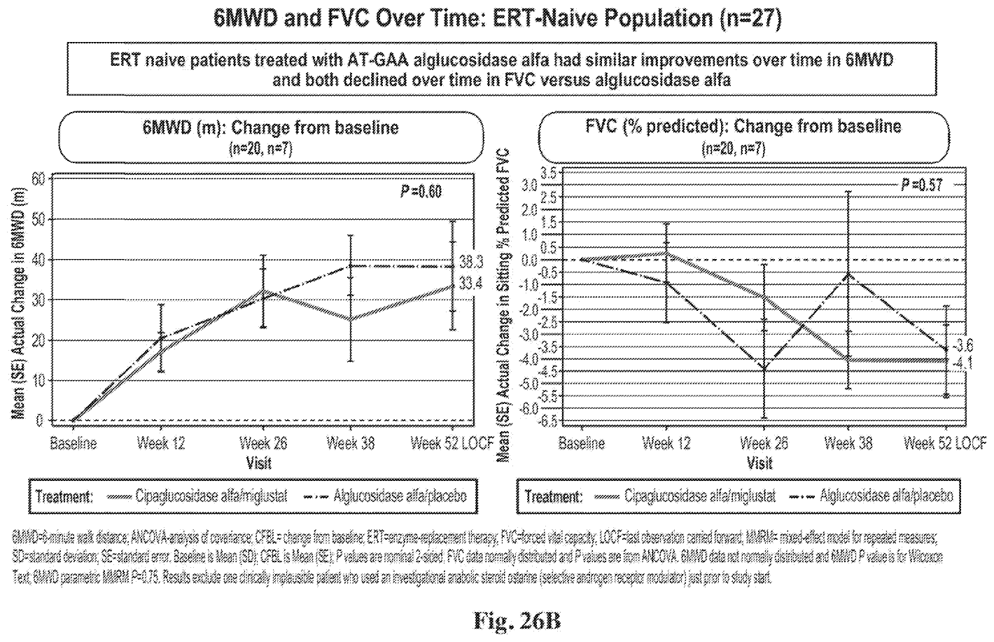

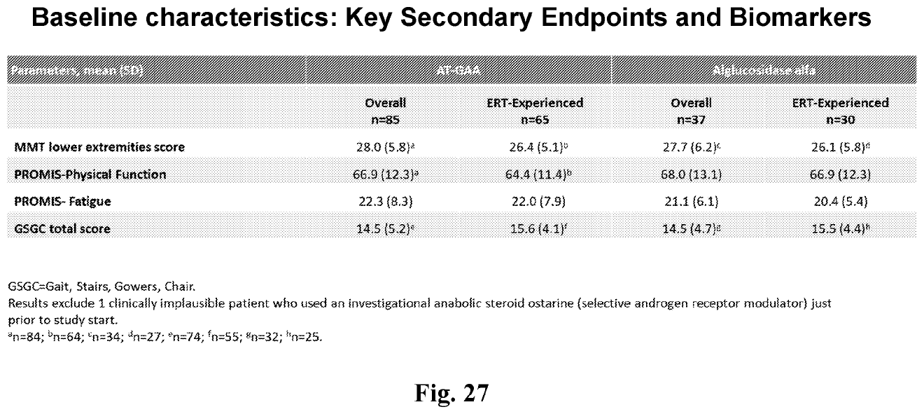

In the ERT-experienced population, ATB200/AT2221 treatment showed improvement in 6MWD and stabilization in percent-predicted FVC, relative to baseline at week 52 (FIG. 24). Compared to alglucosidase alfa/placebo, ATB200/AT2221 treatment showed improvements over time in 6MWD and stabilization over time in percent-predicted FVC in the ERT-experienced population (FIG. 25). Furthermore, as shown in FIG. 24, ATB200/AT2221 treatment showed clinically significant improvement in both 6MWD and percent-predicted FVC in the ERT-experienced population at week 52, compared to alglucosidase alfa/placebo.

As shown in FIGS. 26A and 26B, in the smaller ERT-naive population (n=27), ATB200/AT2221 treatment showed improvement in 6MWD and stabilization in percent-predicted FVC, relative to baseline at week 52 (FIG. 26A) and over time (FIG. 26B). Variability between the two treatment groups was greater and no clinically significant improvement was observed in 6MWD or percent-predicted FVC (FIG. 26A).

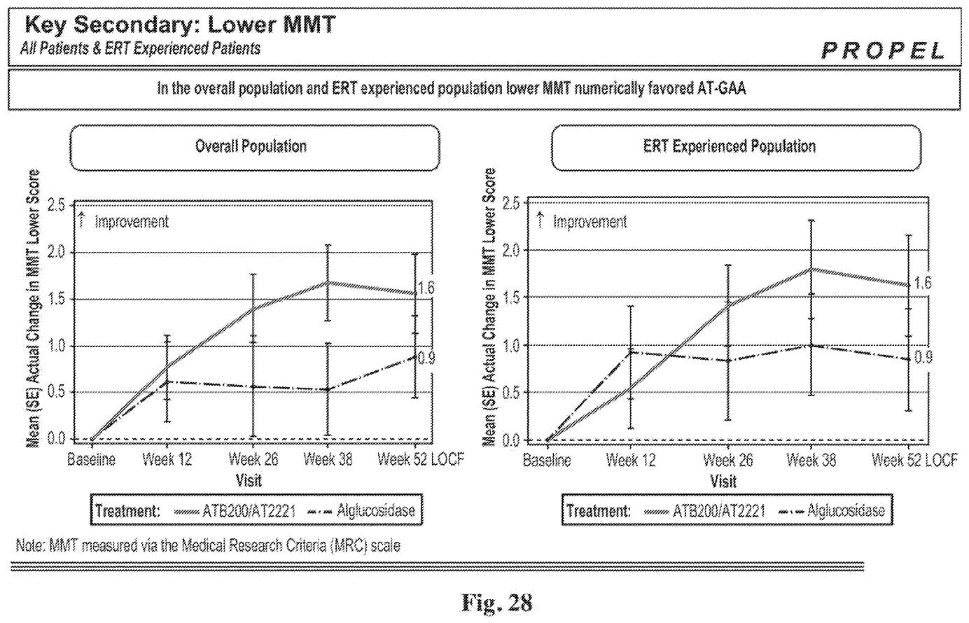

As shown in FIG. 28, in the overall population ERT-experienced populations, lower MMT numerically favored ATB200/AT2221 treatment, compared to alglucosidase alfa/placebo.

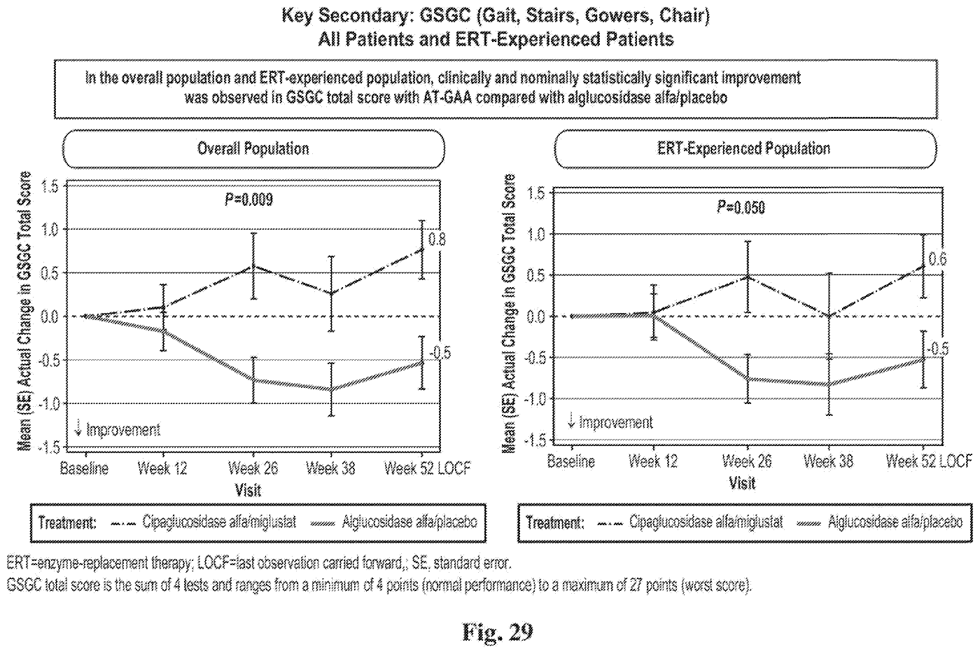

As shown in FIG. 29, in the overall and ERT-experienced populations, ATB200/AT2221 treatment showed clinically significant improvement in GSGC at week 52, compared to alglucosidase alfa/placebo.

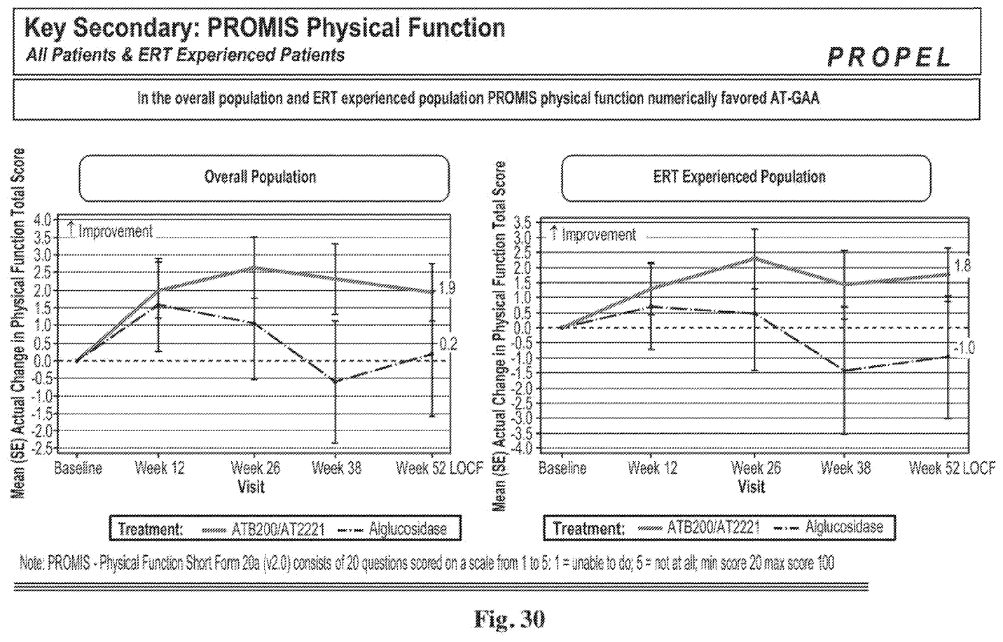

As shown in FIG. 30, in the overall and ERT-experienced populations, PROMIS physical function numerically favored ATB200/AT2221 treatment, compared to alglucosidase alfa/placebo.

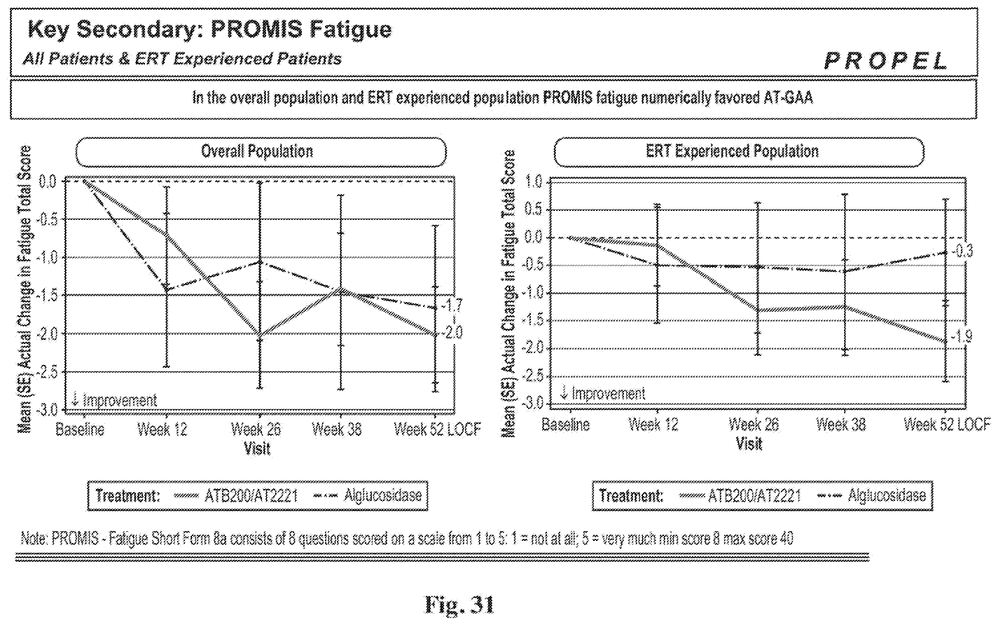

As shown in FIG. 31, in the overall and ERT experienced populations, PROMIS fatigue improved similarly between the two treatment groups.

Biomarker Results from the ATB200-03 Trial

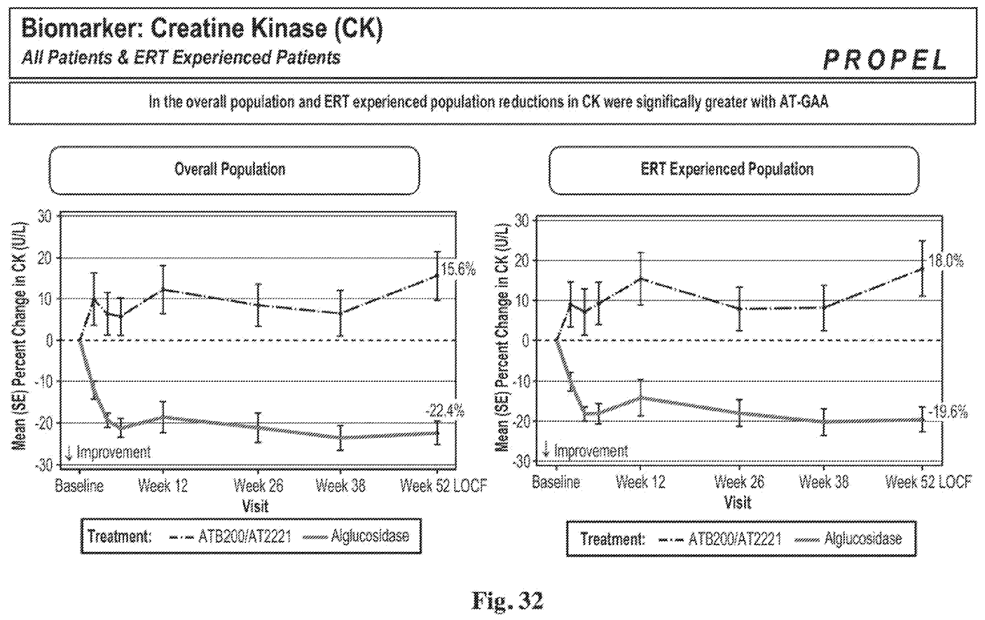

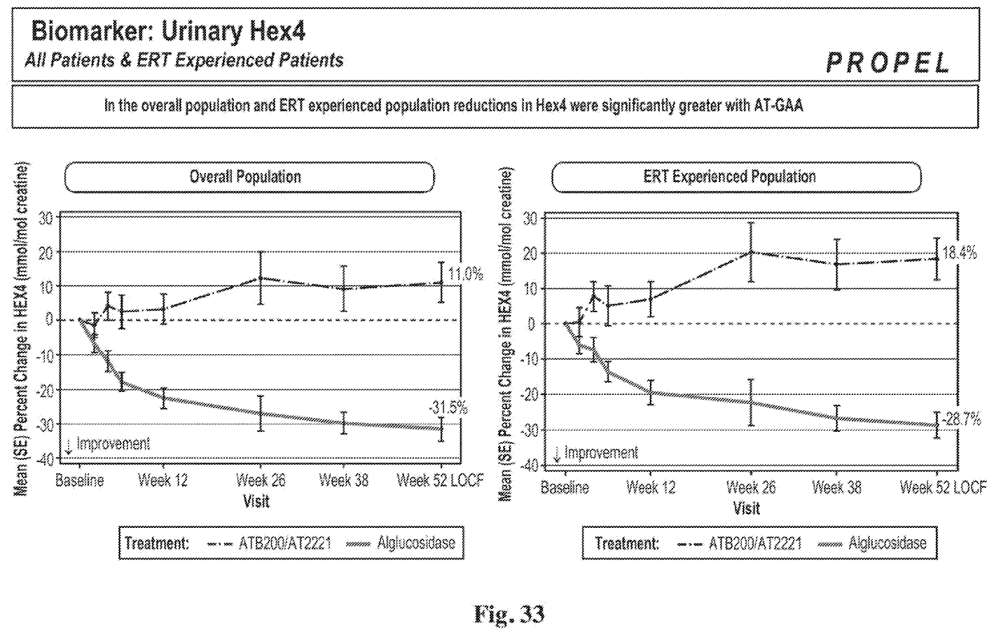

In the overall and ERT-experienced populations, ATB200/AT2221 treatment showed improvement in biomarkers of muscle damage (CK) and disease substrate (Hex4) over time (FIGS. 32 and 33). Furthermore, as shown in FIGS. 32 and 33, in the overall and ERT-experienced populations, reductions in CK and urinary Hex4 were significantly greater with ATB200/AT2221 treatment at week 52, compared to alglucosidase alfa/placebo.

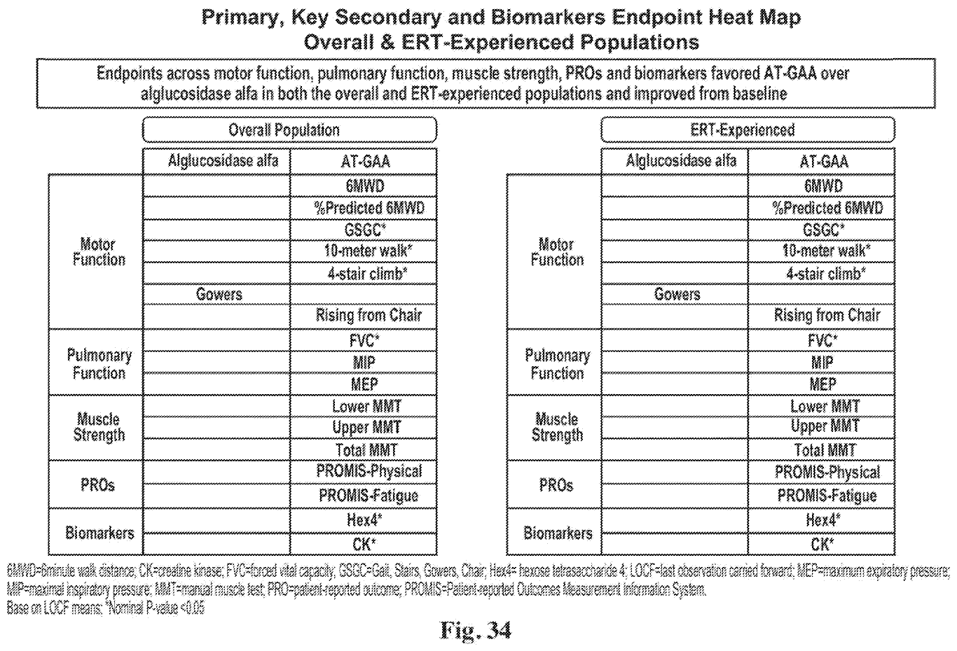

As summarized in FIG. 34, in the overall and ERT-experienced populations, endpoints across motor function, pulmonary function, muscle strength, patient-reported outcomes (PROs) and biomarkers consistently favored ATB200/AT2221 treatment over alglucosidase alfa/placebo. Furthermore, of the 17 efficacy and biomarker endpoints assessed, 16 favored ATB200/AT2221 treatment over alglucosidase alfa/placebo.

Safety Results from the ATB200-03 Trial

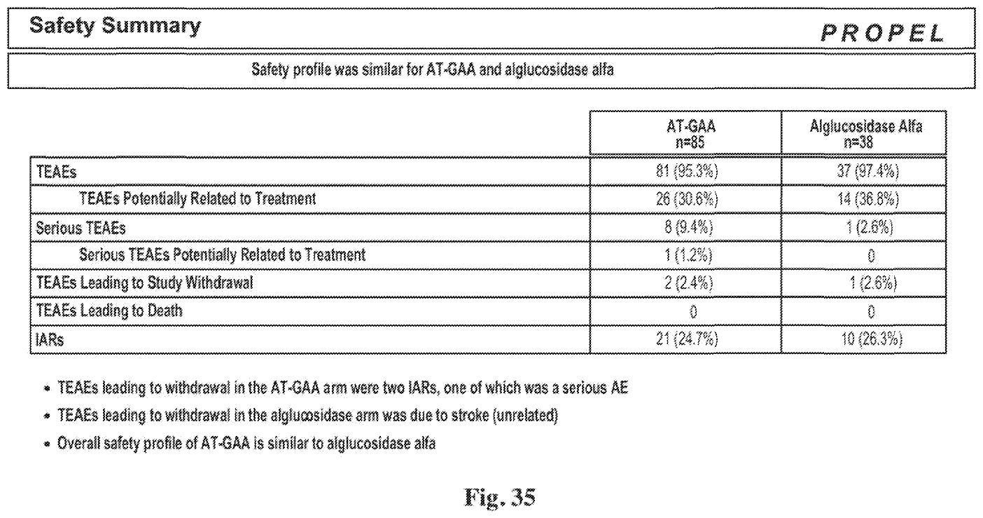

As shown in FIG. 35, the overall safety profile of ATB200/AT2221 treatment group was similar to that of the alglucosidase alfa/placebo group.

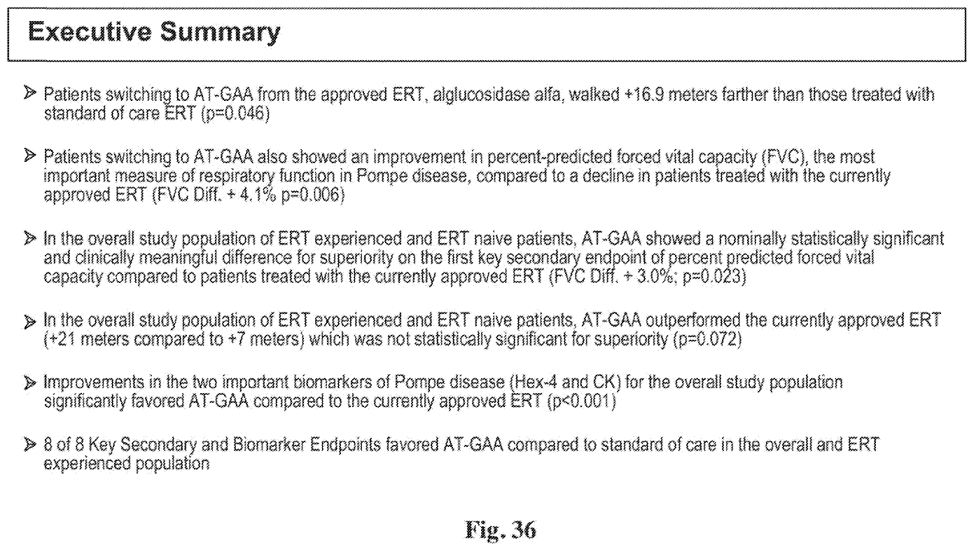

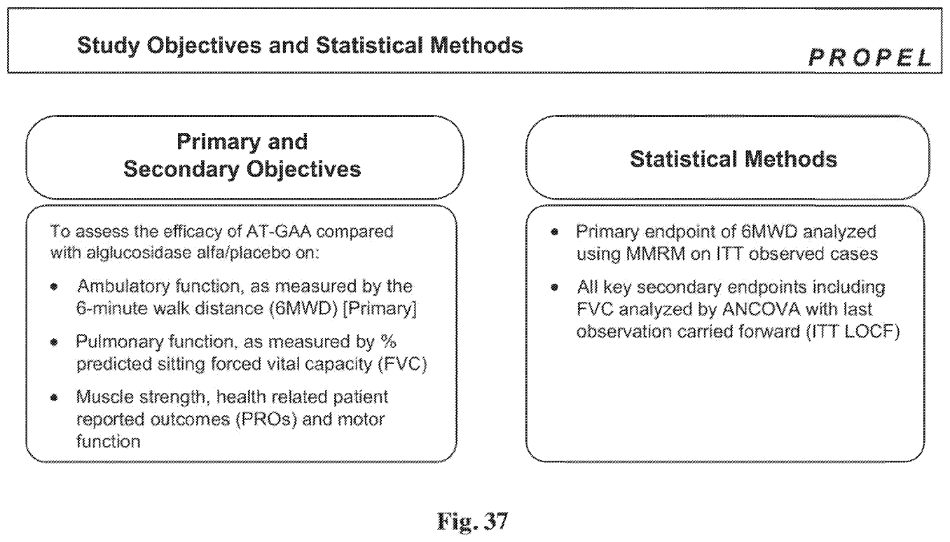

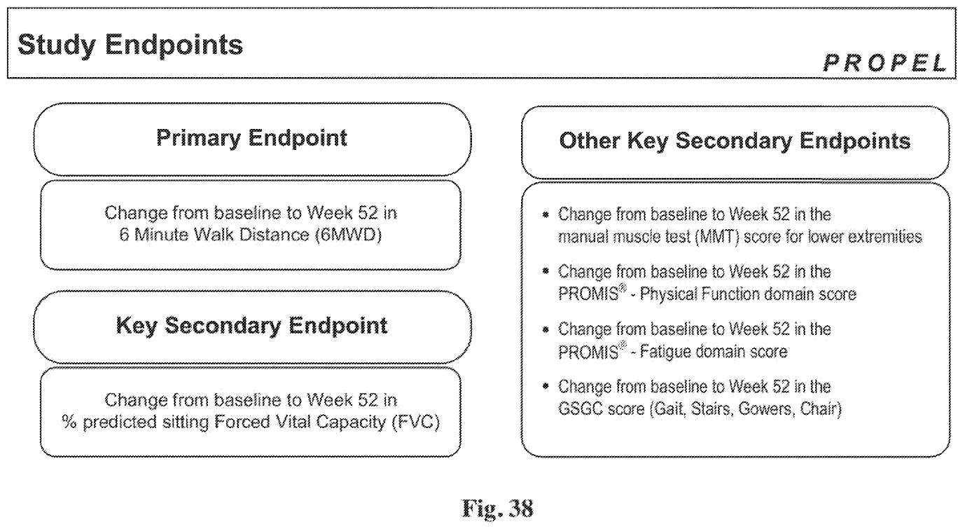

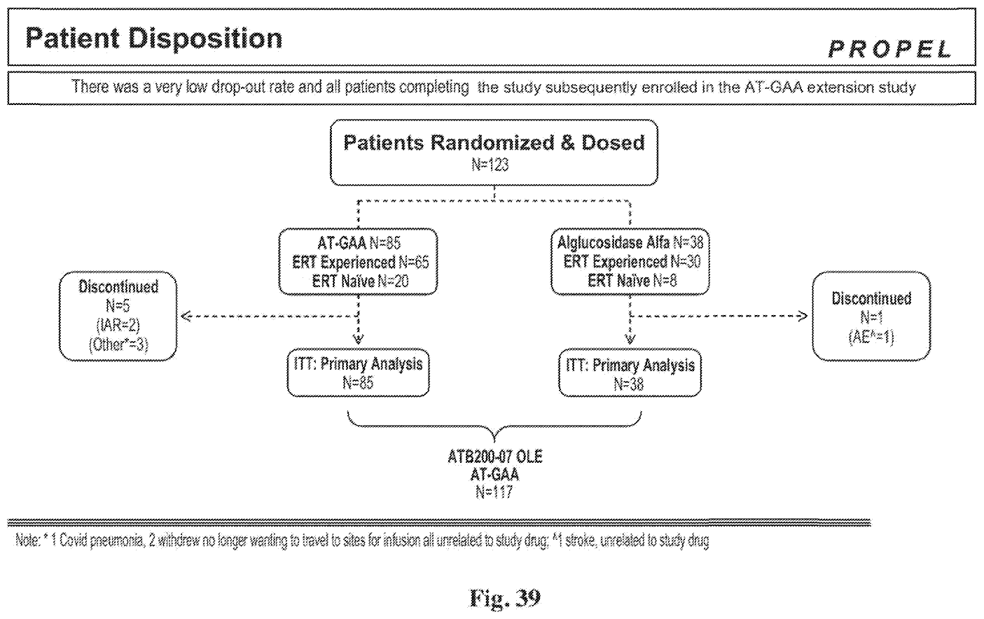

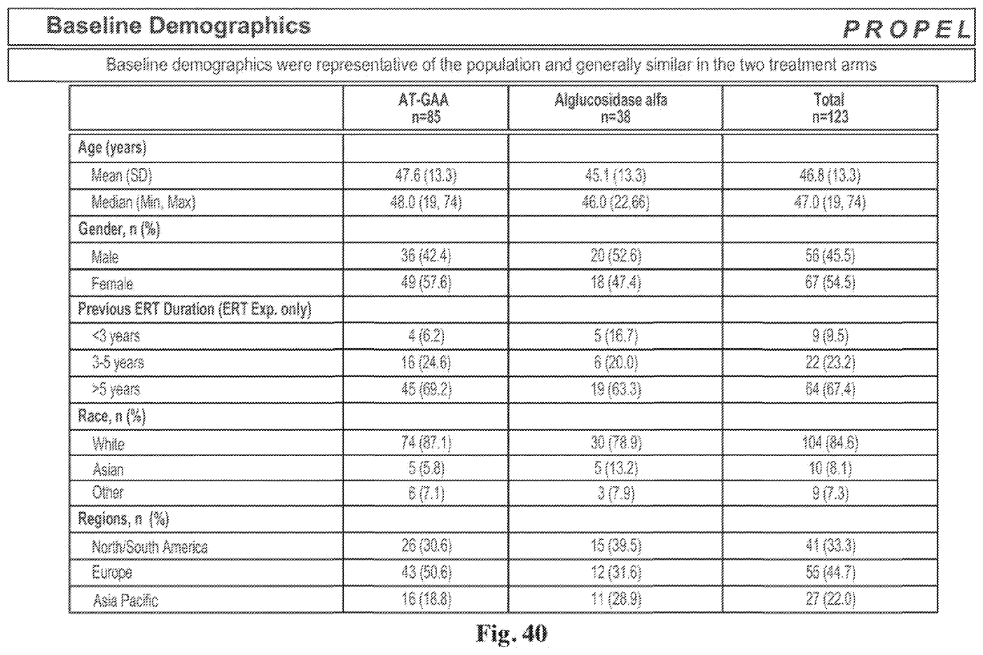

FIG. 36-FIG. 40 describe additional aspects of the ATB200-03 Trial.

### Example 10: Results of PROPEL Phase 3 Clinical Trial

AT-GAA showed clinically meaningful & significant improvements in both musculoskeletal and respiratory measures in late-onset Pompe disease compared to standard of care in pivotal phase 3 PROPEL study. PROPEL is also referred to as “ATB200-03”, see Example 9.

Patients switching to AT-GAA from the approved standard of care ERT (alglucosidase alfa) walked on average 17 meters farther (p=0.046).

Patients switching to AT-GAA also showed an improvement in percent-predicted forced vital capacity (FVC), the most important measure of respiratory function in Pompe disease, compared to a decline in patients treated with alglucosidase alfa (FVC Diff. 4.1%; p=0.006).

AT-GAA showed a nominally statistically significant and clinically meaningful difference for superiority on the first key secondary endpoint of FVC compared to patients treated with alglucosidase alfa (FVC Diff. 3.0%; p=0.023).

In the combined study population of ERT switch and ERT naïve patients, AT-GAA outperformed alglucosidase alfa by 14 meters (21 m compared to 7m) on the primary endpoint and was not statistically significant for superiority (p=0.072).

Improvements in the two important biomarkers of Pompe disease (Hex-4 and CK) for the combined study population significantly favored AT-GAA compared to alglucosidase alfa (p<0.001).

PROPEL was a 52-week, double-blind randomized global study designed to assess the efficacy, safety and tolerability of AT-GAA compared to the current standard of care, alglucosidase alfa, an enzyme replacement therapy (ERT). The study enrolled 123 adult Pompe patients who still had the ability to walk and to breathe without mechanical ventilation and was conducted at 62 clinical sites in 24 countries on 5 continents. It was the largest controlled clinical study ever conducted in a lysosomal disorder.

Patients enrolled in PROPEL were randomized 2:1 so that for every two patients randomized to be treated with AT-GAA, one was randomized to be treated with alglucosidase alfa. Of the Pompe patients enrolled in PROPEL, 77% were being treated with alglucosidase alfa (n=95) immediately prior to enrollment and 23% had never been treated with any ERT (n=28). 117 patients completed the PROPEL study and all 117 have voluntarily enrolled in the long-term extension study and are now being treated solely with AT-GAA for their Pompe disease.

### Pre-Specified Analyses of 6-Minute Walk Distance (6MWD) and Percent-Predicted Forced Vital Capacity (FVC) in the Combined ERT Switch and ERT Naïve Study Population:

The primary endpoint of the study was the mean change in 6-minute walk distance as compared with baseline measurements at 52 weeks across the combined ERT switch and ERT naïve patient populations. In this combined population patients taking AT-GAA (n=85) walked on average 21 meters farther at 52 weeks compared to 7 meters with those treated with alglucosidase alfa (n=37) (Table 10). This primary endpoint in the combined population was assessed for superiority and while numerically greater, statistical significance for superiority on this combined population was not achieved for the AT-GAA arm as compared to the alglucosidase alfa arm (p=0.072).

Per the hierarchy of the statistical analysis plan, the first key secondary endpoint of the study was the mean change in percent-predicted FVC at 52 weeks across the combined population. In this combined population patients taking AT-GAA demonstrated a nominally statistically significant and clinically meaningful difference for superiority over those treated with alglucosidase alfa. AT-GAA significantly slowed the rate of respiratory decline in patients after 52 weeks. Patients treated with AT-GAA showed a 0.9% absolute decline in percent-predicted FVC compared to a 4.0% absolute decline in the alglucosidase alfa arm (p=0.023) (Table 11). Percent-predicted FVC is the most important measure of respiratory muscle function in Pompe disease and was the basis of approval for alglucosidase alfa.

TABLE 10
6MWD (m) in the Overall ERT Switch and ERT Naïve Study Population

| | | CFBL at | | | |
|---|---|---|---|---|---|
| Treatment | Baseline | Week 52 | | Difference | P-Value |
| AT-GAA (n = 85) | 357.9 (111.8) | +20.8 | (4.6) | +13.6 (8.3) | p = 0.072 |
| Alglucosidase alfa | 351.0 (121.3) | +7.2 | (6.6) | | |
| (n = 37) | | | | | |

TABLE 11
FVC (% predicted) in the Overall ERT Switch
and ERT Naïve Study Population

| | | CFBL at | | |
|---|---|---|---|---|
| Treatment | Baseline | Week 52 | Difference | P-Value |
| AT-GAA (n = 85) | 70.7 (19.6) | −0.9 (0.7) | +3.0 (1.2) | p = 0.023 |
| Alglucosidase alfa | 69.7 (21.5) | −4.0 (0.8) | | |
| (n = 37) | | | | |

Pre-Specified Analyses of 6-Minute Walk Distance (6MWD) and Percent-Predicted Forced Vital Capacity (FVC) in the ERT Switch Study Population (n=95):

The PROPEL switch patients entered the study having been treated with alglucosidase alfa for a minimum of two years. More than two thirds (67%+) of those patients had been on ERT treatment for more than five years prior to entering the PROPEL study (mean of 7.4 years).

A pre-specified analysis of the patients switching from alglucosidase alfa on 6-minute walk distance showed that after 52 weeks from switching, AT-GAA treated patients (n=65) walked 16.9 meters farther than their baseline, compared to 0.0 meters for those patients who were randomized to remain on alglucosidase alfa (n=30) (p=0.046) (Table 12).

A pre-specified analysis of the patients switching from alglucosidase alfa on percent-predicted FVC showed that AT-GAA treated patients stabilized and slightly improved their respiratory function on this important measure while those patients remaining on alglucosidase alfa continued to significantly decline in respiratory muscle function. AT-GAA patients showed a 0.1% absolute increase in percent-predicted FVC while the alglucosidase alfa patients showed a 4.0% absolute decline over the course of the year (p=0.006) (Table 13).

TABLE 12
6MWD (m) in the ERT Switch Study Population

| | | CFBL at | | | |
|---|---|---|---|---|---|
| Treatment | Baseline | Week 52 | | Difference | P-Value |
| AT-GAA (n = 65) | 346.9 (110.2) | +16.9 | (5.0) | +16.9 (8.8) | p = 0.046 |
| Alglucosidase alfa | 334.6 (114.0) | 0.0 | (7.2) | | |
| (n = 30) | | | | | |

TABLE 13
FVC (% predicted) in the ERT Switch Study Population

| | | CFBL at | | |
|---|---|---|---|---|
| Treatment | Baseline | Week 52 | Difference | P-Value |
| AT-GAA (n = 65) | 67.9 (19.1) | +0.1 (0.7) | +4.1 (1.2) | p = 0.006 |
| Alglucosidase alfa | 67.5 (21.0) | −4.0 (0.9) | | |
| (n = 30) | | | | |

Pre-Specified Analyses of 6-Minute Walk Distance (6MWD) and Percent-Predicted Forced Vital Capacity (FVC) in the ERT Treatment Naive Population (n=28):

A pre-specified analysis of the patients previously never treated with any ERT on 6-minute walk distance showed that after 52 weeks AT-GAA treated patients (n=20) walked 33 meters farther than their baseline. The alglucosidase alfa treated patients (n=7) walked 38 meters further than their baseline. The difference between the two groups was not statistically significant (p=0.60) (Table 14).

A pre-specified analysis of the patients never previously treated with any ERT showed similar declines in percent-predicted forced vital capacity (FVC) at 52 weeks of −4.1% for AT-GAA treated patients and −3.6% for alglucosidase alpha treated patients (Table 15). The difference between the two groups was not statistically significant (p=0.57).

TABLE 14
6MWD (m) in the ERT Treatment Naïve Population

| | | CFBL at | | |
|---|---|---|---|---|
| Treatment | Baseline | Week 52 | Difference | P-Value |
| AT-GAA (n = 20) | 393.6 (112.4) | +33.4 (10.9) | −4.9 (19.7) | p = 0.60 |
| Alglucosidase alfa | 420.9 (135.7) | +38.3 (11.1) | | |
| (n = 7) | | | | |

TABLE 15
FVC (% predicted) in the ERT Treatment Naïve Population

| | | CFBL at | | |
|---|---|---|---|---|
| Treatment | Baseline | Week 52 | Difference | P-Value |
| AT-GAA (n = 20) | 80.2 (18.7) | −4.1 (1.5) | −0.5 (2.7) | p = 0.57 |
| Alglucosidase alfa | 79.1 (22.6) | −3.6 (1.8) | | |
| (n = 7) | | | | |

Note:
One patient in the alglucosidase alfa arm was excluded from the study analysis due to use of an investigational anabolic like steroid that impacted his baseline performance.

### Pre-Specified Analyses of Other Key Secondary and Biomarker Endpoints Across the Overall ERT Switch and ERT Naïve Study Population:

### Musculoskeletal & Other Key Secondary Endpoints:

GSGC (Gait, Stairs, Gower's Chair): GSGC is an important and commonly used endpoint in Pompe Disease capturing strength, coordination and mobility. AT-GAA treated patients demonstrated statistically significant improvements on the scores in this important assessment, compared to a worsening for alglucosidase alfa treated patients in the overall population (p<0.05).

Lower MMT (Manual Muscle Testing), PROMIS Physical Function: On both of these validated measures of muscle strength and patient reported outcomes, AT-GAA treated patients improved numerically more than alglucosidase alfa treated patients, though the results were not statistically significant.

PROMIS Fatigue: Fatigue as measured by this scale slightly favored AT-GAA treated patients over alglucosidase alfa treated patients.

### Biomarkers of Treatment Effects on Disease:

Urine Hex-4: For the combined study population of both ERT switch and ERT naïve patients, those patients receiving AT-GAA showed substantial improvements on this biomarker, with a mean reduction of Hex-4 of −31.5% after 52 weeks compared to an increase of +11.0% (i.e., worsening) in Hex-4 in the alglucosidase alfa treated patients (p=<0.001). Urine Hex-4 is a common biomarker in Pompe disease and is used as an indirect measure of the degree of skeletal glycogen clearance in Pompe patients receiving ERT. Glycogen is the substrate that accumulates in the lysosomes of muscles of Pompe patients.

CK (Creatine Kinase): After 52 weeks, AT-GAA treated patients showed substantial improvements on this biomarker as well with a mean −22.4% reduction in CK compared to an increase (i.e., worsening) of +15.6% in the alglucosidase alfa treated patients. (p<0.001). CK is an enzyme that leaks out of damaged muscle cells and is elevated in Pompe patients.

AT-GAA demonstrated a similar safety profile to alglucosidase alfa. Two patients receiving AT-GAA (2.4%) discontinued treatment due to an adverse event compared to one (2.6%) for alglucosidase alfa unrelated to treatment. Injection associated reactions (IARs) were reported in 25% of AT-GAA participants and 26% of alglucosidase alfa patients.

### Post Hoc Subgroup Analyses:

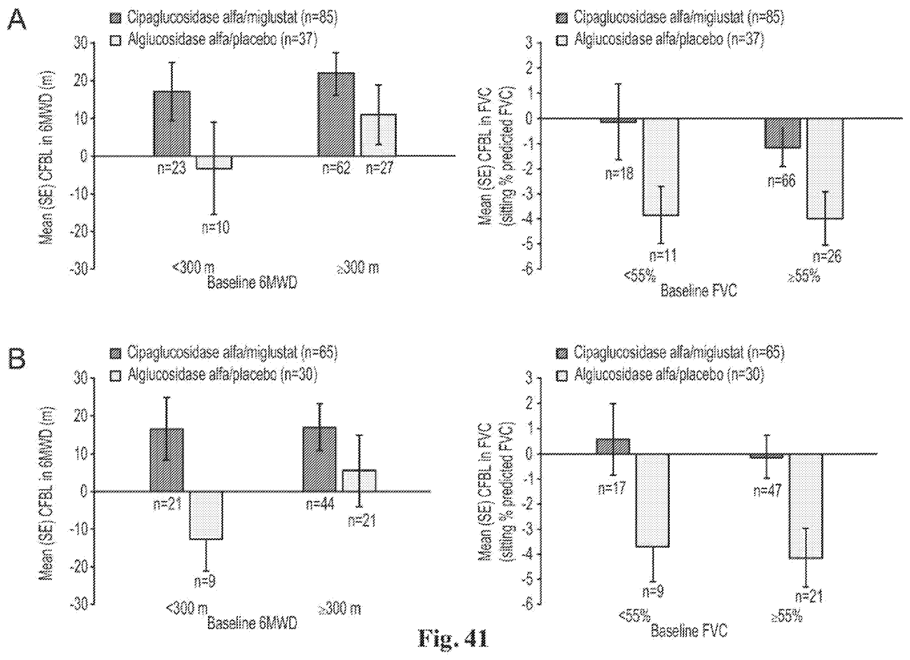

Baseline 6MWD and FVC categories: ERT-naïve population (n=27): three patients had a baseline 6MWD of <300 m and three had a baseline FVC of <55%; analyses of CFBL were not performed in these subgroups owing to the small patient numbers. Baseline 6MWD≥300 m: both the cipaglucosidase alfa/miglustat (AT-GAA) (n=18) and alglucosidase alfa/placebo (n=6) groups had similar improvements over time (mean [SE] CFBL to week 52: +34.4 [12.1] m and +30.8 [9.6] m, respectively). Baseline FVC≥55%: both the cipaglucosidase alfa/miglustat (n=19) and alglucosidase alfa/placebo (n=5) groups declined over time (mean [SE] CFBL to week 52: −3.7 [1.5]% and −3.3 [2.6]%, respectively). Outcomes consistently favored cipaglucosidase alfa/miglustat in the overall and ERT-experienced populations in patients with baseline 6MWD of <300 m and >300 m, and FVC of <55% and ≥55%, as shown in FIG. 41A and FIG. 41B.

In the overall study population including ERT-naïve and ERT-experienced patients, cipaglucosidase alfa/miglustat showed positive trends or clinically meaningful improvements on motor and respiratory functions compared with approved ERT, regardless of baseline 6MWD and % FVC assessments, and across both prespecified and post hoc subgroup analyses.

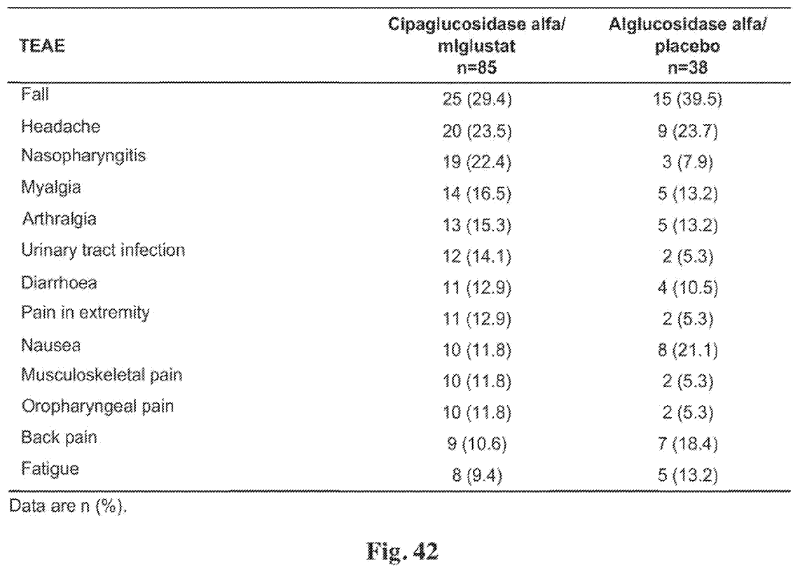

Cipaglucosidase alfa/miglustat demonstrated a similar safety profile to that of alglucosidase alfa/placebo (FIG. 42).

### About AT-GAA

AT-GAA is an investigational two-component therapy that consists of cipaglucosidase alfa (ATB200), a unique recombinant human acid alpha-glucosidase (rhGAA) enzyme with optimized carbohydrate structures, particularly bis-phosphorylated mannose-6 phosphate (bis-M6P) glycans, to enhance uptake into cells, administered in conjunction with miglustat (AT2221), a stabilizer of cipaglucosidase alfa. In preclinical studies, AT-GAA was associated with increased levels of the mature lysosomal form of GAA and reduced glycogen levels in muscle, alleviation of the autophagic defect and improvements in muscle strength.

### About Pompe Disease

Pompe disease is an inherited lysosomal disorder caused by deficiency of the enzyme acid alpha-glucosidase (GAA). Reduced or absent levels of GAA levels lead to accumulation of glycogen in cells, which is believed to result in the clinical manifestations of Pompe disease. The disease can be debilitating and is characterized by severe muscle weakness that worsens over time. Pompe disease ranges from a rapidly fatal infantile form with significant impacts to heart function to a more slowly progressive, late-onset form primarily affecting skeletal muscle. It is estimated that Pompe disease affects approximately 5,000 to 10,000 people worldwide.

### NUMBERED EMBODIMENTS

Notwithstanding the appended claims, the disclosure sets forth the following numbered embodiments:

1. A method of treating Pompe disease in a subject in need thereof, comprising administering to the subject a population of recombinant human acid α-glucosidase (rhGAA) molecules, concurrently or sequentially with a pharmacological chaperone;
wherein the rhGAA molecules comprise seven potential N-glycosylation sites;
wherein 40%-60% of the N-glycans on the rhGAA molecules are complex type N-glycans;
wherein the rhGAA molecules comprise at least 0.5 mol bis-mannose-6-phosphate (bis-M6P) per mol of rhGAA at the first potential N-glycosylation site as determined using liquid chromatography tandem mass spectrometry (LC-MS/MS); and
wherein the method improves one or more disease outcomes the subject compared to (1) baseline, or (2) a control treatment comprising administering alglucosidase alfa and a placebo for the pharmacological chaperone.

2. The method of embodiment 1, wherein the method improves the subject's motor function, as measured by a 6-minute walk test.

3. The method of embodiment 2, wherein the change from baseline in 6-minute walk distance (6MWD) is at least 20 meters.

4. The method of embodiment 3, wherein the change from baseline in 6MWD is at least 20 meters after 52 weeks of treatment.

5. The method of embodiment 2, wherein the subject's 6MWD is increased by at least 10 compared to the control treatment.

6. The method of embodiment 5, wherein, compared to the control treatment, the subject's 6MWD is improved by at least 13 meters after 52 weeks of treatment.

7. The method of any one of embodiments 2-6, wherein the subject has a baseline 6MWD less than 300 meters.

8. The method of any one of embodiments 2-6, wherein the subject has a baseline 6MWD greater than or equal to 300 meters.

9. The method of embodiment 1, wherein the method improves the subject's pulmonary function, as measured by a forced vital capacity (FVC) test.

10. The method of embodiment 9, wherein, after treatment, the subject's percent-predicted FVC is either increased compared to baseline, or decreased by less than 3% compared to baseline.

11. The method of embodiment 10, wherein, after treatment, the subject's percent-predicted FVC is decreased by less than 1% compared to baseline.

12. The method of embodiment 10 or embodiment 11, wherein the subject's percent-predicted FVC is decreased by less than 1% compared to baseline after 52 weeks of treatment.

13. The method of embodiment 9, wherein, compared to the control treatment, the subject's percent-predicted FVC is significantly improved or stabilized after treatment.

14. The method of embodiment 13, wherein, compared to the control treatment, the subject's percent-predicted FVC is improved by at least 3% after treatment.

15. The method of embodiment 12 or embodiment 13, wherein, compared to the control treatment, the subject's percent-predicted FVC is improved by at least 3% after 52 weeks of treatment.

16. The method of any one of embodiments 9-15, wherein the subject has a baseline FVC less than 55%.

17. The method of any one of embodiments 9-15, wherein the subject has a baseline FVC greater than or equal to 55%.

18. The method of embodiment 1, wherein the method improves the subject's motor function, as measured by a gait, stair, gower, chair (GSGC) test.

19. The method of embodiment 18, wherein, compared to baseline, the subject's GSGC score is improved as indicated by a decrease of at least 0.5 point after treatment.

20. The method of embodiment 19, wherein, compared to baseline, the subject's GSGC score is improved as indicated by a decrease of at least 0.5 point after 52 weeks of treatment.

21. The method of embodiment 18, wherein, compared to the control treatment, the subject's GSGC score is significantly improved after treatment.

22. The method of embodiment 21, wherein, compared to the control treatment, the subject's GSGC score is improved as indicated by a decrease of at least 1 point after treatment.

23. The method of embodiment 21 or embodiment 22, wherein, compared to the control treatment, the subject's GSGC score is improved as indicated by a decrease of at least 1 point after 52 weeks of treatment.

24. The method of embodiment 1, wherein the method reduces the levels of at least one marker of muscle damage and/or at least one marker of glycogen accumulation.

25. The method of embodiment 24, wherein the at least one marker of muscle damage comprises creatine kinase (CK), and/or the at least one marker of glycogen accumulation comprises urine hexose tetrasaccharide (Hex4).

26. The method of embodiment 25, wherein, compared to baseline, the subject's CK level is reduced by at least 20% after treatment, and/or the subject's urinary Hex4 level is reduced by at least 30% after treatment.

27. The method of embodiment 26, wherein, compared to baseline, the subject's CK level is reduced by at least 20% after 52 weeks of treatment, and/or the subject's urinary Hex4 level is reduced by at least 30% after 52 weeks of treatment.

28. The method of embodiment 25, wherein, compared to the control treatment, the subject's CK and/or urinary Hex4 level is significantly reduced after treatment.

29. The method of embodiment 28, wherein, compared to the control treatment, the subject's CK level is reduced by at least 30% after treatment, and/or the subject's urinary Hex4 level is reduced by at least 40% after treatment.

30. The method of embodiment 28 or embodiment 29, wherein, compared to the control treatment, the subject's CK level is reduced by at least 30% after 52 weeks of treatment, and/or the subject's urinary Hex4 level is reduced by at least 40% after 52 weeks of treatment.

31. The method of any one of embodiments 1-30, wherein the subject is an ERT-experienced patient.

32. The method of any one of embodiments 1-30, wherein the subject is an ERT-naive patient.

33. A method of treating Pompe disease in a subject in need thereof, comprising administering to the subject a population of recombinant human acid α-glucosidase (rhGAA) molecules, concurrently or sequentially with a pharmacological chaperone;
wherein the rhGAA molecules comprise seven potential N-glycosylation sites;
wherein 40%-60% of the N-glycans on the rhGAA molecules are complex type N-glycans;
wherein the rhGAA molecules comprise at least 0.5 mol bis-mannose-6-phosphate (bis-M6P) per mol of rhGAA at the first potential N-glycosylation site as determined using liquid chromatography tandem mass spectrometry (LC-MS/MS);
wherein the method improves one or more disease symptoms in the subject compared to (1) baseline, or (2) a control treatment comprising administering alglucosidase alfa and a placebo for the pharmacological chaperone, and
wherein the subject is an ERT-experienced patient.

34. The method of embodiment 33, wherein the method improves the subject's motor function, as measured by a 6-minute walk test.

35. The method of embodiment 34, wherein, compared to baseline, the subject's 6-minute walk distance (6MWD) is increased by at least 15 meters or at least 5% after treatment.

36. The method of embodiment 35, wherein, compared to baseline, the subject's 6-minute walk distance (6MWD) is increased by at least 15 meters or at least 4% after 52 weeks of treatment.

37. The method of embodiment 33, wherein, compared to the control treatment, the subject's 6MWD is significantly improved after treatment.

38. The method of embodiment 37, wherein, compared to the control treatment, the subject's 6MWD is improved by at least 15 meters after treatment.

39. The method of embodiment 37 or embodiment 38, wherein, compared to the control treatment, the subject's 6MWD is improved by at least 15 meters after 52 weeks of treatment.

40. The method of any one of embodiments 34-39, wherein the subject has a baseline 6MWD less than 300 meters.

41. The method of any one of embodiments 34-39, wherein the subject has a baseline 6MWD greater than or equal to 300 meters.

42. The method of embodiment 33, wherein the method improves the subject's pulmonary function, as measured by a forced vital capacity (FVC) test.

43. The method of embodiment 42, wherein, after treatment, the subject's percent-predicted FVC is increased by at least 0.1% compared to baseline.

44. The method of embodiment 43, wherein the subject's percent-predicted FVC is increased by at least 0.1% compared to baseline after 52 weeks of treatment.

45. The method of embodiment 42, wherein, compared to the control treatment, the subject's percent-predicted FVC is significantly improved after treatment.

46. The method of embodiment 45, wherein, compared to the control treatment, the subject's percent-predicted FVC is improved by at least 4% after treatment.

47. The method of embodiment 45 or embodiment 46, wherein, compared to the control treatment, the subject's percent-predicted FVC is improved by at least 4% after 52 weeks of treatment.

48. The method of any one of embodiments 42-47, wherein the subject has a baseline FVC less than 55%.

49. The method of any one of embodiments 42-47, wherein the subject has a baseline FVC greater than or equal to 55%.

50. The method of embodiment 33, wherein the method improves the subject's motor function, as measured by a gait, stair, gower, chair (GSGC) test.

51. The method of embodiment 50, wherein, compared to baseline, the subject's GSGC score is improved as indicated by a decrease of at least 0.5 point after treatment.

52. The method of embodiment 51, wherein, compared to baseline, the subject's GSGC score is improved as indicated by a decrease of at least 0.5 point after 52 weeks of treatment.

53. The method of embodiment 50, wherein, compared to the control treatment, the subject's GSGC score is significantly improved after treatment.

54. The method of embodiment 53, wherein, compared to the control treatment, the subject's GSGC score is improved as indicated by a decrease of at least 1 point after treatment.

55. The method of embodiment 53 or embodiment 54, wherein, compared to the control treatment, the subject's GSGC score is improved as indicated by a decrease of at least 1 point after 52 weeks of treatment.

56. The method of embodiment 33, wherein the method reduces the levels of at least one marker of muscle damage and/or at least one marker of glycogen accumulation.

57. The method of embodiment 56, wherein the at least one marker of muscle damage comprises creatine kinase (CK), and/or the at least one marker of glycogen accumulation comprises urine hexose tetrasaccharide (Hex4).

58. The method of embodiment 57, wherein, compared to baseline, the subject's CK level is reduced by at least 15% after treatment, and/or the subject's urinary Hex4 level is reduced by at least 25% after treatment.

59. The method of embodiment 58, wherein, compared to baseline, the subject's CK level is reduced by at least 15% after treatment, and/or the subject's urinary Hex4 level is reduced by at least 25% after 52 weeks of treatment.

60. The method of embodiment 57, wherein, compared to the control treatment, the subject's CK and/or urinary Hex4 level is significantly reduced after treatment.

61. The method of embodiment 60, wherein, compared to the control treatment, the subject's CK level is reduced by at least 30% after treatment, and/or the subject's urinary Hex4 level is reduced by at least 40% after treatment.

62. The method of embodiment 60 or embodiment 61, wherein, compared to the control treatment, the subject's CK level is reduced by at least 30% after treatment, and/or the subject's urinary Hex4 level is reduced by at least 40% after 52 weeks of treatment.

63. The method of any one of embodiments 1-62, wherein the population of rhGAA molecules is administered at a dose of 5 mg/kg to 20 mg/kg, optionally 20 mg/kg.

64. The method of any one of embodiments 1-63, wherein the population of rhGAA molecules is administered bi-weekly.

65. The method of any one of embodiments 1-64, wherein the population of rhGAA molecules is administered intravenously.

66. The method of any one of embodiments 1-65, wherein the pharmacological chaperone is miglustat or a pharmaceutically acceptable salt thereof, wherein further optionally the miglustat or pharmaceutically acceptable salt thereof is administered orally.

67. The method of embodiment 66, wherein the miglustat or pharmaceutically acceptable salt thereof is administered at a dose of 195 mg or 260 mg.

68. The method of embodiment 66 or embodiment 67, wherein the miglustat or pharmaceutically acceptable salt thereof is administered prior to administration of the population of rhGAA molecules, optionally one hour prior to administration of the population of rhGAA molecules.

69. The method of embodiment 68, wherein the subject fasts for at least two hours before and at least two hours after the administration of miglustat or a pharmaceutically acceptable salt thereof.

70. The method of any one of embodiments 1-69, wherein the rhGAA molecules comprise an amino acid sequence at least 95% identical to SEQ ID NO: 4 or SEQ ID NO: 6.

71. The method of any one of embodiments 1-70, wherein the rhGAA molecules comprise the amino acid sequence of SEQ ID NO: 4 or SEQ ID NO: 6.

72. The method of any one of embodiments 1-71, wherein at least 30% of the rhGAA molecules comprise one or more N-glycan units bearing one mannose-6-phosphate residue (mono-M6P) or bis-M6P, as determined using LC-MS/MS.

73. The method of any one of embodiments 1-72, wherein the rhGAA molecules comprise on average from 0.5 mol to 7.0 mol of mono-M6P or bis-M6P per mol of rhGAA, as determined using LC-MS/MS.

74. The method of any one of embodiments 1-73, wherein the rhGAA molecules comprise on average from 2.0 to 8.0 mol of sialic acid per mol of rhGAA, as determined using LC-MS/MS.

75. The method of any one of embodiments 1-73, wherein the rhGAA molecules comprise on average at least 2.5 mol M6P per mol of rhGAA and at least 4 mol sialic acid per mol of rhGAA, as determined using LC-MS/MS.

76. The method of any one of embodiments 1-75, wherein, per mol of rhGAA, the rhGAA molecules comprise on average:
(a) 0.4 to 0.6 mol mono-M6P at the second potential N-glycosylation site;
(b) 0.4 to 0.6 mol bis-M6P at the fourth potential N-glycosylation site; or
(c) 0.3 to 0.4 mol mono-M6P at the fourth potential N-glycosylation site;
wherein (a)-(c) are determined using LC-MS/MS.

77. The method of embodiment 76, wherein, per mol of rhGAA, the rhGAA molecules further comprise 4 mol to 7.3 mol sialic acid; and wherein, per mol of rhGAA, the rhGAA molecules comprise on average:
(a) 0.9 to 1.2 mol sialic acid at the third potential N-glycosylation site;
(b) 0.8 to 0.9 mol sialic acid at the fifth potential N-glycosylation site; or
(c) 1.5 to 4.2 mol sialic acid at the sixth potential N-glycosylation site;
wherein (a)-(c) are determined using LC-MS/MS.

78. The method of any one of embodiments 1-77, wherein the population of rhGAA molecules is formulated in a pharmaceutical composition.

79. The method of embodiment 78, wherein the pharmaceutical composition further comprises at least one buffer selected from the group consisting of a citrate, a phosphate, and a combination thereof, and at least one excipient selected from the group consisting of mannitol, polysorbate 80, and a combination thereof; wherein the pharmaceutical composition has a pH of 5.0 to 7.0.

80. The method of embodiment 79, wherein the pharmaceutical composition has a pH of 5.0 to 6.0.

81. The method of embodiment 78 or embodiment 79, wherein the pharmaceutical composition further comprises water, an acidifying agent, an alkalizing agent, or a combination thereof.

82. The method of embodiment 81, wherein, in the pharmaceutical composition, the population of rhGAA molecules is present at a concentration of 5-50 mg/mL, the at least one buffer is a sodium citrate buffer present at a concentration of 10-100 mM, the at least one excipient is mannitol present at a concentration of 10-50 mg/mL and polysorbate 80 present at a concentration of 0.1-1 mg/mL, and the pharmaceutical composition further comprises water and optionally comprises an acidifying agent and/or alkalizing agent; wherein the pharmaceutical composition has a pH of 6.0.

83. The method of embodiment 82, wherein, in the pharmaceutical composition, the population of rhGAA molecules is present at a concentration of 15 mg/mL, the sodium citrate buffer is present at a concentration of 25 mM, the mannitol is present at a concentration of 20 mg/mL, and the polysorbate 80 is present at a concentration of 0.5 mg/mL.

84. The method of any one of embodiments 1-83, wherein the rhGAA is produced from Chinese hamster ovary cells.

1. A method of treating Pompe disease in a subject in need thereof, comprising administering to the subject a population of recombinant human acid α-glucosidase (rhGAA) molecules, concurrently or sequentially with a pharmacological chaperone;
wherein the rhGAA molecules comprise seven potential N-glycosylation sites;
wherein 40%-60% of the N-glycans on the rhGAA molecules are complex type N-glycans;
wherein the rhGAA molecules comprise at least 0.5 mol bis-mannose-6-phosphate (bis-M6P) per mol of rhGAA at the first potential N-glycosylation site as determined using liquid chromatography tandem mass spectrometry (LC-MS/MS); and
wherein the method improves one or more disease outcomes the subject compared to (1) baseline, or (2) a control treatment comprising administering alglucosidase alfa and a placebo for the pharmacological chaperone.

2. The method of claim 1, wherein the method improves the subject's motor function, as measured by a 6-minute walk test.

3. The method of claim 2, wherein the change from baseline in 6-minute walk distance (6MWD) is at least 20 meters.

4. (canceled)

5. The method of claim 2, wherein the subject's 6MWD is increased by at least 10 compared to the control treatment.

6. (canceled)

7. The method of claim 1, wherein the subject has a baseline 6MWD less than 300 meters.

8. The method of claim 1, wherein the subject has a baseline 6MWD greater than or equal to 300 meters.

9. The method of claim 1, wherein the method improves the subject's pulmonary function, as measured by a forced vital capacity (FVC) test.

10. The method of claim 9, wherein, after treatment, the subject's percent-predicted FVC is either increased compared to baseline, or decreased by less than 3% compared to baseline.

11. The method of claim 10, wherein, after treatment, the subject's percent-predicted FVC is decreased by less than 1% compared to baseline.

12. (canceled)

13. (canceled)

14. The method of claim 9, wherein, compared to the control treatment, the subject's percent-predicted FVC is improved by at least 3% after treatment.

15. (canceled)

16. The method of claim 1, wherein the subject has a baseline FVC less than 55%.

17. The method of claim 1, wherein the subject has a baseline FVC greater than or equal to 55%.

18. The method of claim 1, wherein the method improves the subject's motor function, as measured by a gait, stair, gower, chair (GSGC) test.

19. The method of claim 18, wherein, compared to baseline, the subject's GSGC score is improved as indicated by a decrease of at least 0.5 point after treatment.

20. (canceled)

21. (canceled)

22. The method of claim 2418, wherein, compared to the control treatment, the subject's GSGC score is improved as indicated by a decrease of at least 1 point after treatment.

23. (canceled)

24. (canceled)

25. The method of claim 241, wherein the method reduces the levels of at least one marker of muscle damage comprising creatine kinase (CK), and/or the method reduces the levels of at least one marker of glycogen accumulation comprising urine hexose tetrasaccharide (Hex4).

26. The method of claim 25, wherein, compared to baseline, the subject's CK level is reduced by at least 20% after treatment, and/or the subject's urinary Hex4 level is reduced by at least 30% after treatment.

27. (canceled)

28. (canceled)

29. The method of claim 25, wherein, compared to the control treatment, the subject's CK level is reduced by at least 30% after treatment, and/or the subject's urinary Hex4 level is reduced by at least 40% after treatment.

30. (canceled)

31. The method of claim 1, wherein the subject is an ERT-experienced patient.

32. The method of claim 1, wherein the subject is an ERT-naive patient.

33-84. (canceled)


SEQUENCE LISTING (7 sequences)

SEQ ID NO: 1 - protein, 952 aa, Artificial Sequence
  recombinant human acid alpha-glucosidase
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - DNA, 3624 bp, Artificial Sequence
  recombinant human acid alpha-glucosidase
cagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 3 - protein, 952 aa, Artificial Sequence; identical to SEQ ID NO: 1
  recombinant human acid alpha-glucosidase

SEQ ID NO: 4 - protein, 952 aa, Artificial Sequence
  recombinant human acid alpha-glucosidase
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 5 - protein, 896 aa, Artificial Sequence
  recombinant human acid alpha-glucosidase
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 6 - protein, 896 aa, Artificial Sequence
  recombinant human acid alpha-glucosidase
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 7 - protein, 6 aa, Unknown
  conserved hexapeptide
WIDMNE